(19) United States
(12) Patent Application Publication
(10) Pub. No.: US 2013/0004579 A1
(43) Pub. Date: Jan. 3, 2013

(54) Use of Plant Lectins to Target Leukocytes

(76) Inventors: Edward C. Lavelle, Dublin (IE); Edel McNeela, Dublin (IE); Darren Thomas Ruane, Kildare (IE); Christopher Davitt, Dublin (IE); Karen Misstear, Wicklow (IE)

(21) Appl. No.: 13/470,477

(22) Filed: May 14, 2012

Related U.S. Application Data
(60) Provisional application No. 61/485,653, filed on May 13, 2011.

(51) Int. Cl. A61K 39/385; A61P 37/04; A61K 9/14

(52) U.S. Cl. 424/491; 424/193.1

(57) ABSTRACT

The present invention provides compositions and methods for targeting an antigen to leukocytes, delivering an antigen to leukocytes, increasing antigen uptake by leukocytes, and/or enhancing an immune response. In some embodiments, compositions and methods of the present invention comprise a conjugate comprising an antigen and a plant lectin or a mimetic thereof.

### RELATED APPLICATIONS

This application claims priority under 35 U.S.C. §119(e) to U.S. Provisional Patent Application No. 61/485,653, filed on May 13, 2011, the disclosure of which is incorporated herein by reference in its entirety.

### FIELD OF THE INVENTION

The present invention relates to compositions for targeting and/or delivering an antigen to leukocytes and methods of using the same.

### BACKGROUND

The vertebrate immune system is a complex and diverse collection of cells and organs that work together to eliminate exogenous and endogenous threats from the host. In order to deal with an ever changing spectrum of potential threats, the immune system has evolved into two distinguishable sub-systems, differentiated by their respective levels of detection and effector specificity. The innate immune system contains a limited number of receptors, while the adaptive immune system contains a highly specific, extremely variable repertoire of receptors. Although the receptors of the innate system are fewer and less specific than those of the adaptive system, they are constitutively expressed and can respond rapidly when activated. The innate immune system acts as a constitutively active sentinel, rapidly containing and identifying threats and quickly activating and instructing the adaptive system to mount the most effective response against a particular pathogen and to allow for clearance, healing, and the generation of future immunity.

Dendritic cells (DC) are central to the induction of antigen-specific immune responses and the priming of T cell-mediated immunity. As members of the innate immune system, dendritic cells specialize in antigen (Ag) uptake, processing and presentation and act as a bridge between the innate and the adaptive immune systems.

Although dendritic cells are widely distributed throughout the body, they are not stationary sentinels. Indeed, they are highly mobile. Upon encountering and uptake of an antigen, they migrate from the site of the encounter to lymphoid organs and present the antigen to naive T cells, thereby inducing or suppressing an immune response.

Despite the development of various methods of delivering antigens to dendritic cells, there is a continuing need for novel and efficacious compositions and methods for targeting and/or delivering antigens to dendritic cells and other antigen-presenting cells.

### SUMMARY OF THE INVENTION

Compositions and methods for targeting and/or delivering antigens to leukocytes are provided. The compositions and methods of the present invention may result in an increase in the number of leukocytes taking up the antigen and/or an increase in the amount of antigen taken up per leukocyte.

Compositions of the present invention may comprise, consist essentially of or consist of an antigen and a plant lectin or a mimetic thereof. In some embodiments, the antigen and the plant lectin or mimetic thereof form a conjugate. In some embodiments, the composition comprises a conjugate comprising an antigen, a plant lectin or a mimetic thereof and a particle, wherein the antigen and the plant lectin or mimetic thereof are each attached to the particle.

Compositions of the present invention may be used to target an antigen to leukocytes, to deliver an antigen to leukocytes, to increase the uptake of an antigen by leukocytes, to stimulate a T cell response (e.g., a Type 1 helper T cell (T_{H}1) response and/or a Type 17 helper T cell (T_{H}17) response) in a subject and/or to enhance an immune response to an antigen in a subject. Accordingly, methods of the present invention may comprise, consist essentially of or consist of administering to a subject a composition of the present invention and/or contacting a leukocyte with a medium comprising a composition of the present invention. In some embodiments, methods of the present invention result in an enhanced cellular immune response in the absence of an enhanced humoral immune response.

These and other objects and aspects of the present invention will be appreciated by those of skill in the art from a reading of the figures and the detailed description set forth below.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F show that targeting with Ulex europaeus agglutinin 1 (UEA-1) increases polystyrene particle uptake by dendritic cells in vitro.

FIGS. 2A-2F show that targeting with UEA-1 increases polystyrene particle uptake by dendritic cells in vitro.

FIG. 3 shows that the conjugation of UEA-1 to polystyrene particles increases uptake by dendritic cells after a 1 hour incubation.

FIG. 4 shows that the conjugation of UEA-1 to polystyrene particles increases uptake by dendritic cells after a 2 hour incubation.

FIGS. 5A-5B show that the conjugation of UEA-1, soybean agglutinin (SBA), Phaseolus vulgaris erthyroagglutinin (PHA-E), Phaseolus vulgaris leukoagglutinin (PHA-L) or Datura stramonium lectin (DSL) to polystyrene particles increases uptake of the particles by dendritic cells after a 30 minute incubation.

FIGS. 6A-6B show that the conjugation of UEA-1, SBA, PHA-E, PHA-L or DSL to polystyrene particles increases uptake of the particles by macrophages after a 10 minute incubation.

FIGS. 7A-7F show that UEA-1, SBA, PHA-E, PHA-L and DSL increase polystyrene particle uptake (at various concentrations of particles) by dendritic cells after a 30 minute incubation.

FIGS. 8A-8H show that UEA-1, SBA, PHA-E, PHA-L and DSL increase polystyrene particle uptake (at various concentrations of particles) by macrophages after a 10 or 30 minute incubation.

FIGS. 9A-9C show that UEA-1 targeting enhances polystyrene particle uptake by various splenocyte populations in vitro.

FIG. 10 shows that UEA-1 targeting increases the number of polystyrene particles taken up per cell by phagocytic splenocyte populations in vitro.

FIGS. 11A-11D show that the conjugation of UEA-1, SBA, PHA-E, PHA-L or DSL to polystyrene particles increases uptake of the particles by multiple spleen cell populations.

FIG. 12 shows that the conjugation of UEA-1, SBA, PHA-E, PHA-L or DSL to polystyrene particles increases uptake of the particles by multiple spleen cell populations.

FIGS. 13A-13B show that targeting polystyrene particles with UEA-1 increases IL-1α and IL-1β cytokine production by dendritic cells in vitro. *** represents p<0.001.

FIGS. 14A-14B show that adsorbing UEA-1 to polystyrene particles enhances IL-1α and IL-1β production by dendritic cells in vitro. * represents p<0.05. *** represents p<0.001.

FIGS. 15A-15B show that UEA-1 does not significantly enhance alum-mediated IL-1 production by dendritic cells in vitro.

FIGS. 16A-16B show that targeting with a UEA-1 mimetic enhances the polystyrene particle-mediated enhancement of IL-1α and IL-β production by dendritic cells in vitro. *** represents p<0.001.

FIGS. 17A-17B show that UEA-1 induces stronger polystyrene particle-mediated enhancement of IL-1α and IL-1β production by dendritic cells than a UEA-1 mimetic in vitro. *** represents p<0.001.

FIGS. 18A-18B show that TLR-2 agonist-primed IL-1α and IL-β production by dendritic cells is increased by UEA1-targeting in vitro. *** represents p<0.001.

FIGS. 19A-19H show that TLR-4 agonist-primed IL-1α production by dendritic cells is increased by in vitro targeting of particles with PHA-L, PHA-E, Dolichos biflorus agglutinin (DBA), concanavalin A (Con A), wheat germ agglutinin (WGA), peanut agglutinin (PNA), UEA-1, Pisum sativum lectin (PSA), Lycopersicon esculentum lectin (LEL), Vicia villoa lectin (VVL), Jacalin (Jac), Griffonia simplicifolia lectin II (GSL II), Griffonia simplicifolia lectin I (GSL I), SBA or DSL.

FIGS. 20A-20F show that TLR-4 agonist-primed IL-β production by dendritic cells is increased by in vitro targeting of particles with PHA-L, PHA-E, VVL, SBA, PSA, GSL I, UEA-1, DBA, Con A, WGA, PNA or GSL II.

FIGS. 21A-21D show that in vitro targeting of particles with PHA-L enhances IL-1α production but not IL-1β production by dendritic cells in the absence of NLRP3.

FIGS. 22A-22D show that in vitro targeting of particles with PHA-E enhances IL-1α production but not IL-β production by dendritic cells in the absence of NLRP3.

FIGS. 23A-23D show that in vitro targeting of particles with UEA-1 enhances IL-1α production but not IL-1β production by dendritic cells in the absence of NLRP3.

FIGS. 24A-24D show that in vitro targeting of particles with SBA enhances IL-1α production but not IL-1β production by dendritic cells in the absence of NLRP3.

FIG. 25 shows that targeting polystyrene particles with UEA-1 and a UEA-1 mimetic increases active IL-β secretion by LPS-primed dendritic cells in vitro.

FIGS. 26A-26C show that attachment of UEA-1 or a UEA-1 mimetic to polystyrene particles with antigen does not significantly increase antigen-specific IgG antibody responses in mice in vivo following i.p. administration. * represents p<0.05.

FIGS. 27A-27D show that targeting of antigen-loaded polystyrene particles with UEA-1 enhances antigen-specific cytokine responses in murine spleens following i.p. administration.

FIGS. 28A-28D show that targeting of antigen-loaded polystyrene particles with UEA-1 or a UEA-1 mimetic enhances antigen-specific cytokine responses in murine peritoneal cells following i.p. administration.

FIG. 29 shows that targeting polystyrene particles with UEA-1 mimetic increases IL-1α and IL-1β secretion by LPS-primed dendritic cells in vitro in an NLRP3-dependent manner. * represents p<0.05. ** represents p<0.01. *** represents p<0.001.

FIGS. 30A-30B show that intranasally immunizing mice with UEA-1 targeted particles coated with OVA induces IL-17 and IFNγ production in antigen-specific CD3⁺CD8⁺ T cells isolated from the mediastinal lymph nodes of mice in an NLRP3-dependent manner. Data are presented as mean (±SEM), tested individually in triplicate. * represents p<0.05.

FIG. 31 shows that UEA-1 mimetic increases chitosan-driven IL-β secretion by LPS-primed dendritic cells in vitro in an NLRP3-independent manner. Data are presented as mean (±SEM) cytokine concentrations for each sample tested individually in triplicate.

FIG. 32 shows that intranasally immunizing mice with UEA-1 targeted particles coated with ClfA increases antigen-specific IL-17 and IFNγ secretion by splenocytes. Data are presented as mean (±SEM) cytokine concentrations for each sample tested individually in triplicate.

FIG. 33 shows that intranasally immunizing mice with UEA-1 targeted particles coated with ClfA induces IL-17 and IFNγ production in antigen-specific CD3⁺CD4⁺ T cells and CD3⁺CD8⁺ T cells isolated from the mediastinal lymph nodes of mice. Data from five mice per treatment group were pooled and presented as mean (±SEM).

FIG. 34 shows that lectin-targeted particles enhance the production of antigen-specific antibodies following i.p. immunization.

FIGS. 35A-35B show that targeting streptavidin-coated polystyrene particles with UEA-1 or UEA-1 mimetic increases both antigen-specific and nonspecific IFNγ production in splenocytes (FIG. 35A) and peritoneal exudate cells (FIG. 35B) following i.p. immunization. Data are presented as mean (±SEM) cytokine concentrations from five mice per experimental treatment group tested individually in triplicate. ** represents p<0.05. ***represents p<0.001.

FIGS. 36A-36B show that targeting streptavidin-coated polystyrene particles with UEA-1 or UEA-1 mimetic increases both antigen-specific (FIG. 36A) and nonspecific (FIG. 36B) IL-17 production in peritoneal exudate cells following i.p, immunization. Data are presented as mean (±SEM) cytokine concentrations from five mice per experimental treatment group tested individually in triplicate.

FIGS. 37A-37B show that targeting streptavidin-coated polystyrene particles with PHA-L or SBA increases both antigen-specific (FIG. 37A) and nonspecific (FIG. 37B) IL-4 production in peritoneal exudate cells following i.p. immunization. Data are presented as mean (±SEM) cytokine concentrations from five mice per experimental treatment group tested individually in triplicate. ***represents p<0.001.

FIGS. 38A-38B show that targeting streptavidin-coated polystyrene particles with PHA-L or SBA does not alter antigen-specific IL-10 production (FIG. 38A), but does increase nonspecific IL-10 production in peritoneal exudate cells (FIG. 38B) following i.p. immunization. Data are presented as mean (±SEM) cytokine concentrations from five mice per experimental treatment group tested individually in triplicate. **represents p<0.01.

### DETAILED DESCRIPTION

The foregoing and other aspects of the present invention will now be described in more detail with respect to the description and methodologies provided herein. This description is not intended to be a detailed catalogue of all the ways in which the present invention may be implemented, or of all the features that may be added to the present invention. For example, features illustrated with respect to one embodiment may be incorporated into other embodiments, and features illustrated with respect to a particular embodiment may be deleted from that embodiment. In addition, numerous variations and additions to the various embodiments suggested herein, which do not depart from the instant invention, will be apparent to those skilled in the art in light of the instant disclosure. Hence, the following specification is intended to illustrate some particular embodiments of the invention, and not to exhaustively specify all permutations, combinations and variations thereof.

All patents, patent publications, non-patent publications and sequences referenced herein are incorporated by reference in their entireties.

### DEFINITIONS

Although the following terms are believed to be well understood by one of skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

As used herein, the terms “a” or “an” or “the” may refer to one or more than one. For example, “a” marker can mean one marker or a plurality of markers.

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, the term “adjuvant” refers to a material that enhances the immune response to a given antigen without giving rise to its own specific antigenic activity. Thus, a material that does not enhance the immune response to a given antigen would not be considered an adjuvant. Likewise, a material that elicits its own specific antigenic activity would not be considered an adjuvant, even if it enhances the immune response to a given antigen.

As used herein, the term “and/or” refers to and encompasses any and all possible combinations of one or more of the associated listed items, as well as the lack of combinations when interpreted in the alternative (“or”).

As used herein, the term “consists essentially of” (and grammatical variants thereof), as applied to the compositions and methods of the present invention, means that the compositions/methods may contain additional components so long as the additional components do not materially alter the composition/method. The term “materially alter,” as applied to a composition/method, refers to an increase or decrease in the effectiveness of the composition/method of at least about 20% or more. For example, a component added to a composition of the present invention would “materially alter” the composition if it increases or decreases the composition's ability to induce an immune response by 50%.

As used herein, the term “effective amount” refers to an amount that imparts a desired effect. In some embodiments, the desired effect comprises a therapeutic effect and/or a prophylactic effect.

As used herein, the term “enhanced cellular immune response” refers to an increase in at least one aspect of a cellular immune response. In some embodiments, a plant lectin is deemed to produce an enhanced cellular immune response if at least one aspect of a cellular immune response is increased by at least about 5%, 10%, 20%, 30% or more (as compared to the cellular immune response in the absence of the plant lectin). For example, an enhanced cellular immune response to a given antigen may comprise a 20% increase in antigen-specific cytokine responses. In some embodiments, an enhanced cellular immune response comprises an increase in the production and/or secretion of IL-1α, IL-1β, IFN-γ, IL-5, IL-10 and/or IL-17. In some embodiments, an enhanced cellular immune response comprises an increase in cytotoxicity (e.g., antibody-dependent cell-mediated cytotoxicity, lymphocyte-mediated cytotoxicity and/or complement-dependent cytotoxicity), phagocytosis and/or chemotaxis.

As used herein, the term “enhanced humoral immune response” refers to an increase in at least one aspect of a humoral immune response. In some embodiments, a plant lectin is deemed to produce an enhanced humoral immune response if at least one aspect of a humoral immune response is increased by at least about 5%, 10%, 20%, 30% or more (as compared to the humoral immune response in the absence of the plant lectin). For example, an enhanced humoral immune response to a given antigen may comprise a 20% increase in the production of antibodies that are specific to that antigen.

As used herein, the term “enhanced immune response” refers to an increase in at least one aspect of an immune response, including, but not limited to, a cellular immune response or a humoral immune response. In some embodiments, a plant lectin is deemed to produce an enhanced immune response if at least one aspect of an immune response is increased by at least about 5%, 10%, 20%, 30% or more (as compared to the immune response in the absence of the plant lectin). For example, a plant lectin may be deemed to produce an enhanced immune response if conjugation of the plant lectin to an antigen produces a significant increase in antigen-specific cytokine responses and/or a significant increase in the production of antibodies that are specific to that antigen. The enhanced immune response may comprise an enhanced protective immune response and/or an enhanced therapeutic immune response.

As used herein, the term “emulsion” refers to a suspension or dispersion of one liquid within a second immiscible liquid. In some embodiments, the emulsion is an oil-in-water emulsion or a water-in-oil emulsion. In some embodiments, “emulsion” may refer to a material that is a solid or semi-solid at room temperature and is a liquid at body temperature (about 37° C.).

As used herein, the term “liposome” refers to an aqueous or aqueous-buffered compartment enclosed by a lipid bilayer. In general, liposomes can be prepared by a thin film hydration technique followed by a few freeze-thaw cycles. Liposomal suspensions can also be prepared according to other methods known to those skilled in the art.

As used herein, the term “micelle” refers to an aqueous or aqueous-buffered compartment enclosed by an aggregate of surfactant molecules (e.g., fatty acids, salts of fatty acids or phospholipids). Micelle suspensions may be prepared according to any suitable method known to those of skill in the art.

As used herein, the term “microparticle” refers to a particle that is about 1 μm to about 1 mm in diameter.

As used herein, the term “mimetic” refers to a compound whose structure is such that it acts as a functional equivalent of at least one function of a second compound, performing essentially the same function(s) as the second compound in essentially the same way(s) with essentially the same result(s). For example, a plant lectin mimetic (e.g., a UEA-1 mimetic) may be a compound that performs at least one of the same biological functions as a plant lectin (e.g., UEA-1) in essentially the same way with essentially the same results (e.g., the mimetic may bind the same cell surface receptor(s) as the plant lectin, thereby inducing essentially the same cellular response(s) as would occur if the plant lectin itself was bound to the receptor(s)). In some instances, there may be no appreciable difference in the response(s) elicited by the mimetic and the plant lectin itself (e.g., no statistical difference between the amounts of IL-1α produced by dendritic cells). In other instances, there may be an appreciable difference in the response(s) elicited by the mimetic and the lectin (e.g., a statistically significant difference in IL-1α production of about 0.5%, 1%, 5%, 10%, 20%, 30%, 40% or even 50% or more). In some instances, the response elicited by the mimetic may be at least about 20% that of the response elicited by the plant lectin itself (e.g., the amount of IL-1α produced by dendritic cells in response to mimetic-targeted particles may be at least about 20%, 30%, 40%, 50%, 60%, 70%, 80%, 90%, 95%, 100% or more as compared to the amount of IL-1α produced by dendritic cells in response to particles targeted with the plant lectin itself).

As used herein, the term “nanoparticle” refers to a particle that is about 1 nm to about 1 μm in diameter.

As used herein, “pharmaceutically acceptable” means that the material is suitable for administration to a subject and will allow desired treatment to be carried out without giving rise to unduly deleterious side effects. The severity of the disease and the necessity of the treatment are generally taken into account when determining whether any particular side effect is unduly deleterious.

As used herein, the terms “prevent,” “preventing,” and “prevention” (and grammatical variants thereof) refer to avoidance, prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods of the present invention. In some embodiments, prevention is complete, resulting in the total absence of the disease, disorder and/or clinical symptom(s). In some embodiments, prevention is partial, resulting in reduced severity and/or delayed onset of the disease, disorder and/or clinical symptom(s).

As used herein, the term “prevention effective amount” (and grammatical variants thereof) refers an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

As used herein, “subject” (and grammatical variants thereof) refers to mammals, avians, reptiles, amphibians, or fish. Mammalian subjects may include, but are not limited to, humans, non-human primates (e.g., monkeys, chimpanzees, baboons, etc.), dogs, cats, mice, hamsters, rats, horses, cows, pigs, rabbits, sheep and goats. Avian subjects may include, but are not limited to, chickens, turkeys, ducks, geese, quail and pheasant, and birds kept as pets (e.g., parakeets, parrots, macaws, cockatoos, and the like). In particular embodiments, the subject is from an endangered species. In particular embodiments, the subject is a laboratory animal. Human subjects may include neonates, infants, juveniles, adults, and geriatric subjects.

As used herein, the terms “therapeutically effective amount” and “therapeutically acceptable amount” (and grammatical variants thereof) refer to an amount that will elicit a therapeutically useful response in a subject. The therapeutically useful response may provide some alleviation, mitigation, or decrease in at least one clinical symptom in the subject. The terms also include an amount that will prevent or delay at least one clinical symptom in the subject and/or reduce and/or delay the severity of the onset of a clinical symptom in a subject relative to what would occur in the absence of the methods of the invention. Those skilled in the art will appreciate that the therapeutically useful response need not be complete or curative or prevent permanently, as long as some benefit is provided to the subject.

As used herein, the terms “treatment,” “treat,” and “treating” (and grammatical variants thereof) refer to reversing, alleviating, delaying the onset of, inhibiting the progress of or preventing a disease or disorder. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved—for example, to prevent or delay their recurrence.

As used herein, the term “treatment effective amount” (and grammatical variants thereof) refers to an amount that is sufficient to provide some improvement or benefit to the subject. Alternatively stated, a “treatment effective amount” is an amount that will provide some alleviation, mitigation, decrease, or stabilization in at least one clinical symptom in the subject. Those skilled in the art will appreciate that the therapeutic effects need not be complete or curative, as long as some benefit is provided to the subject.

### Compositions

The present invention provides compositions for targeting and/or delivering an antigen to leukocytes, wherein the compositions comprise an antigen and a plant lectin or a mimetic thereof.

Any suitable antigen may be used, including, but not limited to, an antigen of an intracellular pathogen, an antigen of an extracellular pathogen, a cancer or tumor antigen, a hormone or an allergen.

Examples of suitable antigens include, but are not limited to, orthomyxovirus antigens (e.g., an influenza virus antigen, such as the influenza virus hemagglutinin (HA) surface protein, influenza neuraminidase or the influenza virus nucleoprotein, or an equine influenza virus antigen), lentivirus antigens (e.g., an equine infectious anaemia virus antigen, a Simian Immunodeficiency Virus (SIV) antigen, or a Human Immunodeficiency Virus (HIV) antigen, such as the HIV or SIV envelope GP160 protein, the HIV or SIV matrix/capsid proteins, and the HIV or SIV gag, pol and env gene products), arenavirus antigens (e.g., Lassa fever virus antigen, such as the Lassa fever virus nucleocapsid protein and the Lassa fever envelope glycoprotein), poxvirus antigens (e.g., a vaccinia virus antigen, such as the vaccinia L1 or L8 gene products), flavivirus antigens (e.g., a yellow fever virus antigen or a Japanese encephalitis virus antigen), Filovirus antigens (e.g., an Ebola virus antigen, or a Marburg virus antigen, such as NP and GP gene products), bunyavirus antigens (e.g., RVFV, CCHF, and/or SFS virus antigens), coronavirus antigens (e.g., an infectious human coronavirus antigen, such as the human coronavirus envelope glycoprotein, or a porcine transmissible gastroenteritis virus antigen, or an avian infectious bronchitis virus antigen), polio antigens, herpes antigens (e.g., CMV, EBV, HSV antigens), human papilloma virus (HPV) antigens, rabies antigens, tick-borne encephalitis antigens, meningococcal antigens, tetanus antigens, pneumococcal antigens, tuberculosis antigens, cholera antigens, staphylococcal antigens, shigella antigens, vesicular stomatitis antigens, mumps antigens, measles antigens, rubella antigens, diphtheria toxin or other diphtheria antigens, pertussis antigens, hepatitis (e.g., hepatitis A, hepatitis B, hepatitis C, etc.) antigens, retinal antigens and/or any other antigen now known in the art or later identified as an antigen.

Exemplary cancer and tumor cell antigens are described by S. A. Rosenberg (IMMUNITY 10:281 (1991)). Other illustrative cancer and tumor antigens include, but are not limited to, alphafetoprotein, carcinoembryonic antigen, prostate-specific antigen, MUC-1, epithelial tumor antigen, CA 15-3, squamous cell carcinoma antigen, bladder tumor associated antigen, BRCA1 gene product, BRCA2 gene product, gp100, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., PROC. NATL. ACAD. SCI. USA 91:3515 (1994); Kawakami et al., J. Exp. Med. 180:347 (1994); Kawakami et al., Cancer Res. 54:3124 (1994)), MART-1, gp100 MAGE-1, MAGE-2, MAGE-3, CEA, TRP-1, TRP-2, P-15, tyrosinase (Brichard et al., J. EXP. MED. 178:489 (1993)); HER-2/neu gene product (U.S. Pat. No. 4,968,603), CA-125, CA 27.29, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, ANN. REV. BIOCHEM. 62:623 (1993)); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukaemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, ANN. REV. MED. 47:481-91 (1996)).

Exemplary allergens include, but are not limited to, pollen (e.g., grass, weed, tree or plant pollen), epithelial cells (e.g., cat, dog, rat and pig epithelia), dust, dust mite excretion, bee or wasp venom, basidiospores, Aspergillus, Coprinus comatus and wheat chaff.

The antigen may be targeted and/or delivered to any suitable leukocyte(s), including, but not limited to, lymphoblasts, granulocytes (including neutrophils, basophils and/or eosinophils), antigen-presenting cells (including dendritic cells, macrophages and/or B cells), monocytes, and microglia. In some embodiments, leukocytes comprise leukocytes other than T cells. In some embodiments, the leukocytes are phagocytic leukocytes. In some embodiments, the leukocytes are selected from the group consisting of dendritic cells, monocytes and granulocytes. In some embodiments, the leukocytes are dendritic cells.

Any suitable plant lectin or mimetic may be used, including, but not limited to, Aleuria aurantia lectin (AAL), Amaranthus caudatus lectin (ACL), Bauhinia purpurea lectin (BPL), Caragana arborescens lectin (CAL), Con A, DBA, DSL, Erythrina cristagalli lectin (ECL), Euonymus europaeus lectin (EEL), Galanthus nivalis lectin (GNL), GSL I, GSL II, Hippeastrum hybrid lectin (HHL), Jac, LEL, Lens culinaris agglutinin (LCA), Lotus tetragonolobus lectin (LTL), Maackia amurensis lectin I (MAL I), Maackia amurensis lectin II (MAL II), Maclura pomifera lectin (MPL), mistletoe lectin I (ML-I), mistletoe lectin II (ML-II), mistletoe lectin III (ML-III), Narcissus pseudonarcissus lectin (NPL), Phaseolus lunatus lectin (PLL), Phaseolus vulgaris agglutinin (PHA), PHA-E, PHA-L, PNA, PSA, Psophocarpus tetragonolobus lectin I (PTL I), Psophocarpus tetragonolobus lectin II (PTL II), Ricinus communis agglutinin I (RCA I), Ricinus communis agglutinin II (RCA II), SBA, Sambucus nigra lectin (SNA), Solanum tuberosum lectin (STL), Sophora japonica agglutinin (SJA), UEA-1, Vicia faba lectin (VFL), VVL, Vigna radiata lectin I (MBL-I), Vigna radiata lectin II (MBL-II), WGA, Wisteria floribunda lectin (WFL) and mimetics thereof. See generally U.S. Pat. No. 6,863,896; Lavelle et al. SCANDANAVIAN J. IMMUNOL. 52:422 (2000); Lavelle et al. IMMUNOL. 102:77 (2001); Lavelle et al. IMMUNOL. 107:268 (2002); Misumi et al. J. IMMUNOL. 182:6061 (2009); Shibuya et al. J. BIOL. CHEM. 262:1596 (1987); Stein et al. ANTI-CANCER DRUGS 8:S57 (1997). In some embodiments, the plant lectin (or mimetic) is Con A, DBA, DSL, GSL I, GSL II, Jac, LEL, PHA-E, PHA-L, PNA, PSA, SBA, UEA-1, VVL or WGA (or a mimetic of one or more of the aforementioned lectins).

Any suitable method may be used to create and/or identify a suitable plant lectin mimetic, including, but not limited to, the methods described by Mazik (CHEMBIOCHEM 9:1015-1017 (2008)) and Lambkin et al, (PHARM. RES. 20:1258-1266 (2003)). See also U.S. Pat. No. 7,166,296. The plant lectin or mimetic thereof may or may not act as adjuvant. In some embodiments, the plant lectin or mimetic thereof targets leukocytes, but does not act as an adjuvant.

The antigen and the plant lectin or mimetic thereof may be combined in any suitable manner known in the art, including, but not limited to, incorporation of the antigen and the plant lectin or mimetic thereof into a solution/suspension and/or formation of a conjugate comprising the antigen and the plant lectin or mimetic thereof. Any suitable method known in the art may be used to conjugate the antigen and the plant lectin or mimetic thereof. For example, the antigen and the plant lectin or mimetic thereof may be directly coupled (by a shared covalent or non-covalent bond, for example). Alternatively, the antigen and the plant lectin or mimetic thereof may be indirectly coupled (i.e., one or more molecules is interposed between the antigen and the plant lectin or mimetic thereof). In some embodiments, the antigen and the plant lectin or mimetic thereof are conjugated using one or more ester, ether and/or amide linkages. In some embodiments, conjugation of the antigen and the plant lectin or mimetic thereof may be facilitated by the addition of one or more amine groups to the antigen and/or the plant lectin or mimetic thereof. One skilled in the art will understand how to select a suitable conjugation method, taking into account numerous factors, including, but not limited to, the identity of the antigen and the identity of the plant lectin or mimetic thereof.

The composition may comprise any suitable pharmaceutical carrier, including, but not limited to, phosphate buffered saline and isotonic saline solution. Other examples of pharmaceutically acceptable carriers may be found, for example, in ANSEL'S PHARMACEUTICAL DOSAGE FORMS AND DRUG DELIVERY SYSTEMS (9th Ed., Lippincott Williams and Wilkins (2010)), PHARMACEUTICAL SCIENCES (18th Ed., Mack Publishing Co. (1990) or REMINGTON: THE SCIENCE AND PRACTICE OF PHARMACY (21st Ed., Lippincott Williams & Wilkins (2005)).

The composition may comprise any suitable diluent or excipient, including, but not limited to, those set forth in ANSEL'S PHARMACEUTICAL DOSAGE FORMS AND DRUG DELIVERY SYSTEMS (9th Ed., Lippincott Williams and Wilkins (2010)), HANDBOOK OF PHARMACEUTICAL EXCIPIENTS (6th Ed., American Pharmaceutical Association (2009)) and REMINGTON: THE SCIENCE AND PRACTICE OF PHARMACY (21st Ed., Lippincott Williams & Wilkins (2005)).

The composition may be formulated so as to be suitable for administration via any known method, including, but not limited to, oral, intravenous (i.v.), subcutaneous, intramuscular, intrathecal, intraperitoneal (i.p.), intrarectal, intravaginal, intranasal, intragastric, intratracheal, sublingual, transcutaneous and intrapulmonary. In some embodiments, the composition is formulated for intraperitoneal administration (e.g., intraperitoneal injection). In some embodiments, the composition is formulated for intranasal administration.

The composition may comprise any suitable adjuvant, including, but not limited to, alum (e.g., aluminium phosphate or aluminium hydroxide), squalene, an emulsion, a liposome, a micelle, and a particle (e.g., a metallic oxide particle, a biocompatible polymer particle, a solid lipid particle, etc.). In some embodiments, the adjuvant is a microparticle or a nanoparticle. In some embodiments, the adjuvant is a polystyrene (PS) particle, a chitosan particle, a polysaccharide particle (e.g., a starch, sugar or glycosoaminoglycan particle) a poly(glycolic acid) (PGA) particle, a poly(lactic acid) (PLA) particle or a poly(lactic-co-glycolic acid) (PLGA) particle.

Liposomes

The antigen and/or the plant lectin or mimetic thereof may be associated with a liposome. In some embodiments, the antigen is contained within the liposome (e.g., within the lipid bilayer or within the aqueous lumen of the liposome). In some embodiments, the antigen and/or the plant lectin or mimetic thereof is embedded in or attached to the surface of the liposome. In some embodiments, both the antigen and the plant lectin or mimetic thereof are embedded in or attached to the surface of the liposome. In some embodiments, the antigen and the plant lectin or mimetic thereof are in a solution/suspension that comprises one or more liposomes.

The antigen and/or the plant lectin or mimetic thereof may be associated with the liposome using any suitable means known in the art. For example, they may be encapsulated by the liposome as it forms, embedded in the surface of the liposome (e.g., a hydrophobic portion of the antigen may be embedded in the lipid bilayer whilst a hydrophilic portion of the antigen extends outwardly from the surface of the liposome) or attached to the surface of the liposome. They may be attached to the surface of the liposome directly (e.g., they may be adsorbed to the surface of the liposome or they may form a covalent or non-covalent bond with the surface of the liposome) or indirectly (i.e., one or more linker molecules may be interposed between the surface of the liposome and the antigen and/or the plant lectin or mimetic thereof).

In some embodiments, the antigen is encapsulated within the aqueous lumen of a liposome as it forms and the plant lectin or mimetic thereof is embedded in or attached (either directly or indirectly) to the surface to the liposome. In some embodiments, both the antigen and the plant lectin or mimetic thereof are embedded in or attached (either directly or indirectly) to the surface of the liposome. For example, an antigen may be adsorbed to the surface of the liposome whilst UEA-1 or a mimetic thereof is attached to the liposome via a linker molecule embedded in the lipid bilayer.

In some embodiments, an antigen and/or a plant lectin or a mimetic thereof is conjugated to an individual monomeric lipid and combined into a self-assembling spheroid particle. In some embodiments, both the antigen and the plant lectin or mimetic thereof are conjugated to monomeric lipids and combined into a self-assembling spheroid particle. For example, an antigen and UEA-1 or a mimetic thereof may each be conjugated to a distinct monomeric lipid and then mixed with a sufficient number of additional monomeric lipids to form a liposome comprising the antigen and UEA-1 or the mimetic thereof.

Micelles

The antigen and/or the plant lectin or mimetic thereof may be associated with a micelle. In some embodiments, the antigen is contained within the micelle (e.g., within the aqueous lumen of the micelle). In some embodiments, the antigen and/or the plant lectin or mimetic thereof is embedded in or attached to the surface of the micelle. In some embodiments, both the antigen and the plant lectin or mimetic thereof are embedded in or attached to the surface of the micelle.

The antigen and/or the plant lectin or mimetic thereof may be associated with the micelle using any suitable means known in the art. For example, they may be encapsulated by the micelle as it forms, embedded in the surface of the micelle (e.g., a hydrophobic portion of the antigen may be embedded in the hydrophobic region of the surfactant bilayer whilst a hydrophilic portion of the antigen extends outwardly from the surface of the micelle) or attached to the surface of the micelle. They may be attached to the surface of the micelle directly (e.g., they may be adsorbed to the surface of the micelle or they may form a covalent or non-covalent bond with the surface of the micelle) or indirectly (i.e., one or more linker molecules may be interposed between the surface of the micelle and the antigen and/or the plant lectin or mimetic thereof).

In some embodiments, the antigen is encapsulated within the lumen of a micelle as it forms and the plant lectin or mimetic thereof is embedded in or attached (either directly or indirectly) to the surface to the micelle. In some embodiments, both the antigen and the plant lectin or mimetic thereof are embedded in or attached (either directly or indirectly) to the surface of the micelle. For example, an antigen may be adsorbed to the surface of the micelle whilst UEA-1 or a mimetic thereof is attached to the micelle via a linker molecule embedded in the surfactant bilayer.

In some embodiments, an antigen and/or a plant lectin or a mimetic thereof is conjugated to an individual surfactant molecule and combined into a self-assembling spheroid particle. In some embodiments, both the antigen and the plant lectin or mimetic thereof are conjugated to surfactant molecules and combined into a self-assembling spheroid particle. For example, an antigen and UEA-1 or a mimetic thereof may each be conjugated to a distinct surfactant molecule and then mixed with a sufficient number of additional surfactant molecules to form a micelle comprising the antigen and UEA-1 or the mimetic thereof.

Particles

The antigen and/or the plant lectin or mimetic thereof may be associated with a particle. In some embodiments, the antigen is contained within the particle. In some embodiments, the antigen and/or the plant lectin or mimetic thereof is embedded in or attached to the surface of the particle. In some embodiments, both the antigen and the plant lectin or mimetic thereof are embedded in or attached to the surface of the particle.

Any suitable particle may be used in compositions of the present invention, including, but not limited to, metallic oxide particles, biocompatible polymer particles, solid lipid particles, polymer-coated nanoparticles, poly(methyl methacrylate) particles, poly(alkyl cyanoacrylate) particles, polyacrylate particles, PS particles, PGA particles, PLA particles, PLGA particles, carboxylated and poly(ethylene glycol)-functionalised PLGA nanoparticles and stearic acid-conjugated pullulan (SAP) particles. See generally U.S. Patent Publication Nos. 2004/0022840 and 2007/0237826; Farokhzad et al., PROC. NATL. ACAD. SCI. USA 10:1073 (2006); Kim and Oh, ARCH. PHARM. RES. 33:761-767 (2010); Kreuter, J. ANAT. 189:503 (1996); Kwon et al. COLLOID POLYM. SCI. 286:1181 (2008). In some embodiments, the particles are microparticles or nanoparticles.

Particles may be synthesized via any suitable method known in the art. See, e.g., U.S. Patent Publication Nos. 2004/0022840 and 2007/0237826; Kreuter, J. ANAT. 189:503 (1996).

The antigen and/or the plant lectin or mimetic thereof may be associated with the particle using any suitable means known in the art. See, e.g., U.S. Patent Publication Nos. 2004/0022840 and 2007/0237826. For example, they may be embedded in the surface of the particle (e.g., a portion of the antigen may be embedded in the particle whilst a portion of the antigen extends outwardly from the surface of the particle) or attached to the surface of the particle. They may be attached to the surface of the particle directly (e.g., they may be adsorbed to the surface of the particle or they may form a covalent or non-covalent bond with the surface of the particle) or indirectly (i.e., one or more linker molecules may be interposed between the surface of the particle and the antigen and/or the plant lectin or mimetic thereof).

In some embodiments, both the antigen and the plant lectin or mimetic thereof are adsorbed to, embedded in or attached (either directly or indirectly) to the surface of the particle. For example, an antigen may be adsorbed to the surface of the particle whilst UEA-1 or a mimetic thereof is attached to the particle via a linker molecule that is embedded in or attached to the surface of the particle.

In some embodiments, the antigen and/or the plant lectin or mimetic thereof is attached to the surface of the particle via a linker that ensures that the antigen and/or the plant lectin or mimetic thereof is attached to the particle in a desired orientation (e.g., with a particular epitope extending outwardly from the surface of the particle). For example, a heterobifunctional linker (e.g., hydrazide-polyethylene glycol-dithiol) may be used to attach an antigen and/or a plant lectin or mimetic thereof to a gold nanoparticle in an orientation that maximizes their efficacy (e.g., an antigen may be attached to the particle with a target epitope extending outwardly from the surface of the particle). See generally Kumar and Sokolov, NATURE PROTOCOLS 3:314-320 (2008). As one of skill in the art will appreciate, variations in the orientation of the antigen(s) and/or plant lectin(s) or mimetic(s) thereof may facilitate cell-type-specific targeting (e.g., a plant lectin having a first epitope that targets a first cell type and a second epitope that targets a second cell type may be used to selectively target the second cell type by orienting the plant lectin on the particle in an orientation that diminishes/eliminates the targeting effects of the first epitope and/or that enhances/maximizes the targeting effects of the second epitope).

In some embodiments, the particle is coated with one member of a binding pair and an antigen and/or a plant lectin or a mimetic thereof is conjugated with a corresponding member of the binding pair. The antigen and/or plant lectin or mimetic thereof is attached to the surface of the particle via an interaction between the two members of the binding pair. For example, the particle may be coated with streptavidin or avidin, and a biotinylated antigen and/or a biotinylated plant lectin or a mimetic thereof may be attached to the surface of the particle via an interaction between the attached biotin and the streptavidin/avidin coating on the particle. Alternatively, the particle may be coated with a chelating compound (e.g., nickel-nitroacetic acid), and a His-tagged antigen and/or a His-tagged plant lectin or a mimetic thereof may be attached to the surface of the particle via an interaction between the His-tag and the chelating compound.

### Methods

The present invention also provides methods of using a composition comprising an antigen and a plant lectin or a mimetic thereof. In some embodiments, methods of the present invention comprise administering to a subject a conjugate comprising an antigen and a plant lectin or a mimetic thereof. Any suitable antigen may be used in methods of the present invention (see discussion above with respect to compositions of the present invention).

Methods of the present invention may comprise vaccinating and/or treating a subject. In some embodiments, methods of the present invention may comprise vaccinating a subject with an antigen. In some embodiments, methods of the present invention may comprise treating a subject for a disorder.

Methods of the present invention may be used to elicit an enhanced immune response. In some embodiments, methods of the present invention may be used to elicit an enhanced cellular immune response without eliciting an enhanced humoral immune response (e.g., in a subject in need of an enhanced cellular immune response in the absence of an enhanced humoral immune response). In some embodiments, the immune response enhanced is a protective and/or a therapeutic immune response.

Targeting and/or Delivering an Antigen

One aspect of the present invention is a method of targeting and/or delivering an antigen to leukocytes in a subject, which may comprise, consist essentially of or consist of administering to the subject a composition comprising the antigen and a plant lectin or a mimetic thereof. In some embodiments, the method comprises, consists of or consists essentially of administering to the subject a composition of the present invention. In some embodiments, the method comprises, consists of or consists essentially of administering to the subject a conjugate comprising the antigen and a plant lectin or mimetic thereof.

Another aspect of the present invention is a method of targeting and/or delivering an antigen to leukocytes in vitro or ex vivo, which may comprise, consist essentially of or consist of contacting the leukocytes with a medium comprising the antigen and a plant lectin or a mimetic thereof. In some embodiments, the method comprises, consists of or consists essentially of contacting the leukocytes with a medium comprising a composition of the present invention. In some embodiments, the method comprises, consists of or consists essentially of contacting the leukocytes with a medium comprising a conjugate comprising the antigen and a plant lectin or mimetic thereof.

Such methods may result in an increase in the number of leukocytes taking up the antigen and/or an increase in the amount of antigen taken up per leukocyte (as compared to a method wherein leukocytes are contacted with a composition lacking a plant lectin or a mimetic thereof, for example).

The antigen may be targeted and/or delivered to any suitable leukocyte(s), including, but not limited to, granulocytes (including neutrophils, basophils and eosinophils), lymphoblasts, B cells, monocytes, macrophages, dendritic cells and microglia. In some embodiments, the leukocytes are leukocytes other than T cells. In some embodiments, the leukocytes are antigen-presenting cells. In some embodiments, the leukocytes are phagocytic cells. In some embodiments, the leukocytes are selected from the group consisting of dendritic cells, monocytes and granulocytes. In some embodiments, the leukocytes are dendritic cells.

The antigen may be targeted and/or delivered to one or more leukocytes in the absence of targeting to microfold cells (M cells).

Increasing Antigen Uptake

Another aspect of the present invention is a method of increasing the uptake of an antigen by leukocytes in a subject, which may comprise, consist essentially of or consist of administering to the subject a composition comprising the antigen and a plant lectin or a mimetic thereof. In some embodiments, the method comprises, consists of or consists essentially of administering to the subject a composition of the present invention. In some embodiments, the method comprises, consists of or consists essentially of administering to the subject a conjugate comprising the antigen and a plant lectin or mimetic thereof.

Another aspect of the present invention is a method of increasing the uptake of an antigen by leukocytes in vitro or ex vivo, which may comprise, consist essentially of or consist of contacting the leukocytes with a medium comprising the antigen and a plant lectin or a mimetic thereof. In some embodiments, the method comprises, consists of or consists essentially of contacting the cells with a medium comprising a composition of the present invention. In some embodiments, the method comprises, consists of or consists essentially of contacting the leukocytes with a medium comprising a conjugate comprising the antigen and a plant lectin or mimetic thereof.

Such methods may result in an increase in the number of leukocytes taking up the antigen and/or an increase in the amount of antigen taken up per leukocyte (as compared to a method wherein leukocytes are contacted with a composition lacking a plant lectin or a mimetic thereof, for example).

These methods may be used to increase the uptake of an antigen by any suitable leukocyte(s), including, but not limited to, granulocytes (including neutrophils, basophils and/or eosinophils), lymphoblasts, B cells, monocytes, macrophages, dendritic cells and/or microglia. In some embodiments, the leukocytes are leukocytes other than T cells. In some embodiments, the leukocytes are antigen-presenting cells. In some embodiments, the leukocytes are phagocytic cells. In some embodiments, the leukocytes are selected from the group consisting of dendritic cells, monocytes and granulocytes. In some embodiments, the leukocytes are dendritic cells.

Stimulating a T_{H}1 and/or a T_{H}17 Response

Another aspect of the present invention is a method of stimulating a T_{H}1 and/or a T_{H}17 response in a subject, which may comprise, consist essentially of or consist of administering to the subject a composition comprising an antigen and a plant lectin or a mimetic thereof. In some embodiments, the method comprises, consists of or consists essentially of administering to the subject a composition of the present invention. In some embodiments, the method comprises, consists of or consists essentially of administering to the subject a conjugate comprising the antigen and a plant lectin or mimetic thereof.

Without wishing to be bound by any particular theory, it is currently believed that compositions of the present invention stimulate T_{H}1 and/or T_{H}17 responses by contacting one or more suitable leukocyte(s), including, but not limited to, granulocytes (including neutrophils, basophils and/or eosinophils), lymphoblasts, B cells, monocytes, macrophages, dendritic cells and/or microglia. In some embodiments, the leukocytes are leukocytes other than T cells. In some embodiments, the leukocytes are antigen-presenting cells. In some embodiments, the leukocytes are phagocytic cells. In some embodiments, the leukocytes are selected from the group consisting of dendritic cells, monocytes and granulocytes. In some embodiments, the leukocytes are dendritic cells.

Enhancing an Immune Response

Another aspect of the present invention is a method of enhancing an immune response to an antigen in a subject, which may comprise, consist essentially of or consist of administering to the subject a composition comprising an antigen and a plant lectin or a mimetic thereof. In some embodiments, the method comprises, consists of or consists essentially of administering to the subject a composition of the present invention. In some embodiments, the method comprises, consists of or consists essentially of administering to the subject a conjugate comprising the antigen and a plant lectin or mimetic thereof.

The immune response enhanced may comprise a cellular immune response and/or a humoral immune response. In some embodiments, a cellular immune response is enhanced in the absence of an enhanced humoral immune response.

The immune response enhanced may comprise a protective immune response and/or a therapeutic immune response. For example, methods of the present invention may be used to enhance the efficacy of a vaccine and/or to enhance an immune response against a particular cancer antigen.

Any suitable route of administration may be used in methods of the present invention including, but not limited to, oral, intravenous (i.v.), subcutaneous, intramuscular, intrathecal, intraperitoneal (i.p.), intrarectal, intravaginal, intranasal, intragastric, intratracheal, transcutaneous, sublingual and intrapulmonary. In some embodiments, a composition of the present invention is administered to a subject via a non-oral route of administration (e.g., intraperitoneal injection or intranasal administration).

The dosage required for methods of the present invention may depend on numerous factors, including, but not limited to, the route of administration, the identity of the antigen, the identity of the plant lectin or mimetic thereof, the presence/absence of adjuvant, the age/sex/weight/surface area of the subject and the presence/absence of other drugs/illnesses/allergies. Variations in dosage levels may be adjusted using standard empirical routines for optimization, as is well understood in the art.

### EXAMPLES

The following examples are not intended to be a detailed catalogue of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### Example 1

### Materials and Methods

Animals

Pathogen-free female C3H/HeN, C3H/HeJ, BALB/c, C57BL/6 and NLRP3^{-/-} mice were maintained according to the regulations and guidelines of the European Union and the Irish Department of Health. All experiments were conducted under university ethical approval and under license from the Department of Health and Children. Mice were 6-8 weeks old at the initiation of each experiment.

UEA-1

UEA-1 and biotinylated UEA-1 were obtained from Vector Laboratories Ltd. (Peterborough, England, UK). Lectins were dissolved in 2 ml of sterile H₂O to a final concentration of 2 mg/ml and stored at 4° C.

UEA-1 Mimetic

UEA-1 mimetic and biotinylated UEA-1 mimetic was obtained from PolyPeptide Laboratories (San Diego, Calif.). Mimetic was dissolved in 600 μl DMSO and 400 μl Dulbecco's PBS to a final concentration of 4.3 mg/ml and stored at 4° C.

Additional Plant Lectins

Biotinylated Con A, biotinylated DBA, biotinylated DSL, biotinylated GSL I, biotinylated GSL II, biotinylated Jac, biotinylated LEL, biotinylated PHA-E, biotinylated PHA-L, biotinylated PNA, biotinylated PSA, biotinylated SBA, biotinylated VVL and biotinylated WGA were obtained from Vector Laboratories Ltd. (Peterborough, England, UK). The biotinylated lectins were dissolved in 500 μl sterile H₂O to prepare a final concentration of 2 mg/ml and stored at 4° C.

Polystyrene Particles

Polystyrene (PS) particles (430 nm; 10 mg/ml), streptavidin-coated polystyrene (SC-PS) particles (300-430 nm; 10 mg/ml) and Nile Red streptavidin-coated polystyrene (NR-PS) particles (400-600 nm; 10 mg/ml) were stored at 4° C.

Chitosan

Protasan™ Ultrapure CL213 chitosan was obtained from NovaMatrix™ (Sandvika, Norway).

Alum

Alhydrogel™ (Brenntag Biosector, Frederiksund, Denmark) was stored at 4° C.

Complete RPMI

40 ml sterile-filtered, heat-inactivated (56° C. for 30 min) foetal calf serum (FCS), 5 ml antibiotics (100 μg/ml streptomycin and 100 U/ml penicillin) and 5 ml 100 mM L-glutamine were added to 500 ml Roswell Park Memorial Institute 1640 medium.

Attachment Buffer

4.9 ml of a sodium phosphate monobasic solution (2.84 g NaH₂PO₄ in 100 ml sterile H₂O) was added to 70.1 ml of a sodium phosphate dibasic solution (2.78 g Na₂HPO₄ in 100 ml sterile H₂O), sterile filtered and adjusted to pH 5.5.

FACS Buffer

10 ml FCS and 0.5 ml sodium azide (10%) were added to 500 ml Dulbecco's PBS.

Pathogen Recognition Receptor Agonists

The following Pathogen Recognition Receptor (PRR) agonists were made up in complete RPMI 1640 medium at the stated concentrations: LPS (1 ng/ml; Toll-like receptor 4 ligand) and Pam2CSK4 (50 ng/ml; Toll-like receptor 1 and Toll-like receptor 2 ligand).

PBS-T

0.05% Tween-20 and 1 L of 10×PBS (400 g NaCl, 58 g Na₂HPO₄, 10 g KH₂PO₄ and 10 g KCl in 5 L dH₂O, adjusted to pH 7.2) were added to 9 L dH₂O.

Substrate Solution

One OPD Tablet (20 mg) and 20 μl H₂O₂ were added to 50 ml of phosphate citrate buffer (10.19 g anhydrous citric acid and 36.9 g Na₂HPO₄ in 1 L dH₂O, adjusted to pH 5).

### Example 2

### Cell Isolation and Culture

All cell culturing and incubation steps were performed in a 37° C. incubator with an atmosphere maintained at 95% humidity and 5% CO₂ (v/v).

Isolation of Bone Marrow-Derived Dendritic Cells

Bone marrow-derived dendritic cells (BMDCs) were generated from C3H/HeN, C3H/HeJ, C57BL/6 or NLRP3^{-/-} mice using a method adapted from Lutz et al. (J. IMMUNOL. METH. 223(1):77 (1999)). Mice were sacrificed by cervical dislocation and their hind legs removed. Both femurs and tibiae were dissected and all surrounding muscle and fatty tissue removed. The tips of the bones were carefully cut at both ends just enough to expose the red bone marrow, which was extracted by the insertion of a bent, sterile 27G needle attached to a syringe containing complete RPMI 1640 medium and flushed out into a sterile petri dish. Cell aggregates were broken up using a 19G needle before being transferred into a sterile 50 ml tube. Cells were pelleted by centrifugation at 1200 rpm for 5 minutes at 20° C. The supernatant was poured off and the pellet was resuspended in 2 ml of cold filter-sterilized ammonium chloride solution (0.88%) to lyse red blood cells. After 2 minutes, 40 ml of complete RPMI 1640 medium was added to the tube and centrifuged as above. Cells were then resuspended in 10 ml of complete RPMI 1640 medium and counted.

Cells were cultured at a density of either 1×10⁶ cells/ml (C3H/HeJ or C3H/HeN) or 4.2×10⁵ cells/ml (C57BL/6 or NLRP3^{-/-}) in T175 tissue culture flasks in complete RPMI 1640 medium containing granulocyte-macrophage colony-stimulating factor (GM-CSF) (20 ng/ml), at a total volume of 30 ml. All flasks were maintained in an incubator at 37° C. in 5% CO₂. Cells were cultured with a further 30 ml of complete RPMI 1640 medium containing GM-CSF (20 ng/ml) on day 3.

On day 6 supernatants were discarded and loosely adherent cells removed by flushing with 30 ml sterile Dulbecco's PBS. This suspension was added to complete RPMI 1640 medium and retained. 20 ml EDTA (0.02%) was then added to the flask and incubated. After 10 minutes, the remaining adherent dendritic cells were removed by flushing with EDTA before being removed and added to complete RPMI 1640 medium. Cells were then centrifuged at 1200 rpm for 5 minutes. Pellets were resuspended in 10 ml of complete RPMI 1640 medium and counted. Cells were re-cultured at a density of 7×10⁵ cells/ml (C3H/HeJ or C3H/HeN) or 4.2×10⁵ cells/ml (C57BL/6 or NLRP3^{-/-}) in fresh T175 tissue culture flasks in 30 ml complete RPMI 1640 medium with GM-CSF (20 ng/ml). On day 8 cells were cultured with an additional 30 ml of complete RPMI 1640 medium with GM-CSF (20 ng/ml).

On day 10 loosely adherent cells were collected by flushing the flasks with the medium. The cell suspension was collected and centrifuged at 1200 rpm for 5 minutes. Cells were resuspended in 10 ml complete RPMI 1640 medium and counted. Cells were then used for stimulations in complete RPMI medium with GM-CSF (10 ng/ml) as detailed in the subsequent experimental sections. Cells were incubated for 1-2 days to allow for cells to adhere to plates before use.

Culture of Bone Marrow-Derived Macrophages

Bone marrow-derived macrophages (iBMMs) are an immortalised cell line. The cells were cultured in complete RPMI 1640 medium in T175 flasks until confluent, and the medium and loosely adherent cells were removed and discarded. 20 ml complete RPMI 1640 was added to the flask, the adherent iBMMs lifted from the flask with a cell scraper, and 2 ml of the cell suspension was transferred to a new flask with 20 ml complete RPMI 1640 medium.

Isolation of Spleen Cells

Mice were sacrificed by cervical dislocation before removal of their spleens. Single cell suspensions were prepared by disrupting tissue through 70 μm nylon cell strainers with complete RPMI 1640 medium. The cells were then centrifuged at 1200 rpm for 5 minutes and the cell pellet resuspended in 1 ml ammonium chloride (0.88%) for 2 minutes. Cells were then washed in complete RPMI 1640 medium and centrifuged again. Cells were then resuspended in 5 ml of complete RPMI 1640 medium and counted. Cells were plated as described in the relevant experimental section.

Collection of Peritoneal Lavage Cells

Peritoneal lavage washes were carried out with 5 ml Dulbecco's PBS, Cells were pelleted by centrifugation at 1200 rpm for 5 minutes. Cells were resuspended in 1 ml of complete RPMI 1640 medium and cell counts performed. Cells were plated as described in the relevant experimental section.

Serum Collection

Blood was collected from the tail veins of mice and allowed to clot overnight at 4° C. Samples were then centrifuged at 5000 rpm for 10 minutes. The serum was separated from the blood cells and stored at −20° C. until further use.

Cell Counting

Cell suspensions were diluted 1:10 (bone marrow-derived dendritic cells) or 1:50 (splenocytes) with Trypan Blue. 10 μl of suspension was added into a cell counter slide and viewed under a light microscope under the ×10 objective lens. The number of viable cells was determined. The concentration of cells (cells/ml) was then calculated using the following formula: cells/ml=cell count×dilution factor (10 or 50)×10⁴.

### Example 3

### Preparation of UEA-1 Conjugates

Adsorption of UEA-1 to PS Particles and Alum

PS particles were centrifuged at 14,000 rpm at 4° C. for 10 minutes. The supernatant was removed and replaced with Dulbecco's PBS. This particle preparation was transferred to a 5 ml tube, to which 100 μg/ml of UEA-1 was added and made up to a final volume of at least 500 μl with Dulbecco's PBS to ensure proper mixing. The mixture was incubated for 1.5 hours, rotating at room temperature. The mixture was again centrifuged at 14,000 rpm at 4° C. for 10 minutes. The supernatant was removed and a BCA™ protein assay performed to determine the amount of UEA-1 attached to the particles. The particles were resuspended in complete RPMI medium. This stock was then diluted further with complete RPMI 1640 medium to achieve required concentrations. An identical method was used to adsorb UEA-1 to alum.

Conjugation of Biotinylated UEA-1 to SC-PS Particles

SC-PS particles were centrifuged at 14,000 rpm at 4° C. for 10 minutes. The supernatant was removed and replaced with sterile attachment buffer. This particle preparation was transferred to a separate 5 ml tube, to which 100 μg/ml of biotinylated UEA-1 was added and made up to a final volume of at least 500 μl with sterile attachment buffer to ensure proper mixing. The mixture was incubated for 1 hour, rotating at room temperature. The mixture was again centrifuged at 14,000 rpm at 4° C. for 10 minutes. The supernatant was removed and a BCA™ protein assay performed to determine the amount of biotinylated UEA-1 attached to the particles. The particles were resuspended in complete RPMI 1640 medium. This stock was then diluted further with complete RPMI 1640 medium to achieve required concentrations.

Conjugation of Biotinylated UEA-1 Mimetic to SC-PS Particles

SC-PS particles were centrifuged at 14,000 rpm at 4° C. for 10 minutes. The supernatant was removed and replaced with sterile attachment buffer. This particle preparation was transferred to a separate 5 ml tube, to which 100 μg/ml of biotinylated UEA-1 mimetic was added and made up to a final volume of at least 500 μl with sterile attachment buffer to ensure proper mixing. The mixture was incubated for 1 hour, rotating at room temperature. The mixture was again centrifuged at 14,000 rpm at 4° C. for 10 minutes. The supernatant was removed and a BCA™ protein assay performed to determine the amount of biotinylated UEA-1 mimetic attached to the particles. The particles were resuspended in complete RPMI 1640 medium. This stock was then diluted further with complete RPMI 1640 medium to achieve required concentrations.

Conjugation of Additional Biotinylated Lectins to SC-PS Particles

SC-PS particles were centrifuged at 14,000 rpm at 4° C. for 10 minutes. The supernatant was removed and replaced with sterile attachment buffer to bring the particle concentration to 1% w/v. 100 μg/ml of biotinylated Con A, biotinylated DBA, biotinylated DSL, biotinylated GSL I, biotinylated GSL II, biotinylated Jac, biotinylated LEL, biotinylated PHA-E, biotinylated PHA-L, biotinylated PNA, biotinylated PSA, biotinylated SBA, biotinylated VVL or biotinylated WGA were added to the particles. The mixture was incubated for 1 hour at room temperature with regular mixing. The mixture was again centrifuged at 14,000 rpm at 4° C. for 10 minutes. The supernatant was removed and a BCA™ protein assay performed to determine the amount of biotinylated lectin attached to the particles. The particles were resuspended in complete RPMI 1640 medium. This stock was then diluted further with complete RPMI 1640 medium to achieve required concentrations.

Conjugation of Biotinylated UEA-1 to NR-PS Particles

NR-PS particles were centrifuged at 14,000 rpm at 4° C. for 10 minutes. The supernatant was removed and replaced with sterile attachment buffer. This particle preparation was transferred to a separate 5 ml tube, to which 100 μg/ml of biotinylated UEA-1 was added and made up to a final volume of at least 500 μl with sterile attachment buffer to ensure proper mixing. The mixture was incubated for 1 hour, rotating at room temperature. The mixture was again centrifuged at 14,000 rpm at 4° C. for 10 minutes. The supernatant was removed and a BCA™ protein assay performed to determine the amount of biotinylated UEA-1 attached to the particles. The particles were resuspended in complete RPMI 1640 medium. This stock was then diluted further with complete RPMI 1640 medium to achieve required concentrations.

Conjugation of Biotinylated UEA-1 Mimetic to NR-PS Particles

NR-PS particles were centrifuged at 14,000 rpm at 4° C. for 10 minutes. The supernatant was removed and replaced with sterile attachment buffer. This particle preparation was transferred to a separate 5 ml tube, to which 100 μg/ml of biotinylated UEA-1 mimetic was added and made up to a final volume of at least 500 μl with sterile attachment buffer to ensure proper mixing. The mixture was incubated for 1 hour, rotating at room temperature. The mixture was again centrifuged at 14,000 rpm at 4° C. for 10 minutes. The supernatant was removed and a BCA™ protein assay performed to determine the amount of biotinylated UEA-1 mimetic attached to the particles. The particles were resuspended in complete RPMI 1640 medium. This stock was then diluted further with complete RPMI 1640 medium to achieve required concentrations.

Conjugation of Additional Biotinylated Lectins to NR-PS Particles

NR-PS particles were centrifuged at 14,000 rpm at 4° C. for 10 minutes. The supernatant was removed and replaced with sterile attachment buffer to bring the particle concentration to 1% w/v. 100 μg/ml of biotinylated DSL, biotinylated PHA-E, biotinylated PHA-L or biotinylated SBA were added to the particles. The mixture was incubated for 1 hour, rotating at room temperature. The mixture was again centrifuged at 14,000 rpm at 4° C. for 10 minutes. The supernatant was removed and a BCA™ protein assay performed to determine the amount of biotinylated lectin attached to the particles. The particles were resuspended in complete RPMI 1640 medium. This stock was then diluted further with complete RPMI 1640 medium to achieve required concentrations.

BCA™ Protocol to Measure Lectin Attachment

A BCA™ Protein Assay (Pierce Biotechnology, Rockford, Ill.) was used to determine the amount of lectin/mimetic attached to the particles. The amount of lectin/mimetic attached to the particles was calculated by subtracting the amount of lectin/mimetic in the supernatant from the initial amount of lectin/mimetic added to the particle preparation. 25 μl of the standards and the samples were added in triplicate to a 96 well medium affinity ELISA plate. The BCA™ assay mixture was prepared by adding 100 μl of BCA™ Reagent B to 5000 μl of BCA™ Reagent A (1:50). 200 μl of the mixed BCA™ assay mixture was then added to each well. Samples were incubated at 37° C. for 30 mins in the dark, with light rocking. The absorbance was measured at a wavelength of 562 nm and analyzed using a VersaMax™ microplate reader (Molecular Devices, Inc., Sunnyvale, Calif.) and SoftMax® Pro Data Acquisition & Analysis Software (Molecular Devices, Inc., Sunnyvale, Calif.). Unknown protein concentrations were determined by extrapolating from a standard curve.

### Example 4

### UEA-1 Targeting Increases Particle Uptake by Dendritic Cells

C57BL/6 BMDCs were cultured onto sterile glass 19 mm cover slips in a 12 well plate at a density of 1×10⁶ cells/ml in 2 ml complete RPMI 1640 medium with GM-CSF (10 ng/ml) and incubated at 37° C. to allow cells to adhere overnight. Surrounding empty wells were filled with Dulbecco's Sterile PBS to prevent dehydration of the wells containing cells. On the following day, the medium was carefully removed and replaced with 500 μl of complete RPMI 1640 medium with NR-PS particles (1.0 mg/ml or 200 μg/ml) or NR-PS particles (1.0 mg/ml or 200 μg/ml) conjugated with biotinylated UEA-1 (100 μg/ml). These were incubated for 1 hour. After incubation, the cells were washed with 1×PBS and fixed in 2% formaldehyde in 1×PBS for 30 minutes at room temperature, then washed 3 times with 1×PBS. Cell membranes were stained with 250 μl Alexa Fluor® 488 Phalloidin (Invitrogen Life Sciences, Carlsbad, Calif.) diluted 1:50 in 1×PBS at room temperature, for 3 hours in the dark. Three subsequent washes with 1×PBS were performed. Cell nuclei were stained with a DAPI nucleic acid stain diluted 1:1000 in 1×PBS for 5 minutes in the dark at room temperature, after which a further 3 washes with 1×PBS were performed. Cover slips were carefully removed from the wells and washed in dH₂O. The edges of the cover slips were dabbed on a paper towel to dry them. The cover slips were mounted on glass slides in a drop of fluorescent mounting medium, cell side down. Slides were viewed using a FluoView™ 1000 confocal microscope (Olympus, Center Valley, Pa.) under the oil emersion objective.

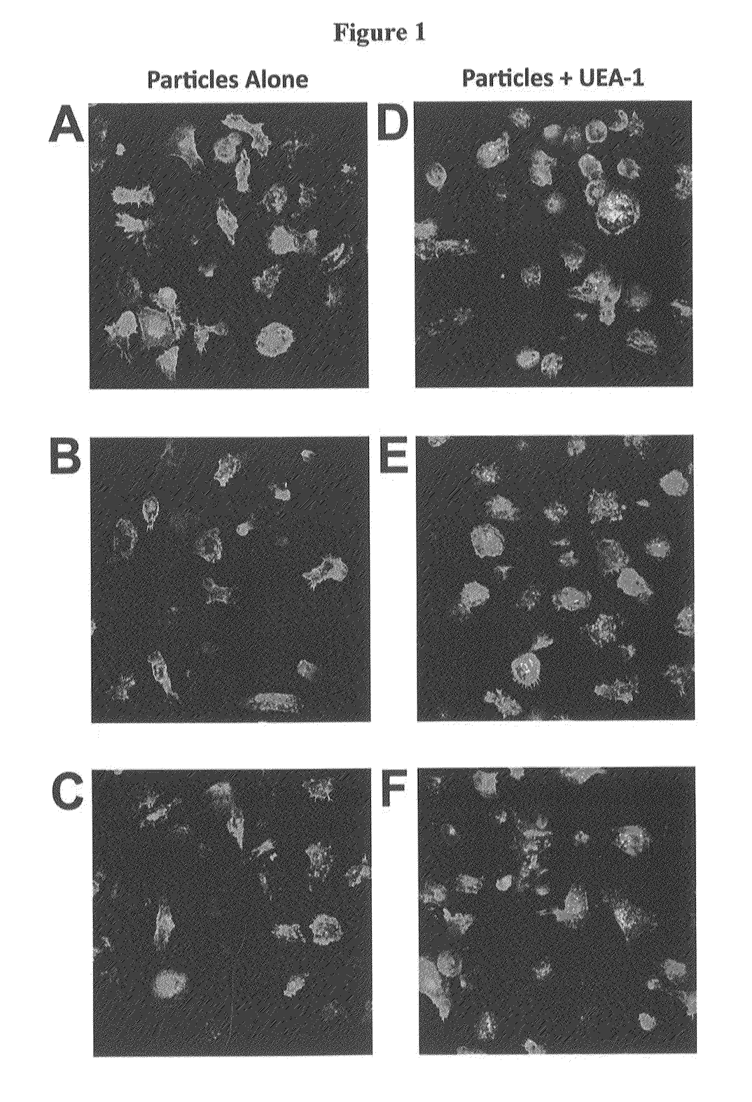

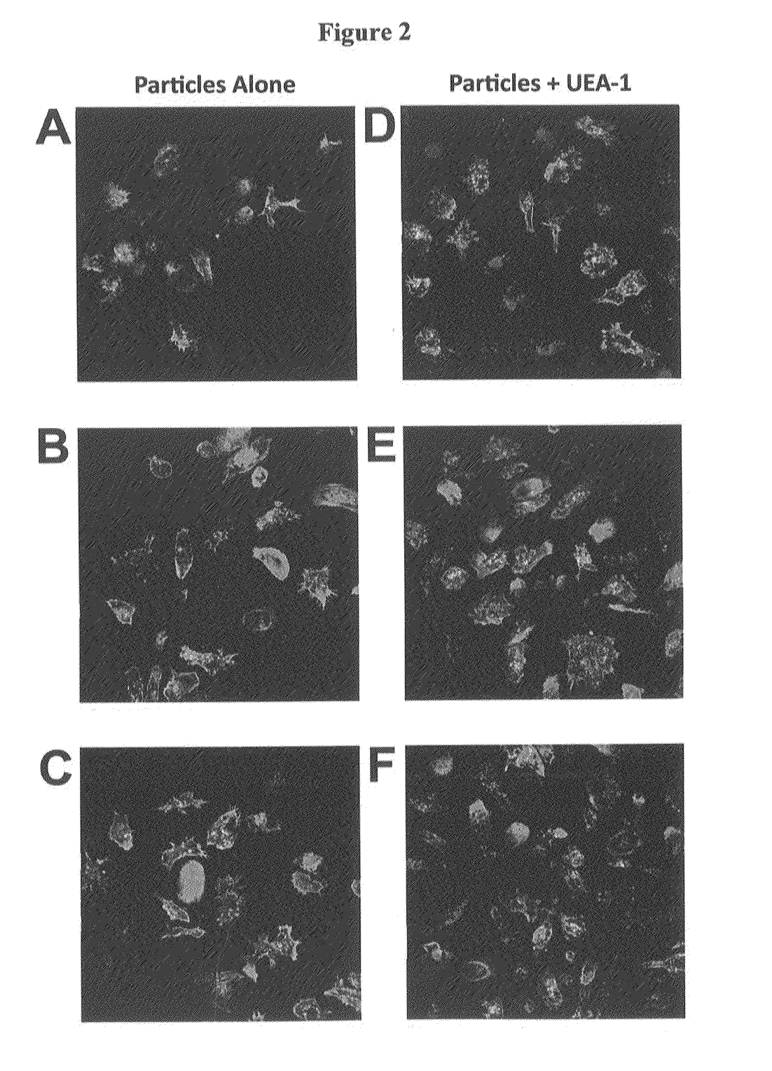

As shown in FIG. 1 and FIG. 2, conjugating the NR-PS particles with biotinylated UEA-1 increases both the number of dendritic cells taking up particles and the number of particles taken up per cell. In each of the aforementioned figures, cells incubated with NR-PS particles conjugated with biotinylated UEA-1 (D-F) take up more particles than cells incubated with unconjugated NR-PS (A-C). Interestingly, reducing the concentration of the particles from 1.0 mg/ml (FIG. 1) to 200 μg/ml (FIG. 2) increased both the number of cells taking up unconjugated NR-PS and the number of unconjugated NR-PS particles taken up per cell. The contrast between conjugated (D-F) and unconjugated (A-C) NR-PS particles also substantially increased when the particle concentration was reduced. That is, UEA-1 targeting increased the number of dendritic cells taking up particles and the number of particles taken up per cell more markedly when the cells were incubated with 200 μg/ml of nanoparticles conjugated with 100 μg/ml of biotinylated UEA-1, as compared to 1 mg/ml of conjugated particles.

Interestingly there are more nuclei present without membranes in the slides containing the higher concentration of UEA-1 targeted particles (FIGS. 1D-1F) which is not visible in the lower amount of targeted PS particles (FIGS. 2D-2F). This could be an indicator of cell lysis.

Thus, conjugating biotinylated UEA-1 to particles appears to target the particles to dendritic cells, increasing both the number of cells taking up particles and the number of particles taken up per cell.

### Example 5

### Quantification of Dendritic Cell Uptake of Particles Conjugated with UEA-1

C57BL/6 BMDCs were isolated and cultured as described above in a 96 well U-bottomed plate in 100 μl complete RPMI 1640 medium with 10 ng/ml GM-CSF. Cells were stimulated for 1 or 2 hours with NR-PS particles (1.0 mg/ml or 200 μg/ml) or NR-PS particles (1.0 mg/ml or 200 μg/ml) conjugated with biotinylated UEA-1 (100 μg/ml). Cells were then scraped into FACS tubes, washed in FACS buffer, centrifuged at 1200 rpm for 5 minutes (×3) and resuspended in 200 μl ft of FACS buffer.

A FACSCalibur™ flow cytometer (BD Biosciences, San Jose, Calif.), CellQuest™ software (BD BioSciences, San Jose, Calif.) and FlowJo™ software (Treestar, Inc., Ashland, Oreg.) were used to analyze the uptake of particles by various cell populations. Particle uptake was quantified by determining the percentage of cells taking up particles and by determining the mean fluorescence intensity (MFI), which represents the number of particles taken up per cell.

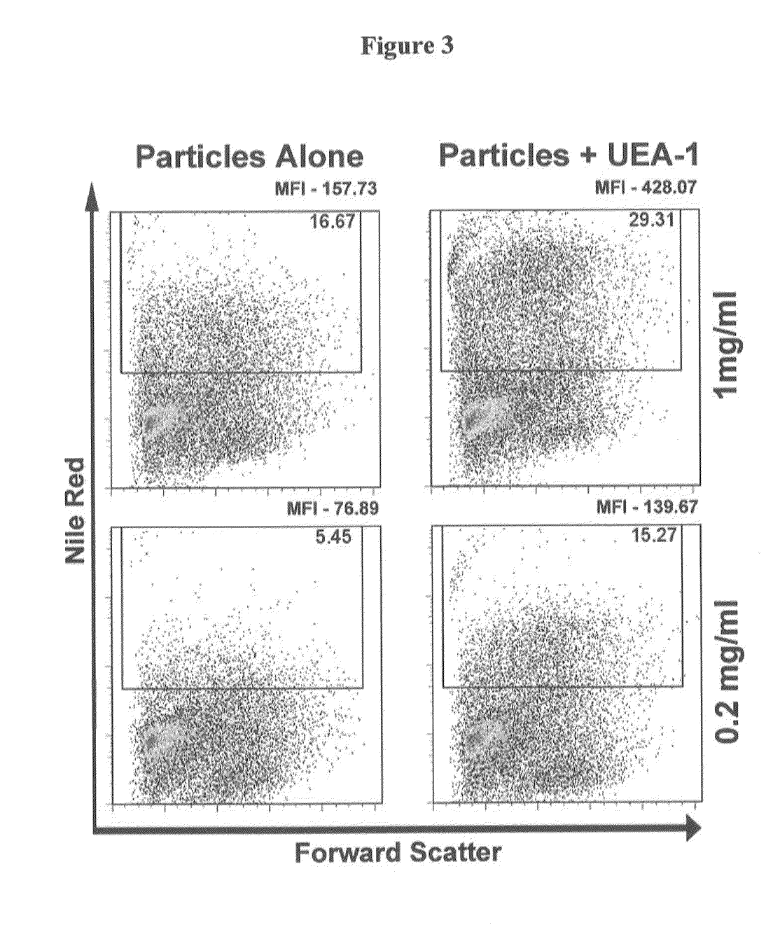

As shown in FIG. 3, 29.31% of the cells were found to have taken up NR-PS particles conjugated with biotinylated UEA-1 after a 1 hour incubation with 1.0 mg/ml of particles, whereas only 16.67% of the cells were found to have taken up unconjugated NR-PS particles under similar conditions. In other words, conjugation with biotinylated UEA-1 increased the percentage of cells taking up particles by 75.82%. Conjugation with biotinylated UEA-1 also increased the number of particles taken up per cell, as evidenced by a nearly three-fold increase in the MFI.

Similar increases were seen when the concentration of particles was reduced to 200 μg/ml of particles. As shown in FIG. 3, 15.27% of the cells were found to have taken up NR-PS particles conjugated with biotinylated UEA-1 after a 1 hour incubation with 200 μg/ml of particles, whereas only 5.45% of the cells were found to have taken up unconjugated NR-PS particles under similar conditions. In other words, conjugation with biotinylated UEA-1 increased the percentage of cells taking up particles by 180.18%. Conjugation with biotinylated UEA-1 also increased the number of particles taken up per cell, as evidenced by a nearly two-fold increase in the MFI (see Table 1).

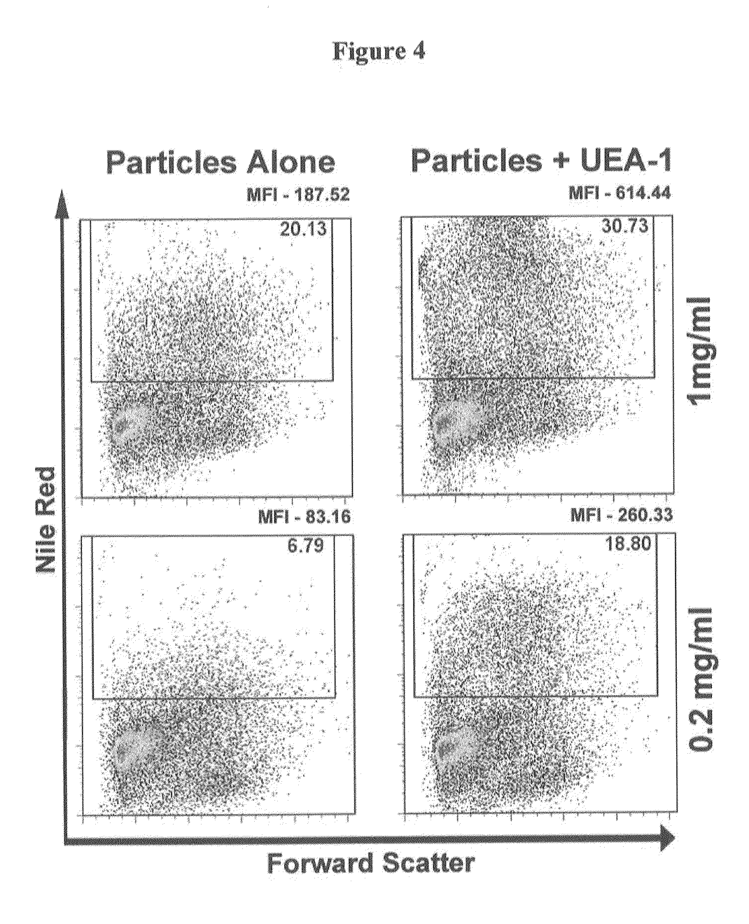

As shown in FIG. 4, increases in both the percentages of cells taking up particles and the number of particles taken up per cell were maintained over the longer incubation period of 2 hours. At 1.0 mg/ml, 30.73% of the cells were found to have taken up NR-PS particles conjugated with biotinylated UEA-1, whereas only 20.13% of the cells were found to have taken up unconjugated NR-PS particles. At 200 μg/ml, 18.8% of the cells were found to have taken up NR-PS particles conjugated with biotinylated UEA-1, whereas only 6.79% of the cells were found to have taken up unconjugated NR-PS particles. In other words, conjugation with biotinylated UEA-1 increased the percentage of cells taking up particles by 52.66% at 1.0 mg/ml and 176.87% at 200 μg/ml. Conjugation with biotinylated UEA-1 also increased the number of particles taken up per cell, as evidenced by a roughly three-fold increase in MFI at both 1.0 mg/ml and 200 μg/ml (see Table 1).

Thus, conjugating biotinylated UEA-1 to particles appears to target the particles to dendritic cells, resulting in large increases in both the number of cells taking up particles and the number of particles taken up per cell (at both the 1 hour and 2 hour time points, and at both the higher and lower particle concentrations).

TABLE 1
Nile red Median Fluorescence Intensity

| Sample | Time (hrs) | MFI |
|---|---|---|
| Control | 2 | 57.97 |
| NR-PS Particles (1.0 mg/ml) | 1 | 157.73 |
| NR-PS Particles + UEA-1 (1.0 mg/ml) | | 428.07 |
| NR-PS Particles (0.2 mg/ml) | | 76.89 |
| NR-PS Particles + UEA-1 (0.2 mg/ml) | | 139.67 |
| NR-PS Particles (1.0 mg/ml) | 2 | 187.52 |
| NR-PS Particles + UEA-1 (1.0 mg/ml) | | 614.44 |
| NR-PS Particles (0.2 mg/ml) | | 83.16 |
| NR-PS Particles + UEA-1 (0.2 mg/ml) | | 260.33 |

### Example 6

### Plant Lectins Effectively Target Particles to BMDCs and iBMMs

C57BL/6 BMDCs and iBMMs were isolated and cultured as described above at a density of 1×10⁶ cells/ml in a 96 well U-bottomed plate in 100 μl complete RPMI 1640 medium with 10 ng/ml GM-CSF. Cells were stimulated for 10 or 30 minutes at 37° C. with NR-PS particles (5, 50 or 100 μg/ml) or NR-PS particles (5, 50 or 100 μg/ml) conjugated with biotinylated UEA-1 (100 μg/ml), biotinylated SBA (100 μg/ml), biotinylated PHA-E (100 μg/ml), biotinylated PHA-L (100 μg/ml) or biotinylated DSL (100 μg/ml). Cells were then scraped into FACS tubes, washed in FACS buffer, centrifuged at 1200 rpm for 5 minutes (×3) and resuspended in 200 μl of FACS buffer.

A FACSCanto™ II flow cytometer (BD Biosciences, San Jose, Calif.), FACSDiva™ software (BD Biosciences, San Jose, Calif.) and FlowJo™ software (Treestar, Inc., Ashland, Oreg.) were used to analyze the uptake of particles by various cell populations. Live cells were gated on by their FSC and SSC properties in order to estimate the degree of cell death. Particle uptake was quantified by determining the percentage of live cells taking up particles. Unstimulated cells were used as controls.

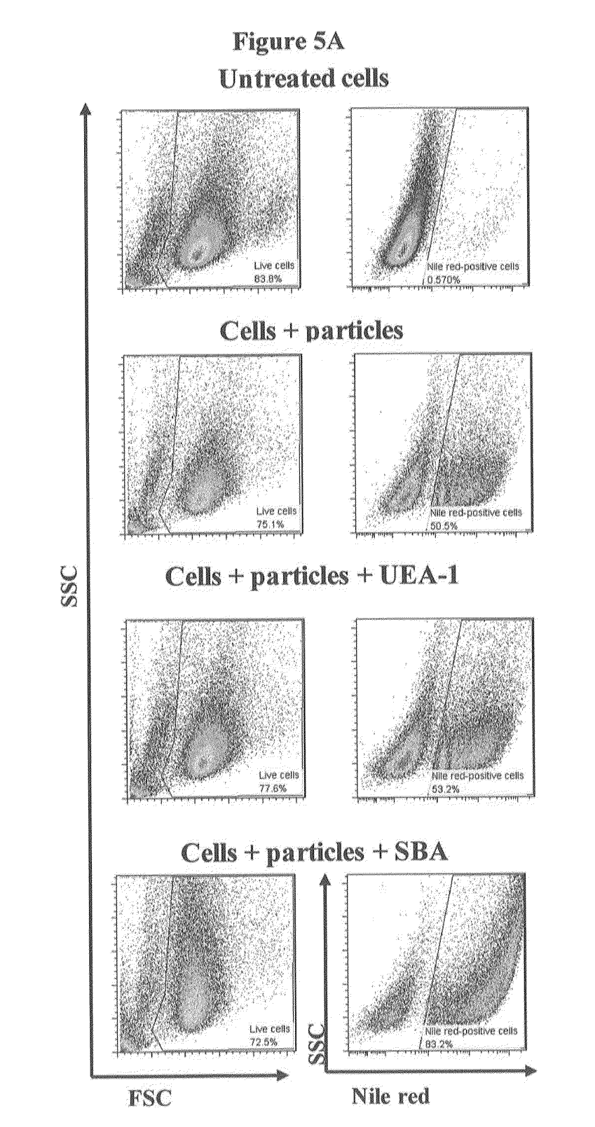

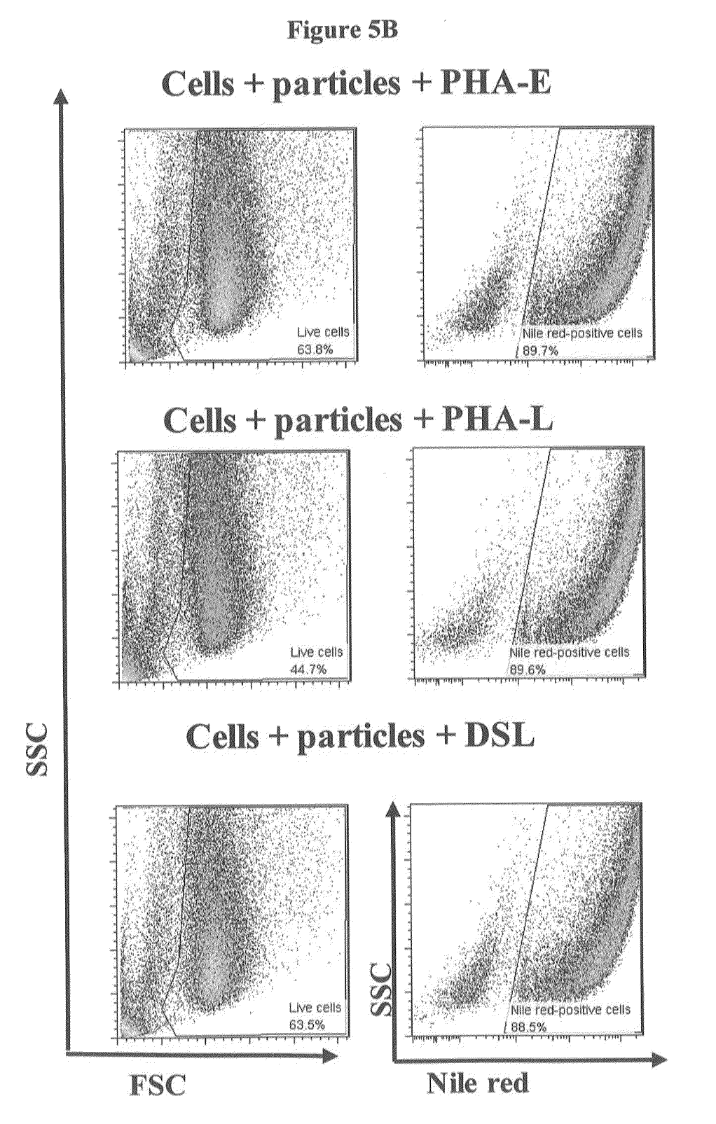

As shown in FIGS. 5A-5B, following a 30 minute incubation, 53.2% of the live BMDCs were found to have taken up NR-PS particles conjugated with biotinylated UEA-1 (100 μg/ml), 83.2% of the live BMDCs were found to have taken up NR-PS particles conjugated with biotinylated SBA (100 μg/ml), 89.7% of the live BMDCs were found to have taken up NR-PS particles conjugated with biotinylated PHA-E (100 μg/ml), 89.6% of the live BMDCs were found to have taken up NR-PS particles conjugated with biotinylated PHA-L (100 μg/ml) and 88.5% of the live BMDCs were found to have taken up NR-PS particles conjugated with biotinylated DSL (100 μg/ml), whereas only 50.5% of the live BMDCs were found to have taken up unconjugated NR-PS particles under similar conditions.

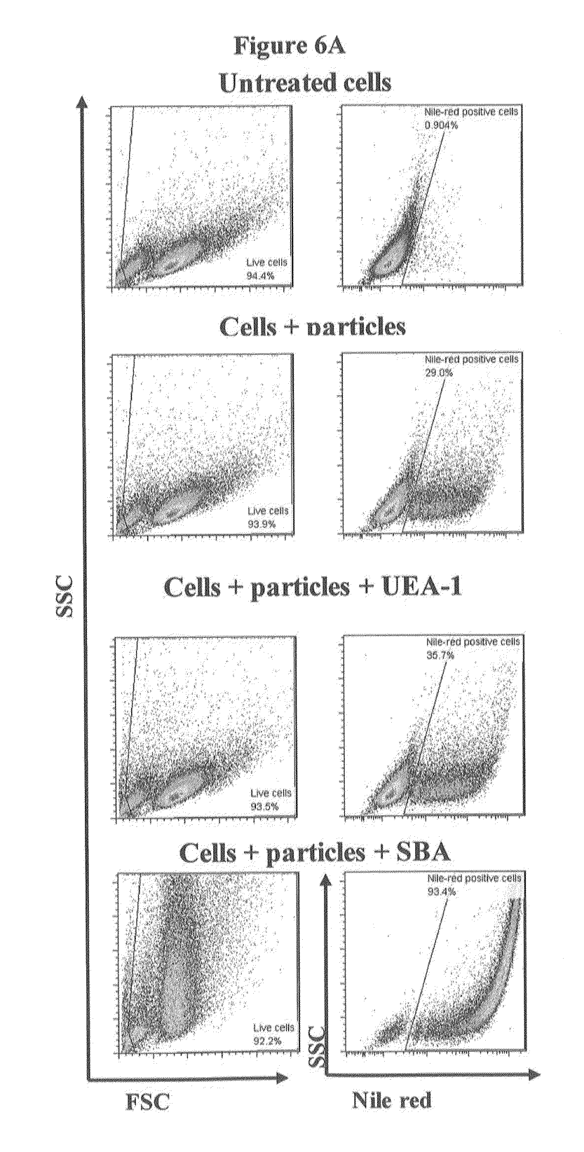

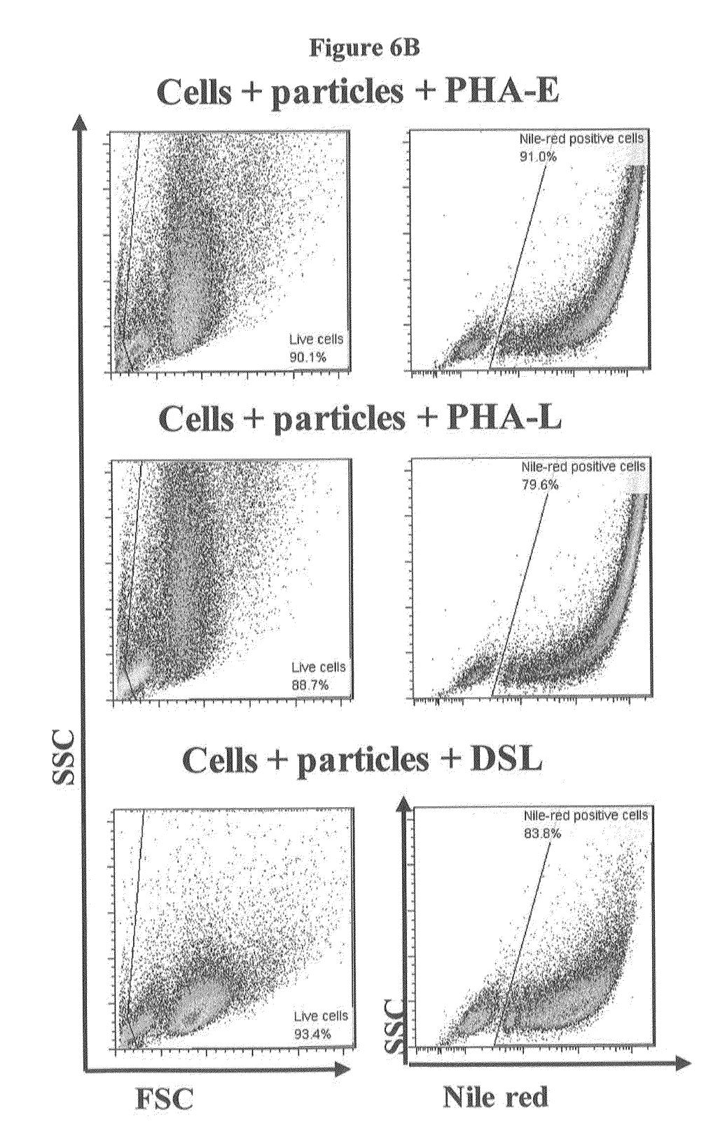

As shown in FIGS. 6A-6B, following a 10 minute incubation, 35.7% of the live iBMMs were found to have taken up NR-PS particles conjugated with biotinylated UEA-1 (100 mg/ml), 93.4% of the live iBMMs were found to have taken up NR-PS particles conjugated with biotinylated SBA (100 μg/ml), 91.0% of the live iBMMs were found to have taken up NR-PS particles conjugated with biotinylated PHA-E (100 μg/ml), 79.6% of the live iBMMs were found to have taken up NR-PS particles conjugated with biotinylated PHA-L (100 μg/ml) and 83.8% of the live iBMMs were found to have taken up NR-PS particles conjugated with biotinylated DSL (100 μg/ml), whereas only 29.0% of the live iBMMs were found to have taken up unconjugated NR-PS particles under similar conditions.

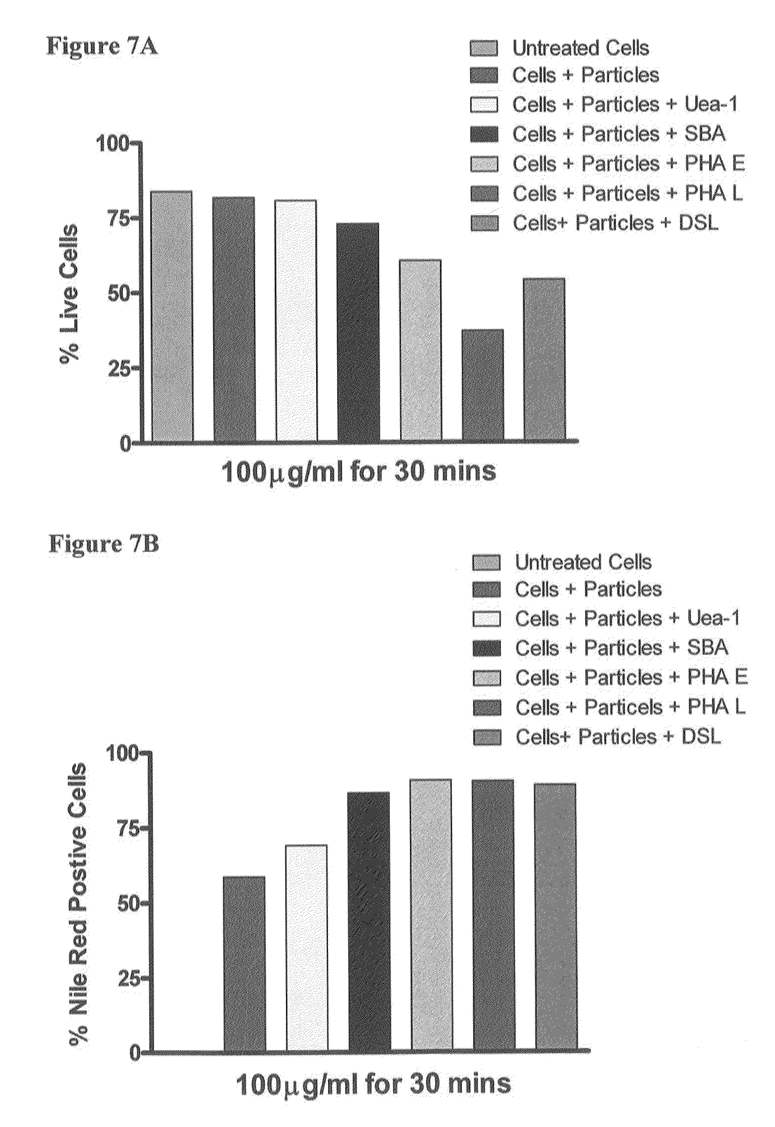

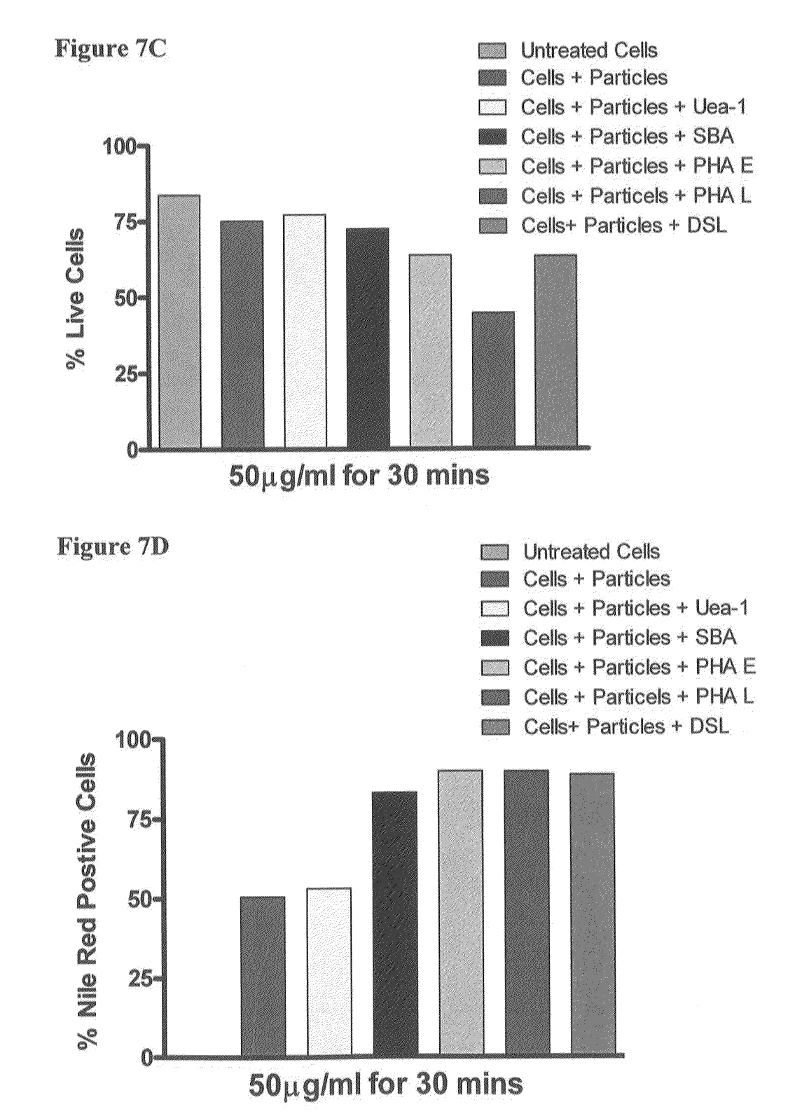

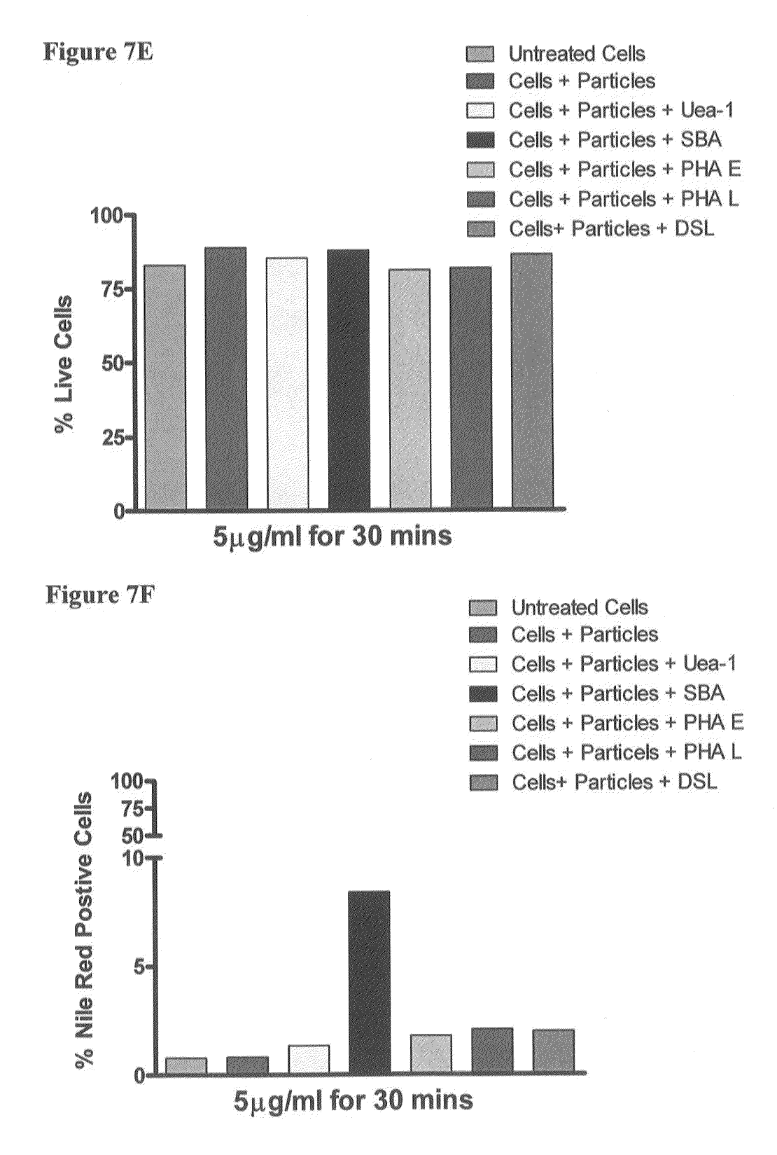

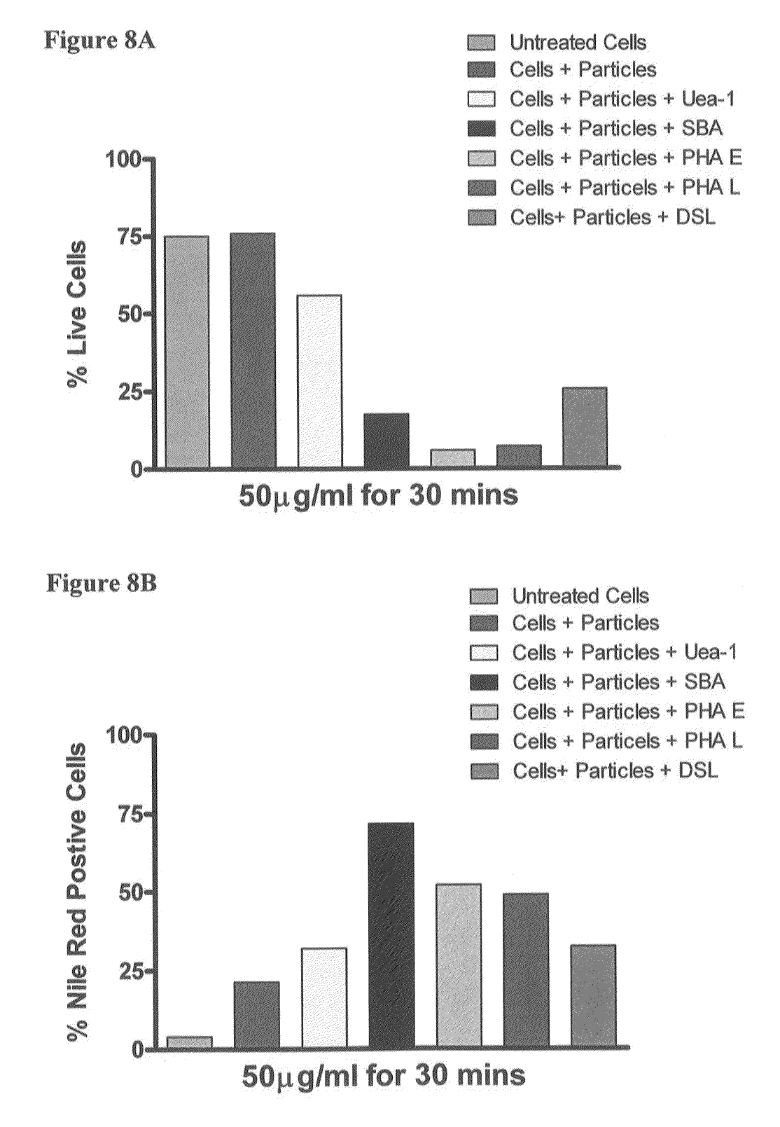

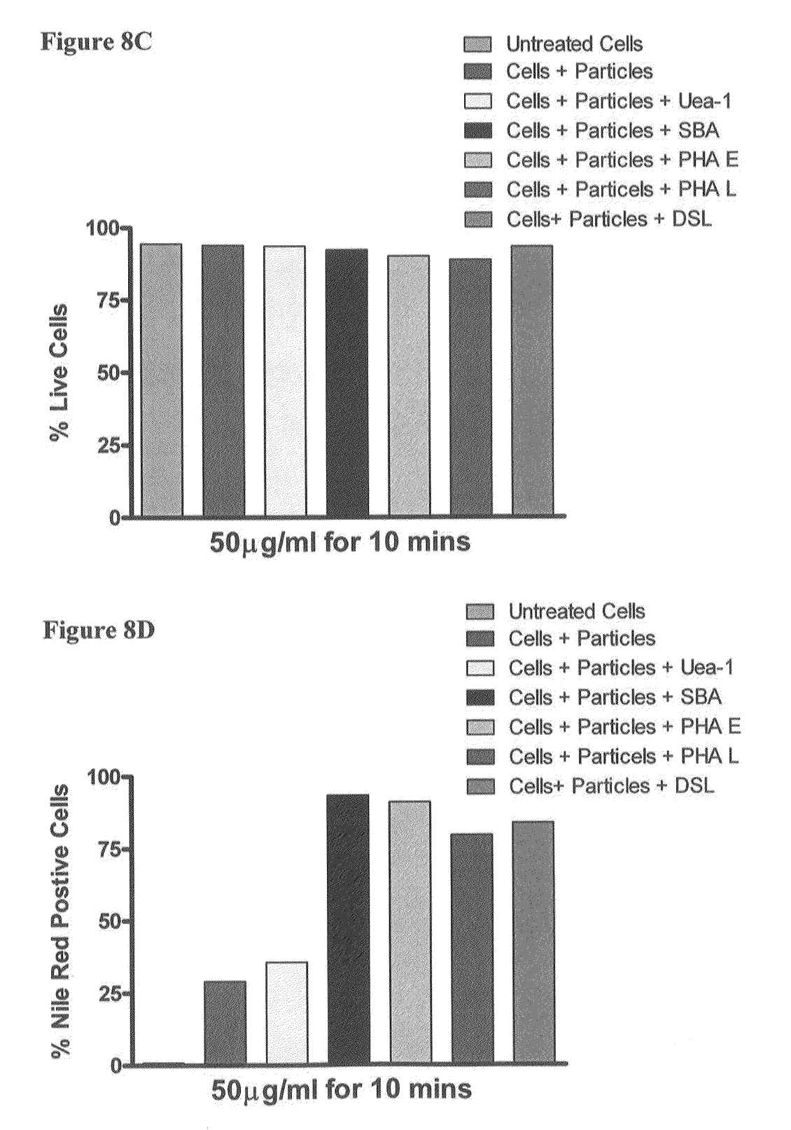

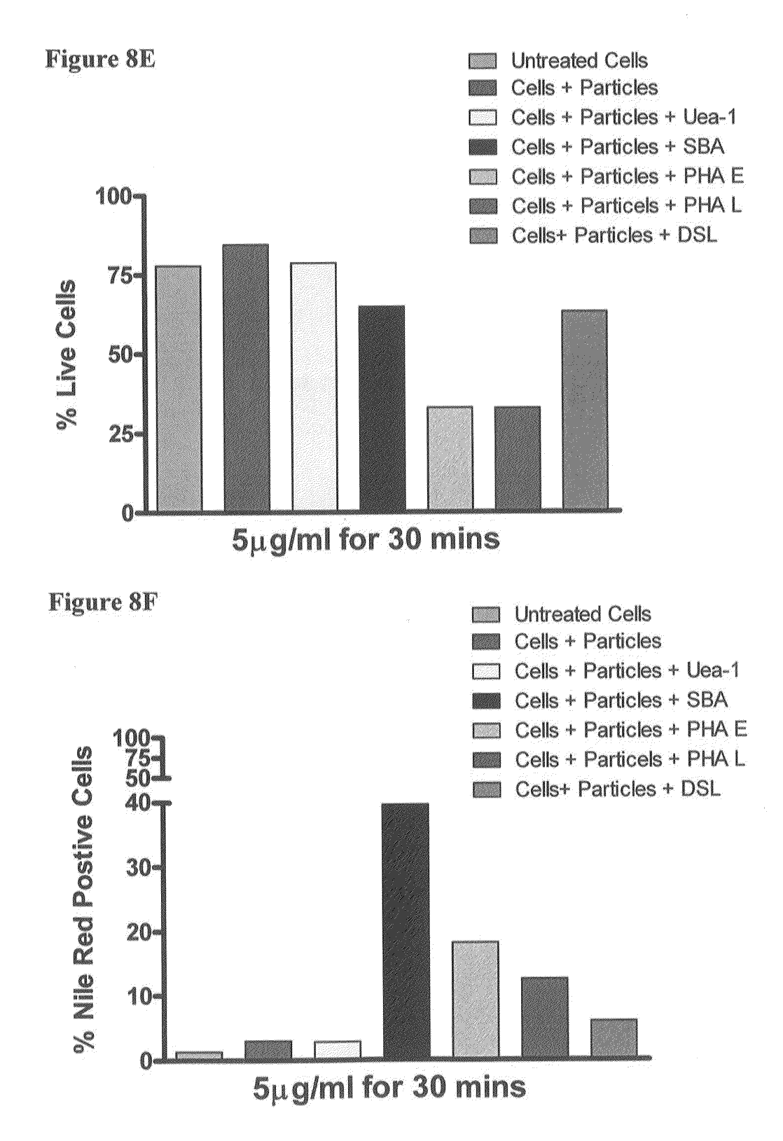

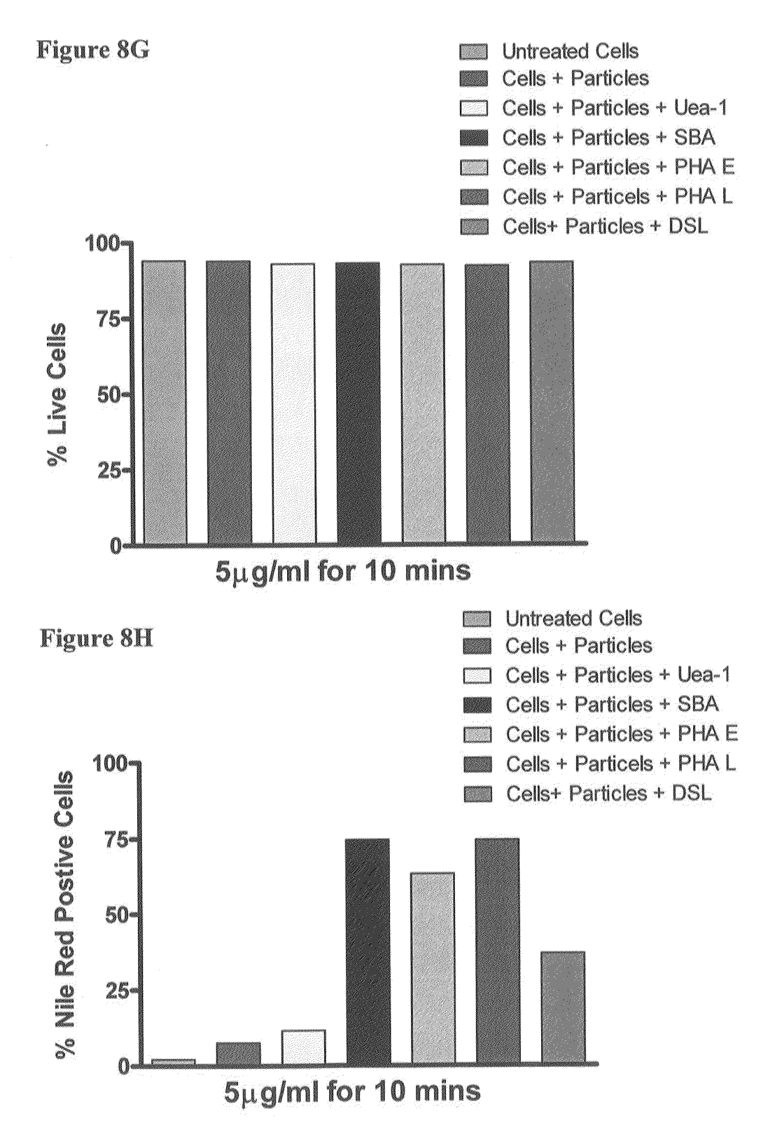

As shown in FIGS. 7B, 7D, 7F and 8B, 8D, 8F, 8H, the lectins effectively targeted the NR-PS particles to BMDCs and iBMMS at various concentrations and incubation periods. Cell viability was altered somewhat by the lectin-targeted particles, and was lectin-, time- and particle concentration-dependent (FIGS. 7A, 7C, 7E and 8A, 8C, 8E, 8G).

Thus, conjugating biotinylated lectins to particles appears to target the particles to both dendritic cells and macrophages.

### Example 7

### UEA-1 Targeting Increases Particle Uptake by Multiple Leukocyte Types

C3H/HeJ splenocytes were isolated from mice and cultured as described above, at a density of 1×10⁶ cells/ml in a 96 well U-bottomed plate, in 100 μl of complete RPMI 1640 medium. Cells were stimulated for 2 hours at 37° C. with NR-PS particles (1.0 mg/ml) or NR-PS particles (1.0 mg/ml) conjugated with biotinylated UEA-1 (100 μg/ml). Cells were then scraped into FACS tubes, washed in FACS buffer, centrifuged at 1200 rpm for 5 minutes (×3) and resuspended in 100 μl FACS buffer. Cells were then incubated with Fc Block™ (2.5 μg/ml; BD Pharmingen, San Diego, Calif.) for 10 minutes. Determination of cell types was achieved by staining with fluorescently-labelled antibodies specific for characteristic cell surface markers—monocytes were determined as being CD11b⁺/CD14⁺, granulocytes Gr1⁺/CD11b⁺, dendritic cells CD11c⁺, B cells CD19⁺ and T cells CD3⁺. Cells were incubated on ice for 30 minutes in the dark and then washed in FACS buffer and centrifuged at 1200 rpm for 5 minutes (×3). After washing, cells were resuspended in 200 μl of FACS buffer.

A CyAn™ ADP flow cytometer (Beckman Coulter, Inc., Miami, Fla.), Summit™ software (Dako North America, Inc., Carpinteria, Calif.) and FlowJo™ software (Treestar, Inc., Ashland, Oreg.) were used to analyze the uptake of particles by various cells populations. Particle uptake was quantified by determining the percentage of cells taking up particles and by determining the mean fluorescence intensity (MFI), which represents the number of particles taken up per cell. Unstimulated cells were used as controls.

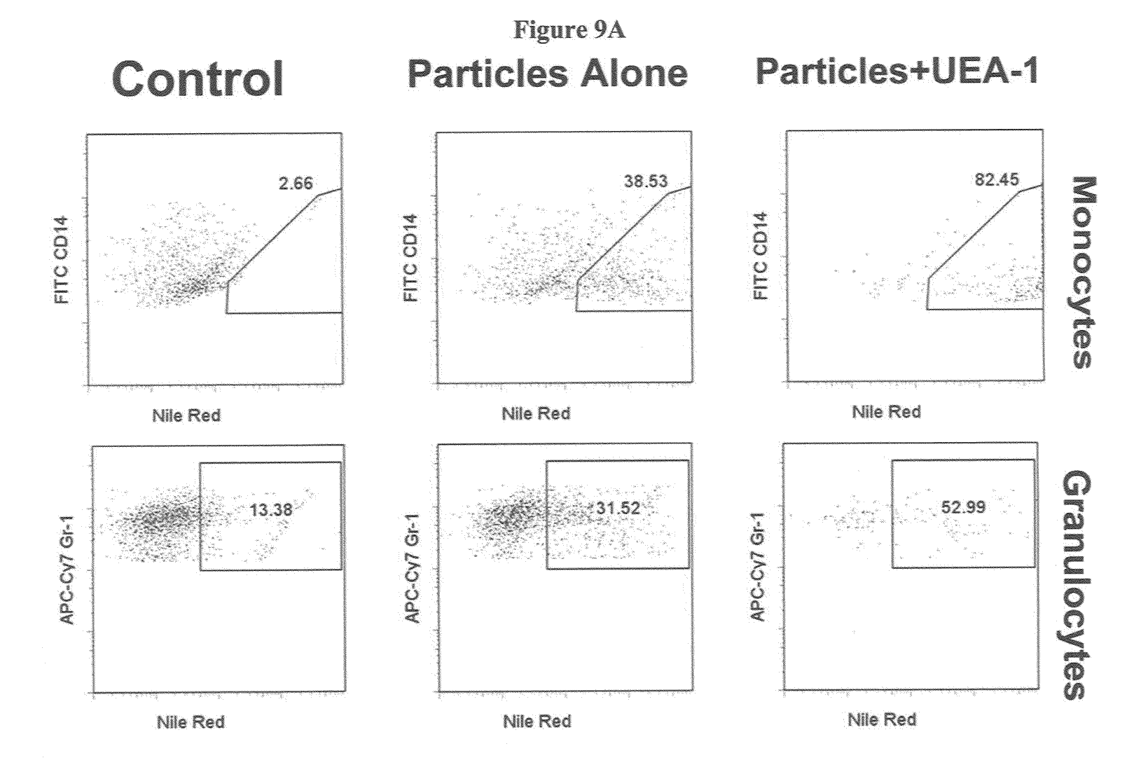

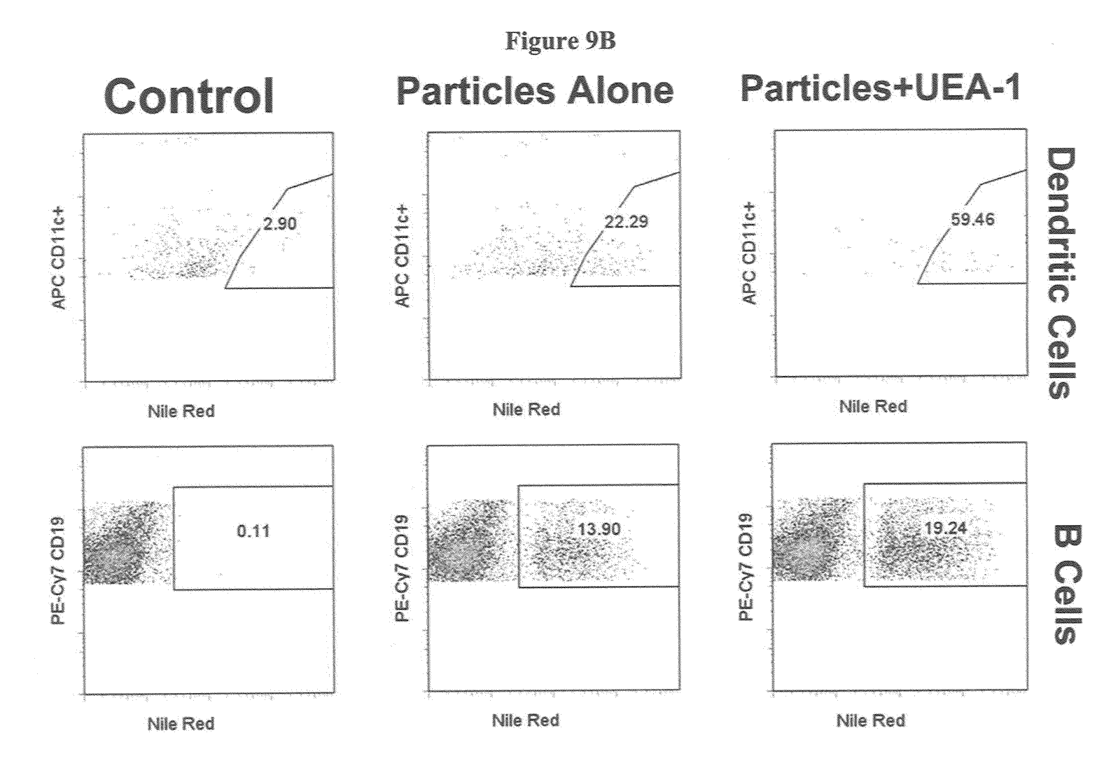

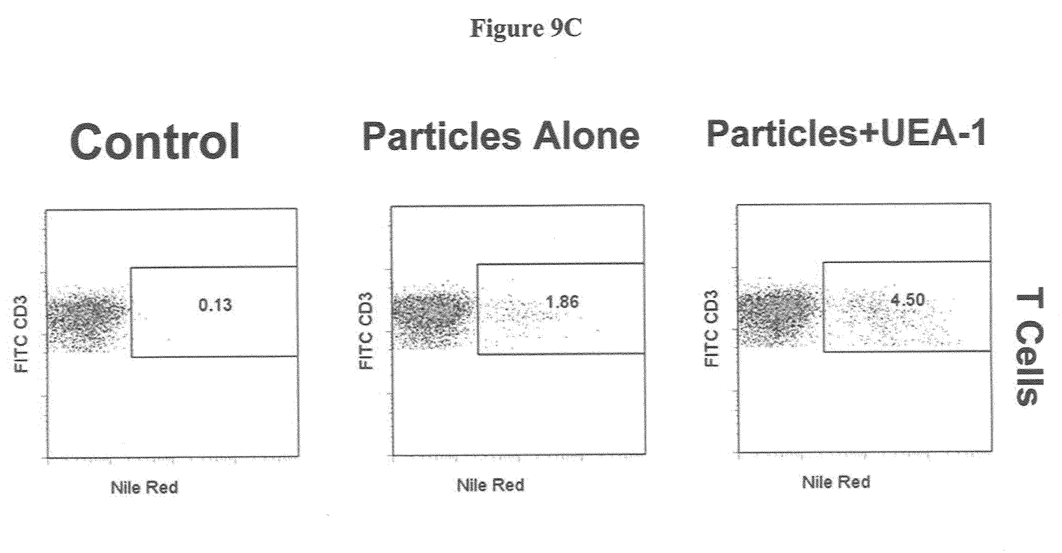

As shown in FIGS. 9A-9C, UEA-1 appears to target multiple leukocyte types. 82.45% of the monocytes were found to have taken up NR-PS particles conjugated with biotinylated UEA-1, whereas only 38.53% of the monocytes were found to have taken up unconjugated NR-PS particles. 52.99% of the granulocytes were found to have taken up NR-PS particles conjugated with biotinylated UEA-1, whereas only 31.52% of the granulocytes were found to have taken up unconjugated NR-PS particles. 59.46% of the dendritic cells were found to have taken up NR-PS particles conjugated with biotinylated UEA-1, whereas only 22.29% of the dendritic cells were found to have taken up unconjugated NR-PS particles. 19.34% of the B cells were found to have taken up NR-PS particles conjugated with biotinylated UEA-1, whereas only 13.90% of the B cells were found to have taken up unconjugated NR-PS particles. 4.50% of the T cells were found to have taken up NR-PS particles conjugated with biotinylated UEA-1, whereas only 1.86% of the T cells were found to have taken up unconjugated NR-PS particles. In other words, conjugation with biotinylated UEA-1 increased the percentage of cells taking up particles by 113.99% amongst monocytes, 68.11% amongst granulocytes, 166.76% amongst dendritic cells, 38.42% amongst B cells and 141.94% amongst T cells.

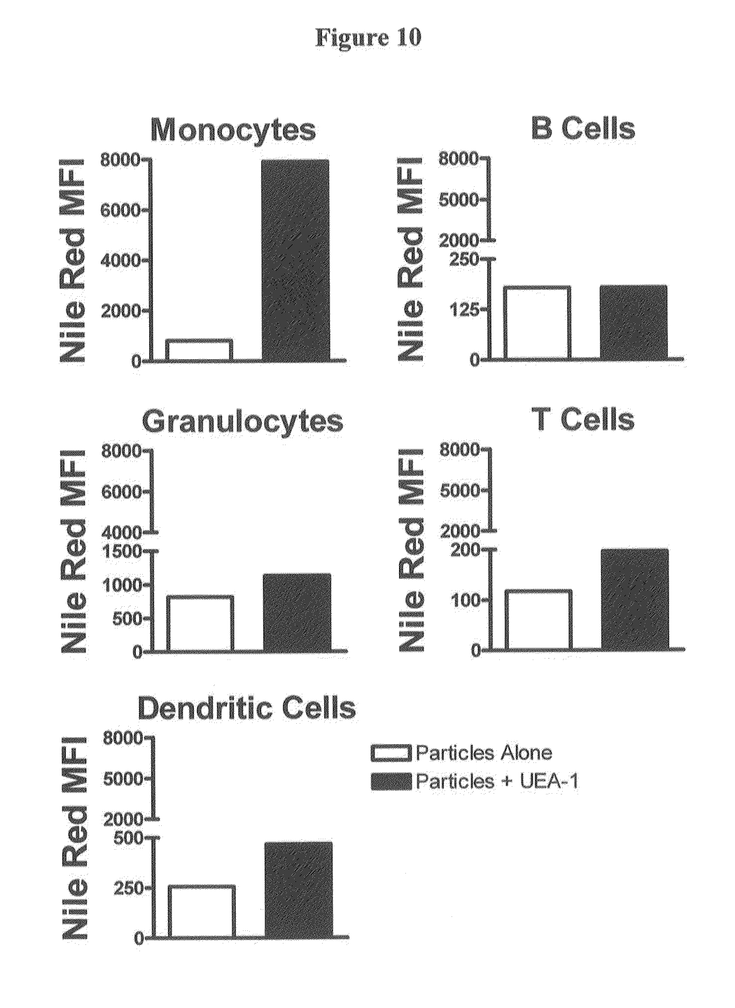

As shown in FIG. 10, conjugation with biotinylated UEA-1 increased the number of particles taken up per cell by monocytes (7,896 vs. 814), granulocytes (1,134 vs. 816), dendritic cells (467 vs. 257) and T cells (192 vs. 117), as determined by MFI values, but led to no enhancement of MFI in B cells.

Thus, UEA-1 appears to target multiple leukocytes, including monocytes, granulocytes and dendritic cells.

### Example 8

### Lectin Targeting Increases Particle Uptake by Multiple Leukocyte Types

Splenocytes were isolated from C57BL/6 mice and cultured as described above, at a density of 2×10⁶ cells/ml in a 96 well U-bottomed plate, in 100 μl of complete RPMI 1640 medium. Cells were incubated for 5, 10 or 30 minutes at 37° C. with NR-PS particles (5, 50 or 100 μg/ml) or NR-PS particles (5, 50 or 100 μg/ml) conjugated with biotinylated UEA-1 (100 μg/ml), biotinylated SBA (100 μg/ml), biotinylated PHA-E (100 μl/ml), biotinylated PHA-L (100 μg/ml) or biotinylated DSL (100 μg/ml). Cells were transferred to FACS tubes, washed in FACS buffer, centrifuged at 1200 rpm for 5 minutes (×3) and resuspended in 100 μl FACS buffer. Cells were then incubated with Fc Block™ (2.5 μg/ml; BD Pharmingen, San Diego, Calif.) for 10 minutes. Determination of cell types was achieved by labelling characteristic cell surface markers with fluorescently-labelled antibodies—T cells were determined as being CD3⁺, dendritic cells CD11c⁺, macrophages F4/80⁺, granulocytes Gr1⁺ and B cells CD19⁺. Cells were incubated on ice for 30 minutes in the dark and then washed in FACS buffer and centrifuged at 1200 rpm for 5 minutes (×3). After washing, cells were resuspended in 200 μl of FACS buffer.

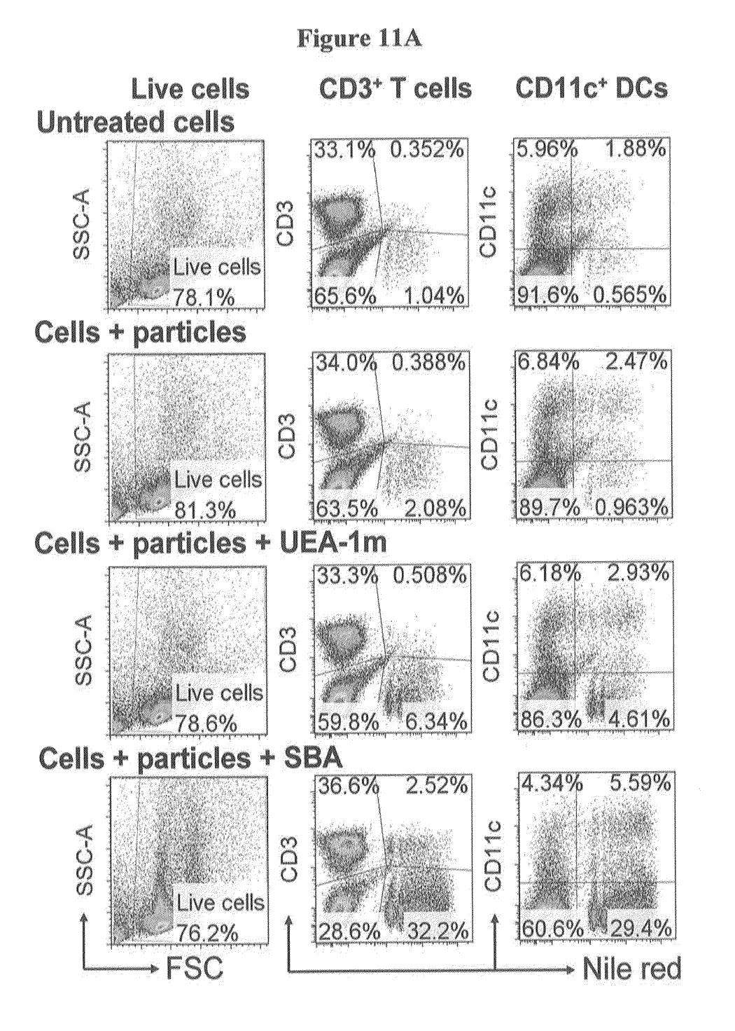

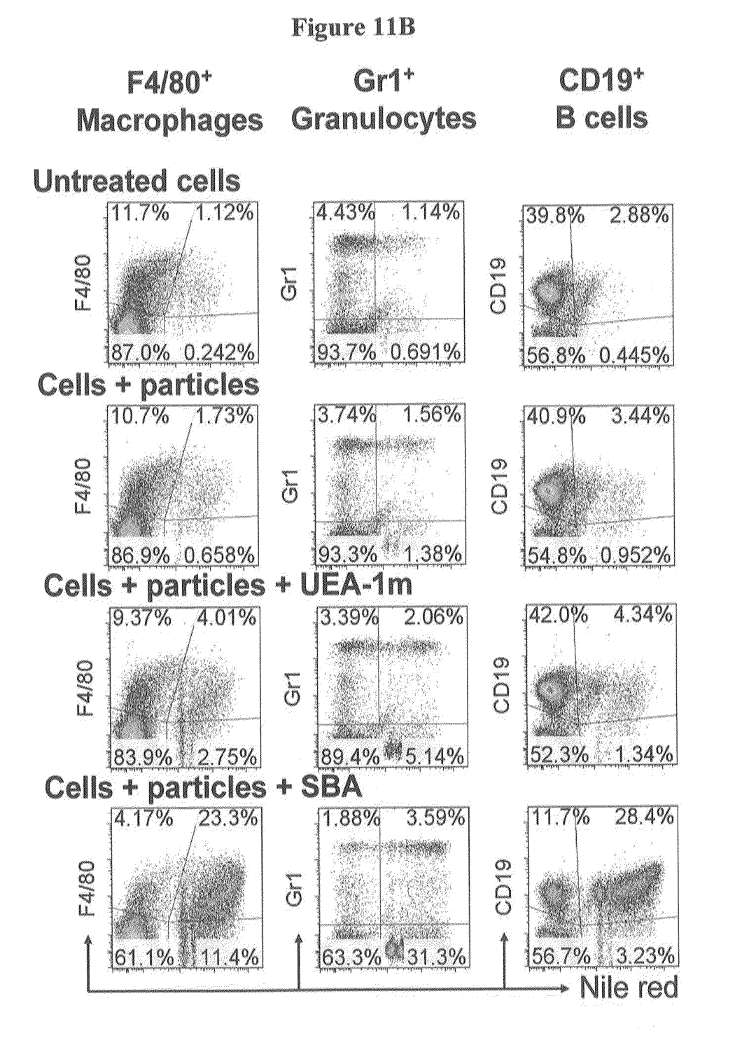

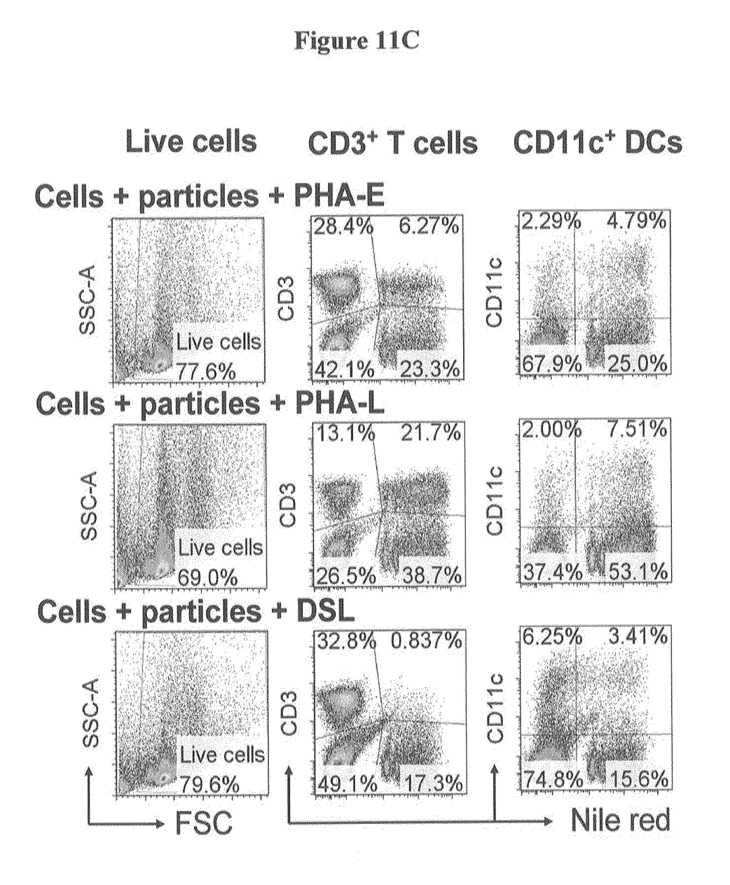

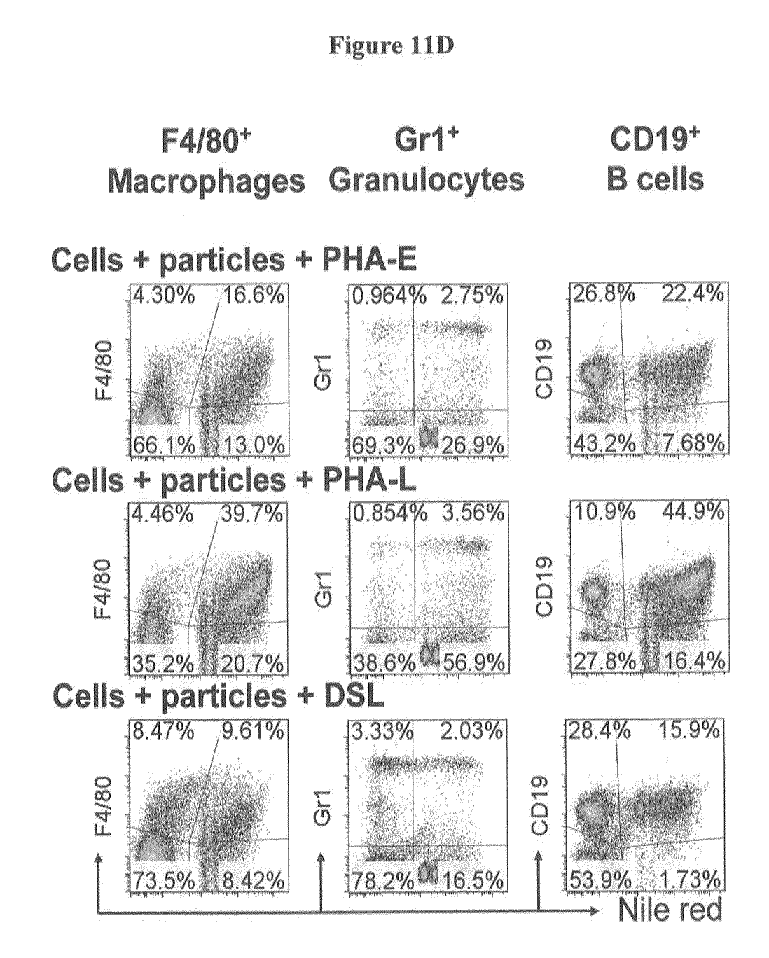

A CyAn™ ADP flow cytometer (Beckman Coulter, Inc., Miami, Fla.), Summit™ software (Dako North America, Inc., Carpinteria, Calif.) and FlowJo™ software (Treestar, Inc., Ashland, Oreg.) were used to analyze the uptake of particles by various cells populations. Live cells were gated on by their FSC and SSC properties in order to roughly estimate the degree of cell death. Particle uptake was calculated for each cell subtype from the data in FIGS. 11A-11D, showing the percentages of both cell marker- and particle-positive cells within the live cell population. Unstimulated cells were used as controls.

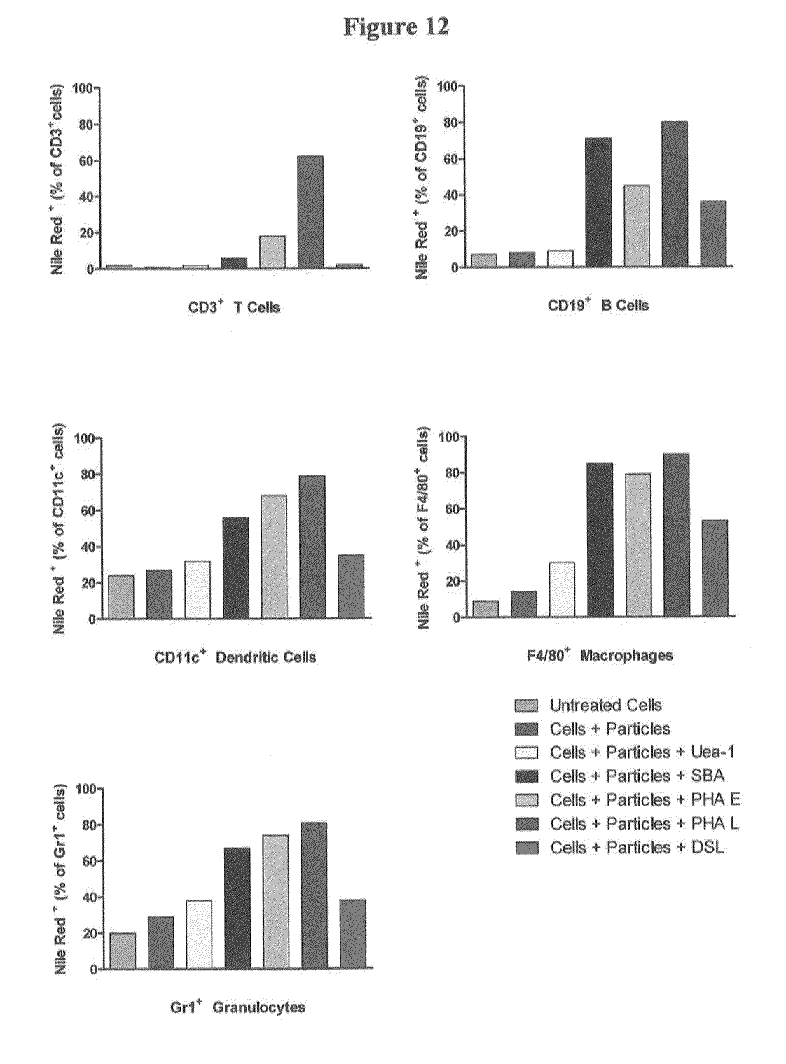

As shown in FIGS. 11A-11D and 12, the lectins appear to target multiple leukocyte types.

Whereas only 1.1% of the T cells were found to have taken up unconjugated NR-PS particles, 1.5% of the T cells were found to have taken up NR-PS particles conjugated with biotinylated UEA-1 (100 μg/ml), 6.4% of the T cells were found to have taken up NR-PS particles conjugated with biotinylated SBA (100 μg/ml), 18.1% of the T cells were found to have taken up NR-PS particles conjugated with biotinylated PHA-E (100 μg/ml), 62.4% of the T cells were found to have taken up NR-PS particles conjugated with biotinylated PHA-L (100 μg/ml) and 2.5% of the T cells were found to have taken up NR-PS particles conjugated with biotinylated DSL (100 μg/ml).

Whereas only 26.5% of the dendritic cells were found to have taken up unconjugated NR-PS particles, 32.2% of the dendritic cells were found to have taken up NR-PS particles conjugated with biotinylated UEA-1 (100 μg/ml), 56.3% of the dendritic cells were found to have taken up NR-PS particles conjugated with biotinylated SBA (100 μg/ml), 67.7% of the dendritic cells were found to have taken up NR-PS particles conjugated with biotinylated PHA-E (100 μg/ml), 79.0% of the dendritic cells were found to have taken up NR-PS particles conjugated with biotinylated PHA-L (100 μg/ml) and 35.3% of the dendritic cells were found to have taken up NR-PS particles conjugated with biotinylated DSL (100 μg/ml).

Whereas only 13.9% of the macrophages were found to have taken up unconjugated NR-PS particles, 30.0% of the macrophages were found to have taken up NR-PS particles conjugated with biotinylated UEA-1 (100 μg/ml), 84.8% of the macrophages were found to have taken up NR-PS particles conjugated with biotinylated SBA (100 μg/ml), 79.4% of the macrophages were found to have taken up NR-PS particles conjugated with biotinylated PHA-E (100 μg/ml), 89.9% of the macrophages were found to have taken up NR-PS particles conjugated with biotinylated PHA-L (100 μg/ml) and 53.2% of the macrophages were found to have taken up NR-PS particles conjugated with biotinylated DSL (100 μg/ml).

Whereas only 29.4% of the granulocytes were found to have taken up unconjugated NR-PS particles, 37.8% of the granulocytes were found to have taken up NR-PS particles conjugated with biotinylated UEA-1 (100 μg/ml), 65.6% of the granulocytes were found to have taken up NR-PS particles conjugated with biotinylated SBA (100 μg/ml), 74.0% of the granulocytes were found to have taken up NR-PS particles conjugated with biotinylated PHA-E (100 μg/ml), 80.7% of the granulocytes were found to have taken up NR-PS particles conjugated with biotinylated PHA-L (100 μg/ml) and 37.9% of the granulocytes were found to have taken up NR-PS particles conjugated with biotinylated DSL (100 μg/ml).

Whereas only 7.8% of the B cells were found to have taken up unconjugated NR-PS particles, 9.4% of the B cells were found to have taken up NR-PS particles conjugated with biotinylated UEA-1 (100 μg/ml), 70.8% of the B cells were found to have taken up NR-PS particles conjugated with biotinylated SBA (100 μg/ml), 45.5% of the B cells were found to have taken up NR-PS particles conjugated with biotinylated PHA-E (100 μg/ml), 80.5% of the B cells were found to have taken up NR-PS particles conjugated with biotinylated PHA-L (100 μg/ml) and 35.9% of the B cells were found to have taken up NR-PS particles conjugated with biotinylated DSL (100 μg/ml).

Thus, lectins appear to target multiple leukocytes, including T cells, dendritic cells, macrophages, granulocytes and B cells.

### Example 9

### Enzyme-Linked Immunosorbant Assay (ELISA)

The concentrations of cytokines secreted following stimulation with PS particle preparations were measured by ELISA.

Plate Reading

Absorbance was measured at a wavelength of 492 nm using an ELISA plate reader (Versa Max Microplate Reader). The resulting data was analyzed using SoftMax® Pro Data Acquisition & Analysis Software (Molecular Devices, Inc., Sunnyvale, Calif.). Unknown protein concentrations were determined by reading from a standard curve.

Cytokine Quantification by ELISA

BMDCs were isolated and cultured as described in Example 2 at a density of 6.25×10⁵ cells/ml in 96 well U-bottomed microplates. Cells were stimulated with a Toll-like receptor (TLR) ligand (LPS or Pam2CSK4) for 6 hours. Cells were then incubated with either medium, alum/SC-PS particles, alum/SC-PS particles conjugated with UEA-1, alum/SC-PS particles conjugated with UEA-1 mimetic or with UEA-1 alone for 24 hours. After incubation, supernatants from BMDCs were collected and cytokine concentrations measured by ELISA. Antibody pairs specific for each cytokine were used for immunoassaying. The following cytokines were measured by immunoassay: IL-1α, IL-1β.

Standard Cytokine ELISA Protocol

Capture antibodies were obtained from BD Pharmingen (San Diego, Calif.), BioLegend (San Diego, Calif.) and R&D Systems, Inc. (Minneapolis, Minn.) and prepared according to the manufacturer's specifications (see Table 2) and a volume of 40 μl/well added to high-binding 96 well ELISA plates. Plates were then incubated for 2 hours at 37° C. or overnight at 4° C. After incubation, plates were washed in PBS-T (×3) and tapped dry. Plates were then blocked with the appropriate blocking solution (see Table 2) and incubated for 2 hours at 37° C. After incubation plates were washed in PBS-T (×3) and tapped dry. Supernatants were transferred from cell culture plates to fresh 96 well plates. All supernatants were stored at −20° C. when not in use. Cell supernatants were applied to plates at the indicated dilutions (see Table 2). A blank triplicate was left on each plate containing the diluent as a blank. Standards were prepared at the starting concentration in the recommended diluent as specified by the manufacturer and transferred to a 96 well plate and serial dilutions (1:2) performed (see Table 2). All standards and samples were applied to plates at 40 μl/well total volume for incubation overnight at 4° C. After incubation plates were washed with PBS-T (×5) and tapped dry. Detection antibody was then diluted in the diluent as per manufacturer's instructions (see Table 2) and added to plates at 40 μl/well. The plates were left at room temperature at the indicated times in the dark (see Table 2) and washed in PBS-T (×3) and tapped dry. Streptavidin-HRP was diluted in the same diluent as the detection antibody and 40 μl/well added to the plate. This was allowed to incubate at room temperature for 20 minutes in the dark. Plates were once again washed in PBS-T (×3) and tapped dry before 40 μl/well of substrate solution was added. Plates were then stopped by the addition of 20 μl/well of 1M H₂SO₄ and read.

TABLE 2
ELISA Antibodies

| | | Capture | | | Top working | |
|---|---|---|---|---|---|---|
| | | Antibody | Blocking | Sample | standard Ab | Detection |
| Cytokine | Source | (in PBS) | Solution | Dilution | concentration | Antibody |
| IL-1α | Biolegend | 1:200 | 1% BSA | 1:2 in | 2000 pg/ml in | 1:200 |
| | | | | 0.1% PBS-T | 0.1% PBS-T | in 0.1% PBS-T |
| IL-1β | R&D | 1:180 | 1% BSA | 1:2 in | 2000 pg/ml in | 1:180 |
| | Systems | | | 1% BSA | 1% BSA | in 1% BSA |
| IL-5 | BD | 1:500 | 10% Milk | 1:2 in | 2500 pg/ml in | 1:500 |
| | Pharmingen | | | PBS | PBS | in PBS |
| IL-10 | R&D | 1:180 | 1% BSA | 1:2 in | 2000 pg/ml in | 1:180 |
| | Systems | | | 1% BSA | 1% BSA | in 1% BSA |
| IL-17 | R&D | 1:180 | 1% BSA | 1:2 in 1% BSA | 1000 pg/ml in | 1:180 |
| | Systems | | | | 1% BSA | in 1% BSA |
| IFN-γ | BD | 1:1000 | 10% Milk | 1:2 in | 4000 pg/ml in | 1:500 |
| | Pharmingen | | | PBS | PBS | in PBS |

### Example 10

### Targeting Particles to Dendritic Cells with UEA-1 Increases Particle-Driven IL-1α and IL-10 Cytokine Production

In order to determine whether the conjugation of biotinylated UEA-1 to SC-PS particles influenced TLR4-primed IL-1α and IL-1β cytokine production in vitro, the following experiment was undertaken.

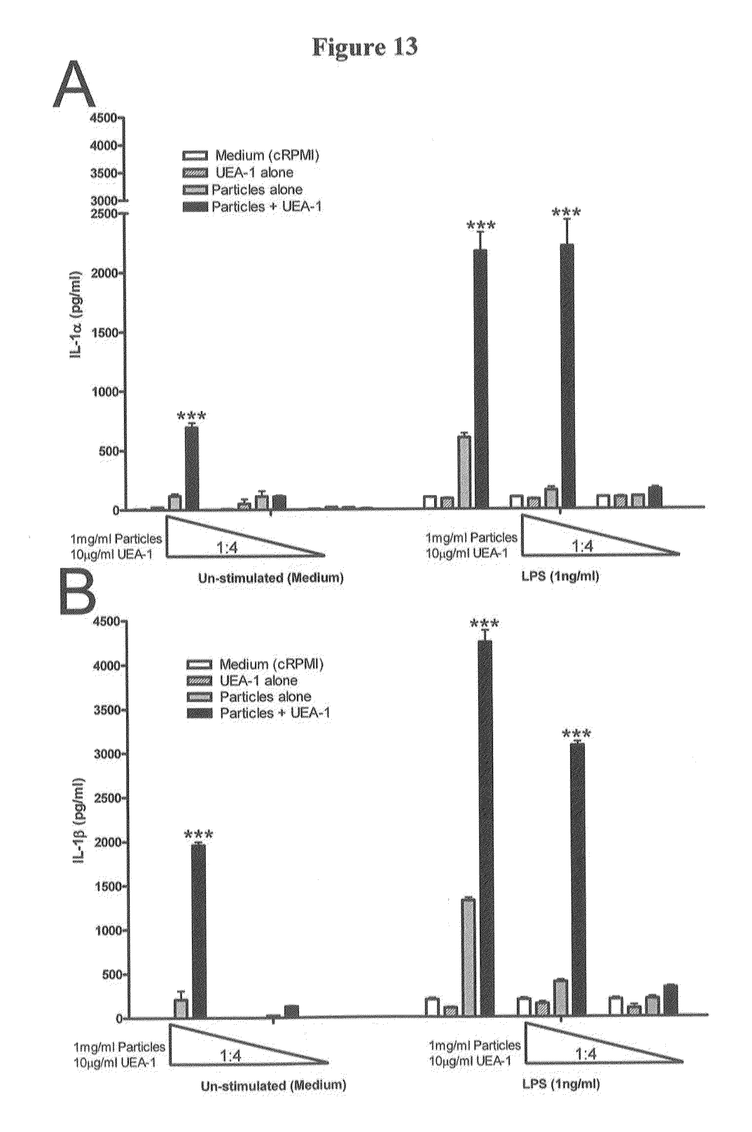

Murine C57BL/6 BMDCs (6.25×10⁵ cells/ml) were stimulated with LPS (1 ng/ml) for 6 hours or left unstimulated. After 6 hours, these cells were incubated with serially diluted SC-PS particles, serially diluted SC-PS particles conjugated with biotinylated UEA-1 or with biotinylated UEA-1 alone. After 24 hour incubation, supernatants were assayed for IL-1α (FIG. 13A) and IL-1β (FIG. 13B) by ELISA.

A significant enhancement (p<0.001) of IL-1α production was seen at the higher SC-PS particle concentrations (1 mg/ml) with conjugated UEA-1 (FIG. 13A) in dendritic cells absent from LPS stimulation compared to SC-PS particles alone. At lower particle concentrations no significant enhancement (p>0.05) of IL-1α production was observed (FIG. 13A).

A significant (p<0.001) increase in IL-1α production by dendritic cells stimulated with LPS was observed when biotinylated UEA-1 was conjugated to SC-PS particles, compared to SC-PS particles alone (FIG. 13A) at particle concentrations ranging from 1 mg/ml to 250 μg/ml. At lower particle concentrations, no significant increase in IL-1α production was seen when biotinylated UEA-1 was conjugated to SC-PS particles. Some IL-1α was produced by dendritic cells on their own.

In the absence of LPS stimulation, there was a significant (p<0.001) increase in IL-1β production by dendritic cells stimulated with targeted SC-PS particles (1 mg/ml) compared to untargeted SC-PS particles (FIG. 13B). Particle concentrations below 0.25 mg/ml did not induce IL-1β production even when targeted with UEA-1 (FIG. 13B).

When dendritic cells were stimulated with LPS, cells produced a small amount of IL-1β on their own. UEA-1 targeting of SC-PS particles induced a significant increase (p<0.001) in IL-1β production by dendritic cells at SC-PS particle concentrations from 1 mg/ml to 250 μg/ml (FIG. 13B). At a SC-PS particle concentration of 62.5 μg/ml the increase in IL-1β by attaching biotinylated UEA-1 was not significant (p>0.5) (FIG. 13B). A small amount of IL-1β was also produced by dendritic cells incubated with biotinylated UEA-1 on its own (FIG. 13B). It thus appears that targeting SC-PS particles to dendritic cells with UEA-1 induces a very strong enhancement of IL-1α and IL-1β production by these cells in vitro.

### Example 11

### Adsorption of UEA-1 onto Particles Enhances IL-1α and IL-1β Production by Dendritic Cells

Next, an experiment was undertaken to determine if the method by which UEA-1 is attached to the particles has an effect on the TLR4-primed production of IL-1α and IL-1β by dendritic cells in vitro.

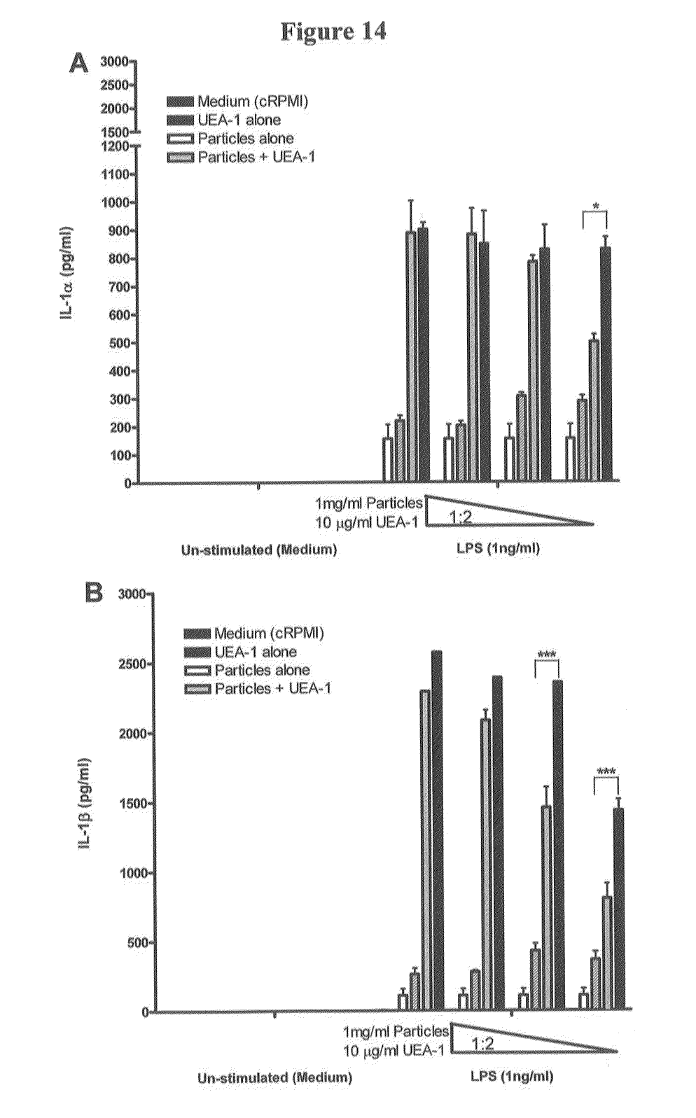

Murine C3H/HeN BMDCs (6.25×10⁵ cells/ml) were stimulated with LPS (1 ng/ml) for 6 hours or left unstimulated. After 6 hours, these cells were incubated with serially diluted PS particles, serially diluted PS particles with adsorbed UEA-1 or UEA-1 alone for 24 hours. After 24 hour incubation, supernatants were assayed for IL-1α (FIG. 14A) and IL-1β (FIG. 14B) by ELISA.

No IL-1α (FIG. 14A) or IL-1β (FIG. 14B) was produced by any dendritic cells in the absence of LPS stimulation when incubated with PS particles.

In LPS-stimulated dendritic cells, UEA-1-targeted PS particles only significantly increased IL-1α production at the 0.125 mg/ml PS particle concentration alone (p<0.05). At all other concentrations there was no enhancement of IL-1α production (p>0.05) (FIG. 14A). IL-1β production by LPS-stimulated dendritic cells was significantly increased (p<0.001) at the two lowest concentrations of PS particles (0.25 mg/ml and 0.125 mg/ml) when targeted with UEA-1 (FIG. 14B). No significant enhancement (p>0.05) of IL-1β production by dendritic cells was observed at the higher PS particle amounts when targeted with UEA-1. Thus, attachment of UEA-1 by adsorption to PS particles appears to significantly enhance TLR4-primed IL-1α and IL-β production in dendritic cells only at low concentrations of particles in vitro.

### Example 12

### UEA-1 Does not Significantly Enhance Alum-Mediated Increases in IL-1α and IL-1β Cytokine Production by Dendritic Cells

Having shown that targeting with UEA-1 can enhance the ability of PS particles to promote the production of IL-1α and IL-1β by dendritic cells, it was next determined whether UEA-1 could also enhance the ability of alum to promote the production of IL-1α and IL-1β by dendritic cells.

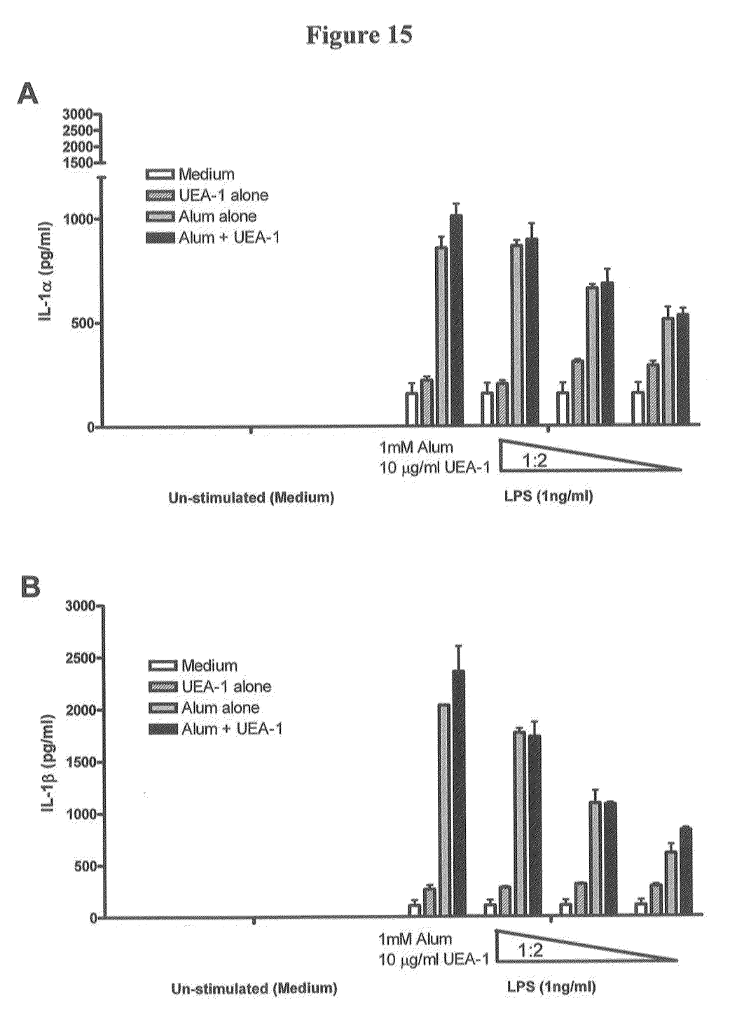

Murine C3H/HeN BMDCs (6.25×10⁵ cells/ml) were stimulated with LPS (1 ng/ml) for 6 hours or left unstimulated. After 6 hours, these cells were incubated with serially diluted alum alone, serially diluted alum with UEA-1 or UEA-1 alone. After 24 hour incubation, supernatants were assayed for IL-1α (FIG. 15A) and IL-1β (FIG. 15B) by ELISA.

There was no IL-1α (FIG. 15A) or IL-1β (FIG. 15B) production by dendritic cells in the absence of LPS stimulation.

In LPS-primed dendritic cells targeted with UEA-1, alum induced no significant (p>0.05) increase in IL-1α (FIG. 15A) or IL-1β (FIG. 15B) production compared to alum alone. Thus, UEA-1 does not appear to significantly enhance IL-1α or IL-1β production by dendritic cells stimulated with alum in vitro.

### Example 13

### Targeting Particles to Dendritic Cells with UEA-1 Mimetic Increases Particle-Driven IL-1α and IL-1β Cytokine Production

In order to determine if conjugation of UEA-1 mimetic to PS particles could enhance IL-1α and IL-1β production by dendritic cells, a UEA-1 mimetic developed by PolyPeptide Laboratories (San Diego, Calif.) was tested.

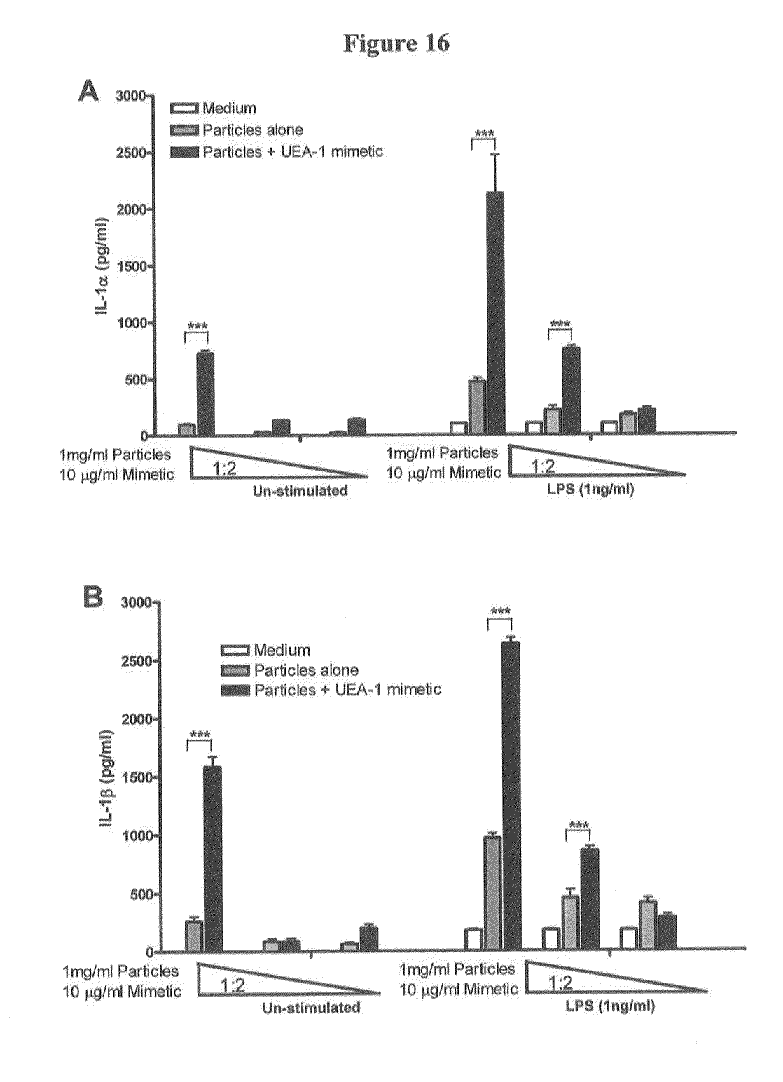

Murine C57BL/6 BMDCs (6.25×10⁵ cells/ml) were stimulated with LPS (1 ng/ml) for 6 hours or left unstimulated. After 6 hours, these cells were incubated with serially diluted SC-PS particles or serially diluted SC-PS particles conjugated with UEA-1 mimetic. It was not possible to investigate the effect of the UEA-1 mimetic alone because the concentration of DMSO used to solubilize the mimetic would prove toxic to the cells. After the 24 hour incubation, supernatants were assayed for IL-1α (FIG. 16A) and IL-1β (FIG. 16B) by ELISA.

In the absence of LPS stimulation, targeting of PS particles to dendritic cells with UEA-1 mimetic induced a significant increase (p<0.001) in both IL-1α (FIG. 16A) and IL-1β (FIG. 16B) production at a PS particle concentration of 1 mg/ml. At lower PS particle concentrations, targeting with UEA-1 mimetic did not induce a significant (p>0.05) enhancement of either IL-1α (FIG. 16A) or IL-1β (3 (FIG. 16B). When stimulated with LPS, PS particles targeted with UEA-1 mimetic significantly (p<0.001) increased IL-1α (FIG. 16A) and IL-1β (FIG. 16B) production by dendritic cells at 1 mg/ml and 0.5 mg/ml PS particle concentrations. At lower PS particle concentrations, no significant (p>0.05) enhancement of IL-1α (FIG. 16A) or IL-1β (FIG. 16B) was observed. It thus appears that targeting PS particles to dendritic cells with a UEA-1 mimetic significantly enhances IL-1α and IL-1β production.

### Example 14

### Particles Conjugated with UEA-1 Enhance IL-1α and IL-1β Production by Dendritic Cells to a Greater Extent than Particles Conjugated with a UEA-1 Mimetic

Having shown that both UEA-1 and a UEA-1 mimetic enhance IL-1α and IL-1β production by dendritic cells, the efficacy at which they do so was compared.

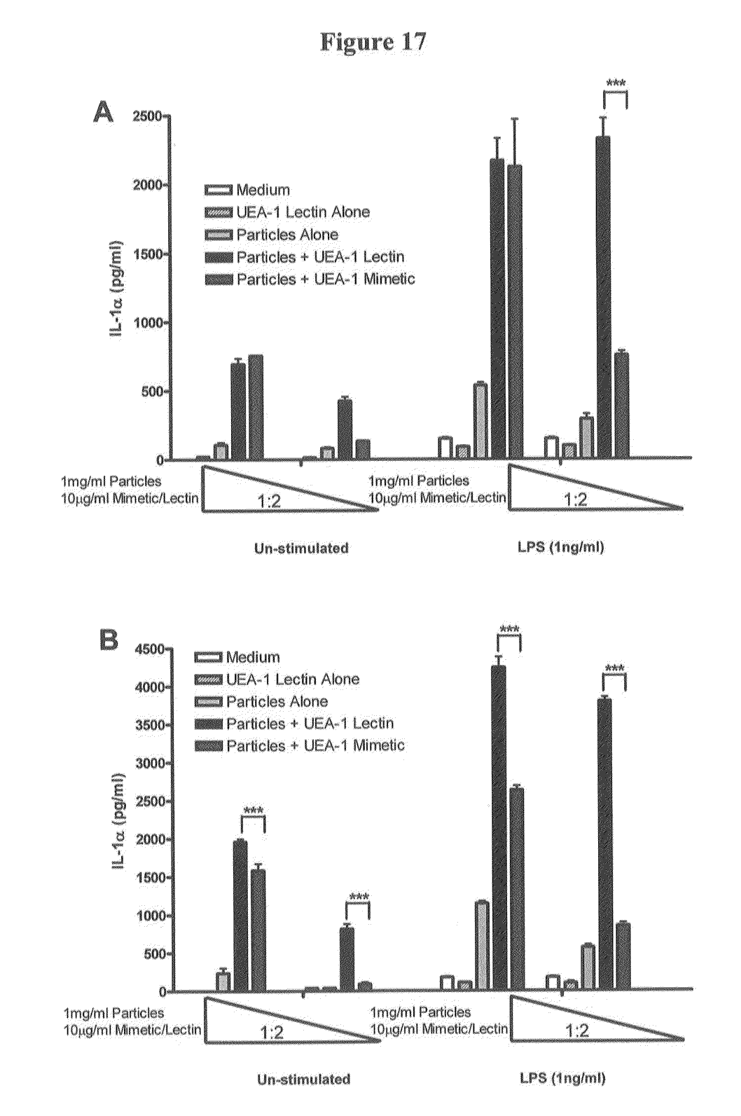

Murine C57BL/6 BMDCs (6.25×10⁵ cells/ml) were stimulated with LPS (1 ng/ml) for 6 hours or left unstimulated. After 6 hours, these cells were incubated with serially diluted SC-PS particles, serially diluted SC-PS particles conjugated with UEA-1 mimetic, serially diluted SC-PS particles conjugated with UEA-1, or UEA-1 alone. It was not possible to investigate the effect of the UEA-1 mimetic alone because the concentration of DMSO used to solubilize the mimetic would prove toxic to the cells. After the 24 hour incubation, supernatants were assayed for IL-1α (FIG. 17A) and IL-1β (3 (FIG. 17B) by ELISA.

In the absence of LPS stimulation, attachment of both the lectin and the mimetic to PS particles induced a similar enhancement of IL-1α at a particle concentration of 1 mg/ml. At 0.5 mg/ml targeting PS particles with UEA-1 increased IL-1α production over that of the mimetic (FIG. 17A).

When stimulated with LPS, targeting with UEA-1 induced a significant increase (p<0.001) in PS particle-mediated IL-1α production by dendritic cells over that induced by targeting with the mimetic at a PS particle concentration of 0.5 mg/ml (FIG. 17A). Without LPS stimulation, PS particle-mediated IL-β production by dendritic cells was significantly increased (p<0.001) when targeted with UEA-1 compared to the mimetic (FIG. 17B).

Dendritic cells stimulated with LPS produced significantly (p<0.001) more IL-1β at all PS particle concentrations when UEA-1 was used as a target molecule instead of the mimetic (FIG. 17B).

Thus, it appears that UEA-1-targeted particles induce a significantly greater enhancement of IL-1α and IL-β production by dendritic cells than their UEA-1 mimetic-targeted counterparts.

### Example 15

### The Enhancement of TLR-Activated IL-1α and IL-1β Production by Dendritic Cells in Response to UEA-1-Targeted Particles is not TLR-4-Specific

In order to determine whether the increase in IL-1α and IL-β production achieved by UEA-1 targeting of PS particles is specific for dendritic cells primed with TLR-4 agonists, the following experiment was carried out.

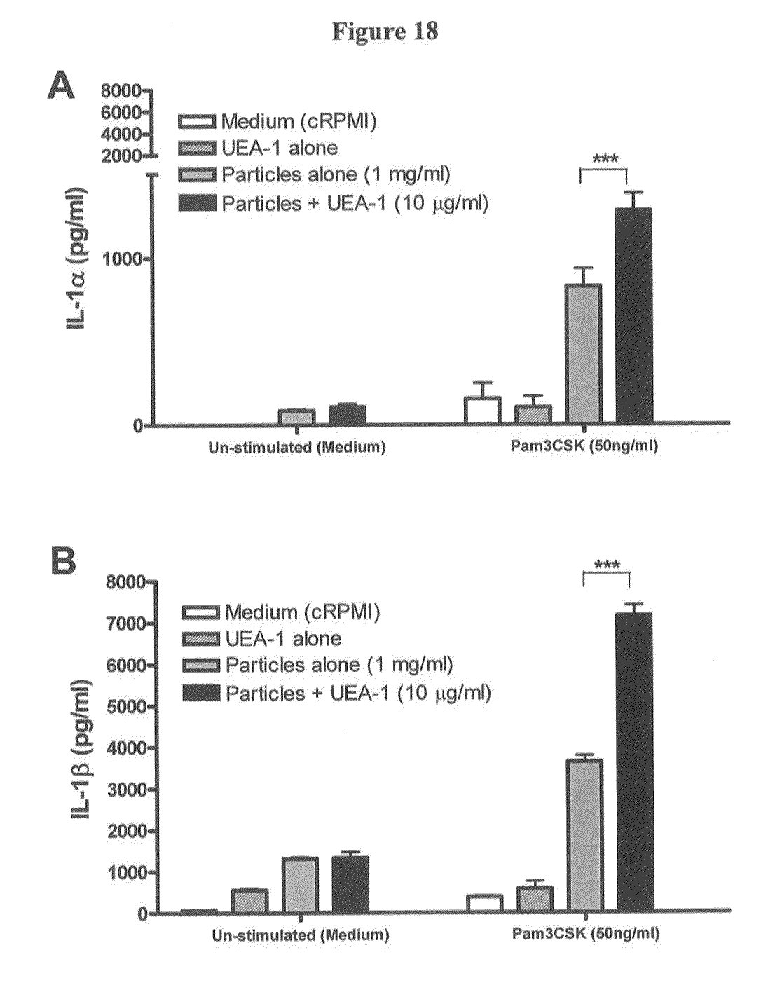

Murine C3H/HeJ BMDCs (6.25×10⁵ cells/ml) were stimulated with Pam3CSK (50 ng/ml) for 6 hours or left unstimulated. C3H/HeJ mice are not sensitive to LPS due to defective TLR-4 signalling, but are sensitive to other TLR agonists such as the TLR1/2 agonist, Pam3CSK. After 6 hours, unstimulated or PAM3CSK-stimulated cells were incubated with SC-PS particles (1 mg/ml), SC-PS particles conjugated with UEA-1 (10 μg/ml), or UEA-1 alone. After the 24 hour incubation, supernatants were assayed for IL-1α (FIG. 18A) and IL-1β (FIG. 18B) by ELISA.

In the absence of TLR2 stimulation, no significant (p>0.05) enhancement of IL-1α (FIG. 18A) or IL-1β (FIG. 18B) was found in dendritic cells in response to PS particles targeted with UEA-1 compared to targeted particles.

Production of IL-1α by TLR2-stimulated dendritic cells was significantly increased (p<0.001) by targeting PS particles with UEA-1 (FIG. 18A). Similarly when dendritic cells were stimulated with Pam3CSK, PS particle-mediated IL-1β production was significantly increased (p<0.001) by targeting with UEA-1 (FIG. 18B). Thus, it appears that the enhancement of TLR-activated IL-1α and IL-1β production by dendritic cells stimulated with UEA-1-targeted PS particles is not dependent on TLR-4 activation, but can also be activated by stimulating TLR-2 with appropriate agonists before incubation with PS particles. This shows that PS particles and conjugated UEA-1 may synergize with other TLR agonists besides LPS to enhance IL-1α and IL-1β production.

### Example 16

### Targeting Particles to Dendritic Cells with Lectins Increases Particle-Driven IL-1α and IL-1β Cytokine Production

In order to determine whether other lectins influence TLR4-primed IL-1α and IL-1β cytokine production in vitro, the following experiment was undertaken.

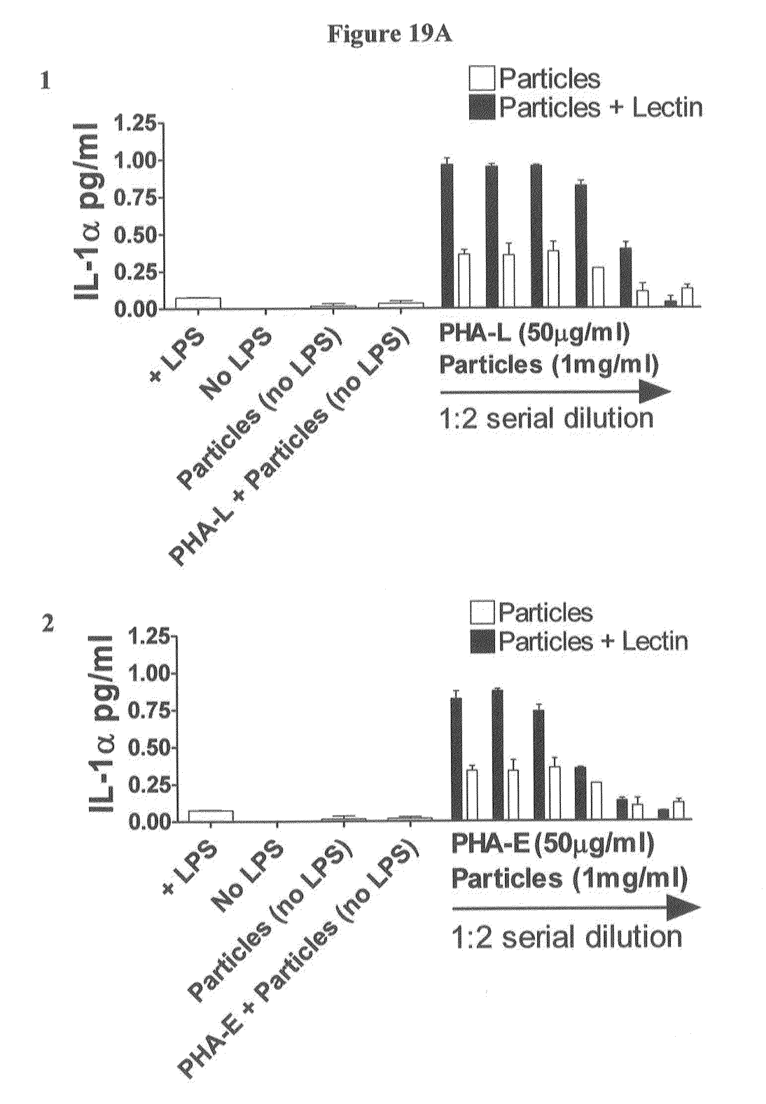

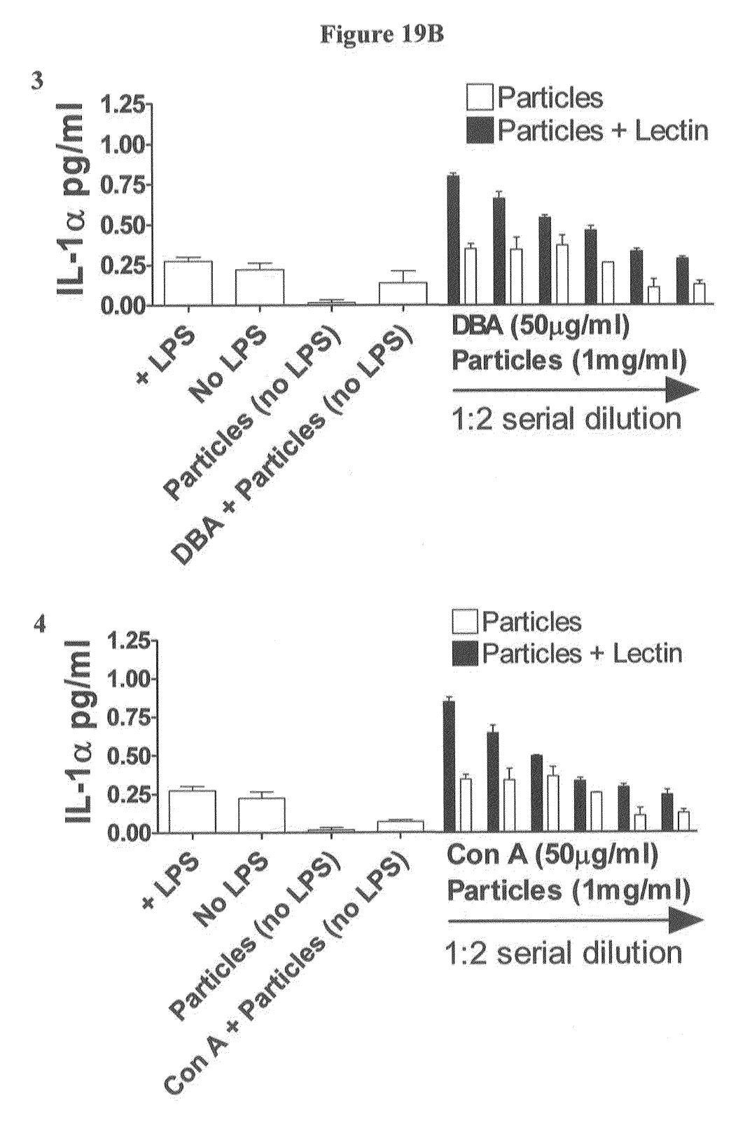

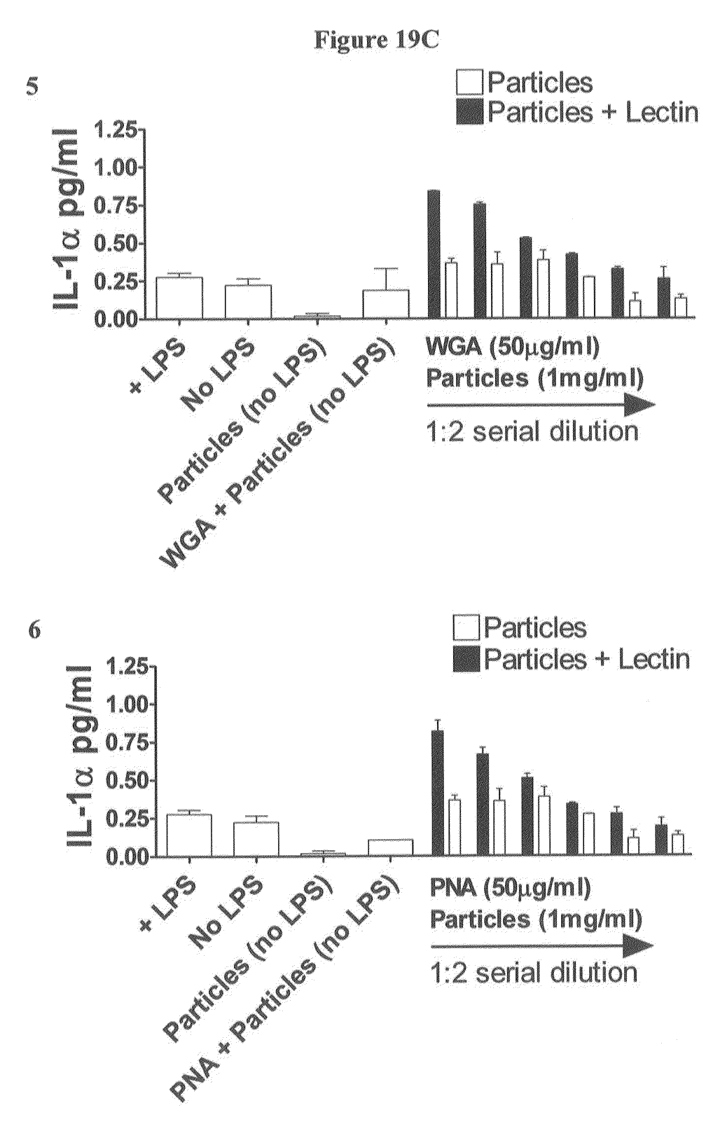

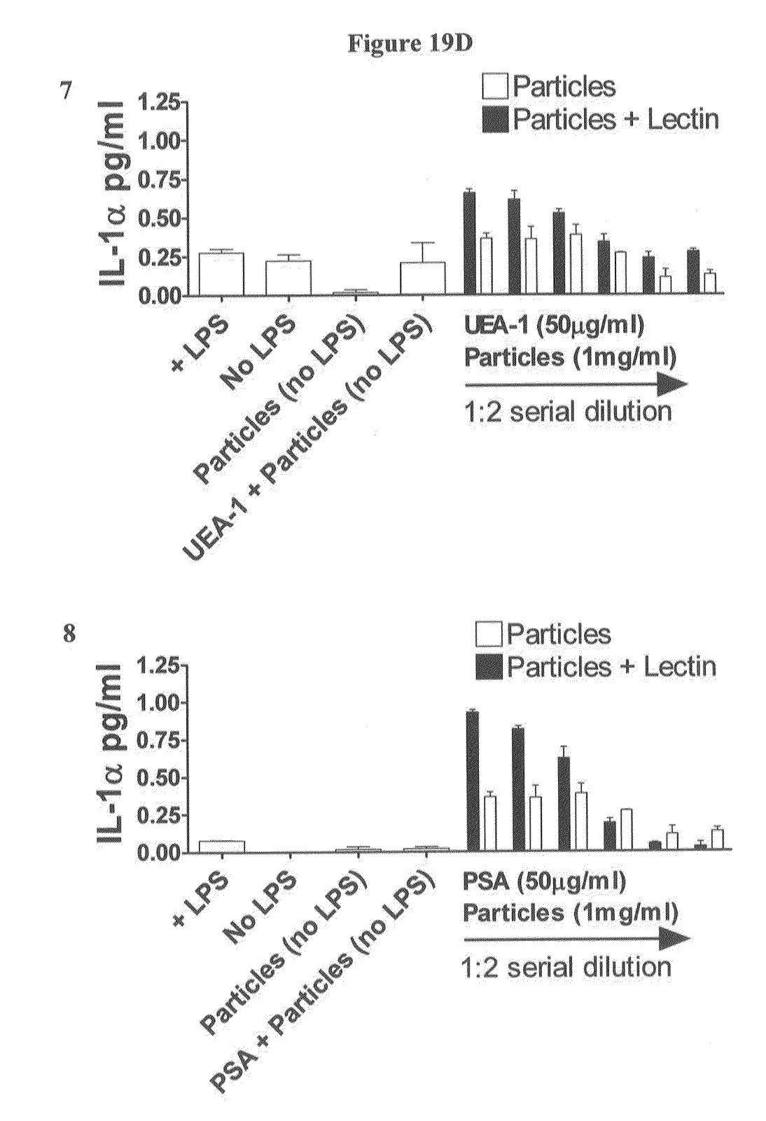

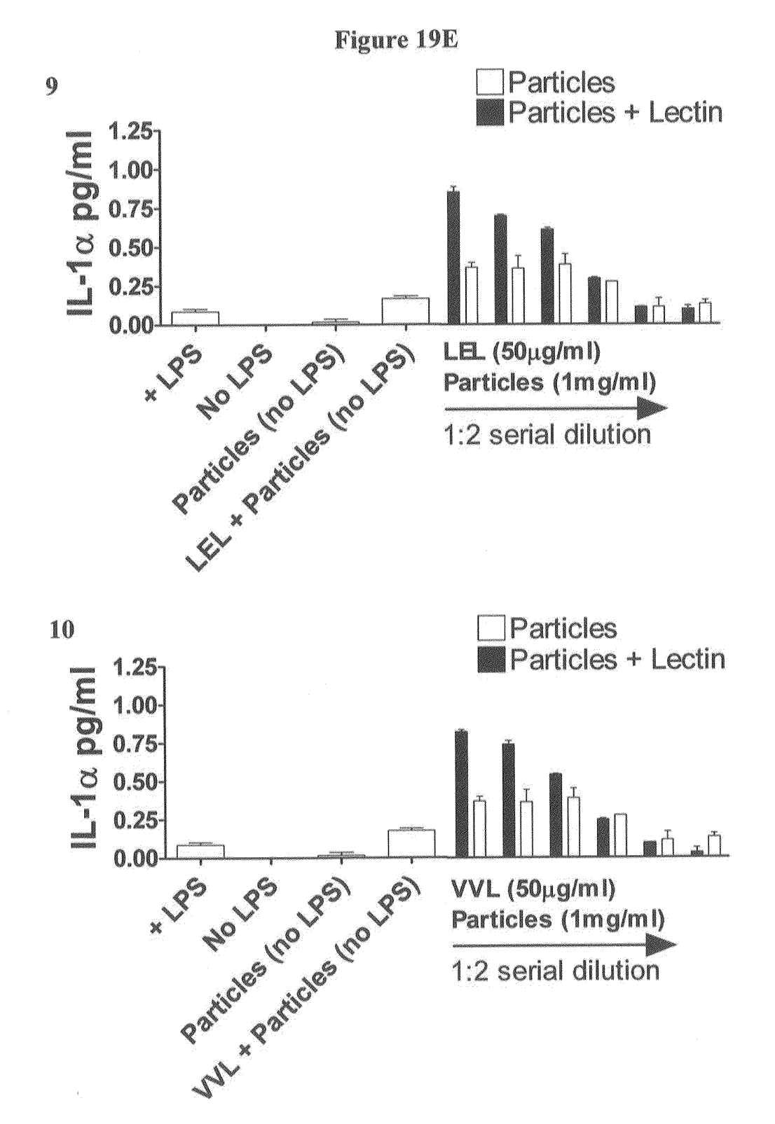

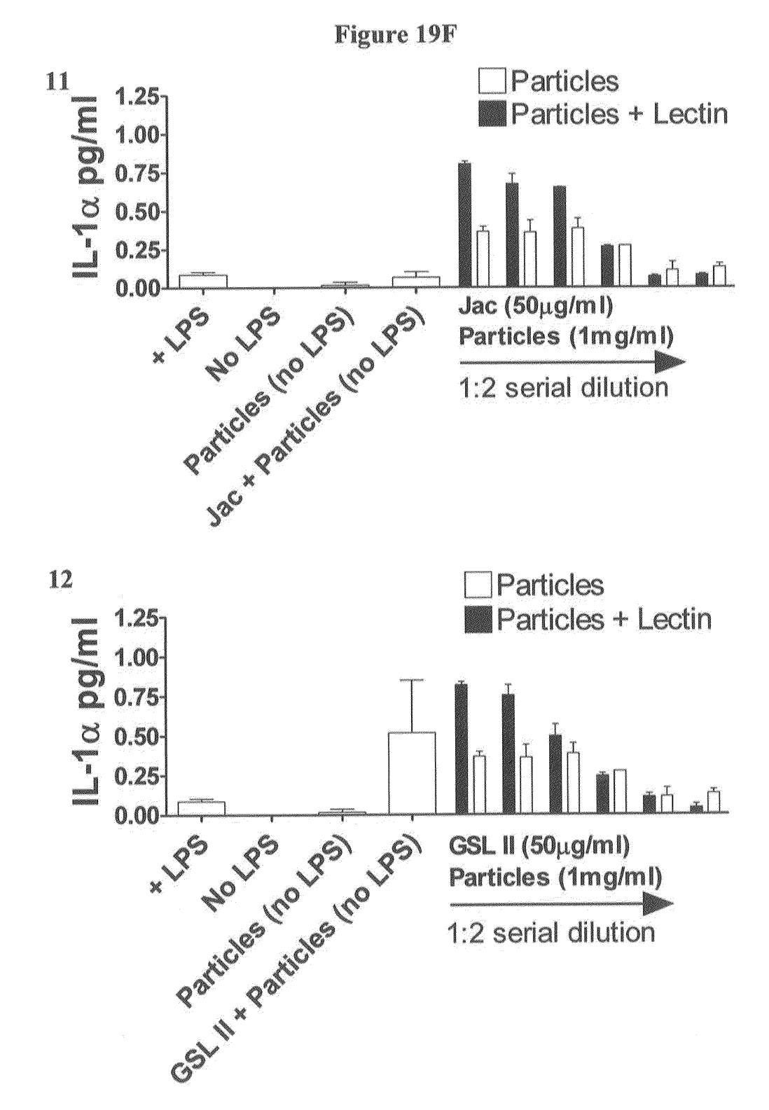

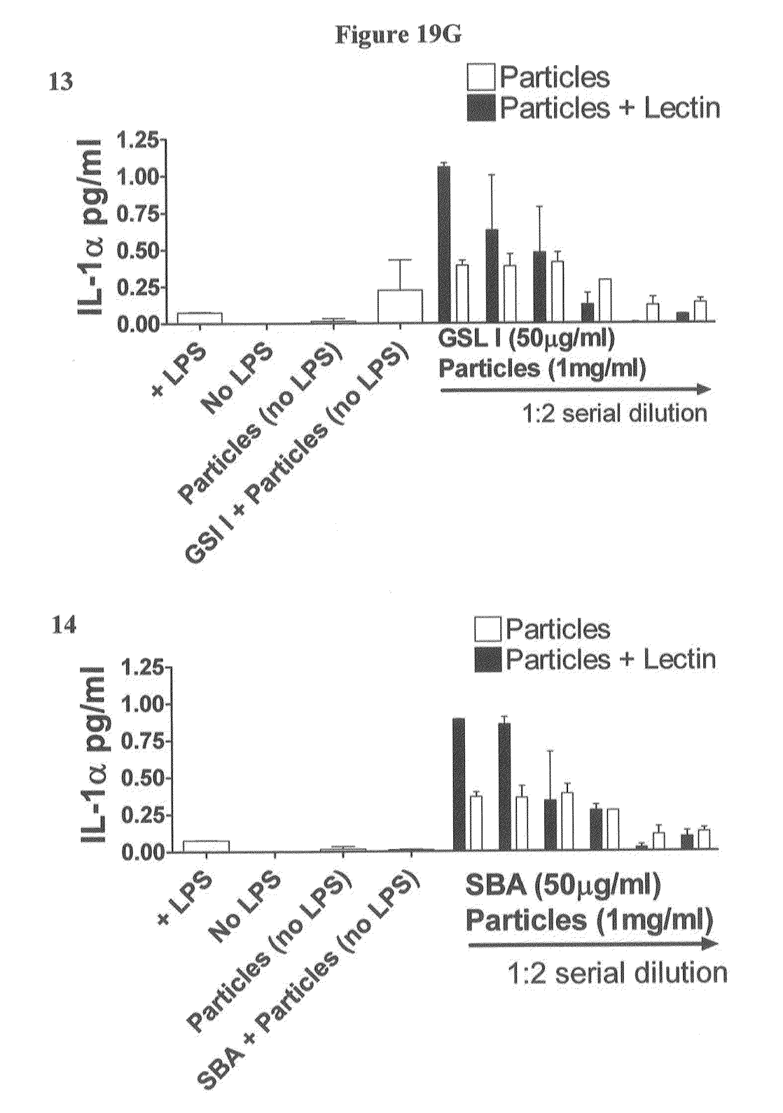

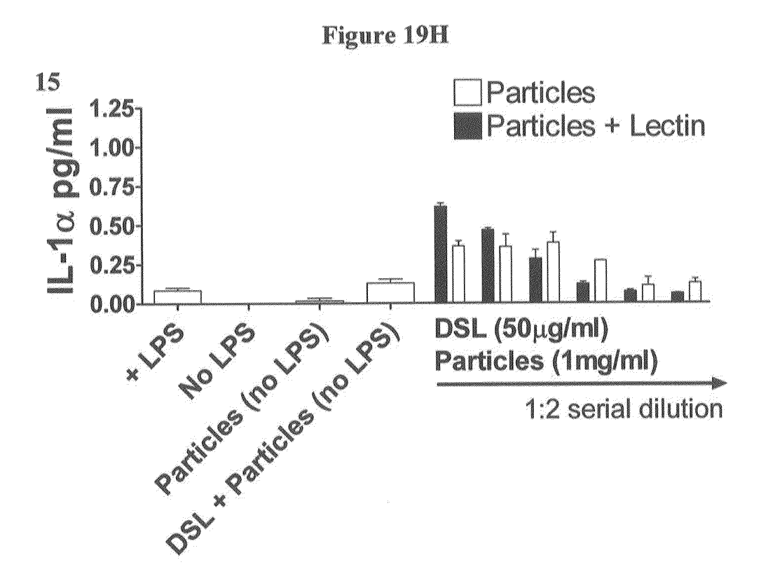

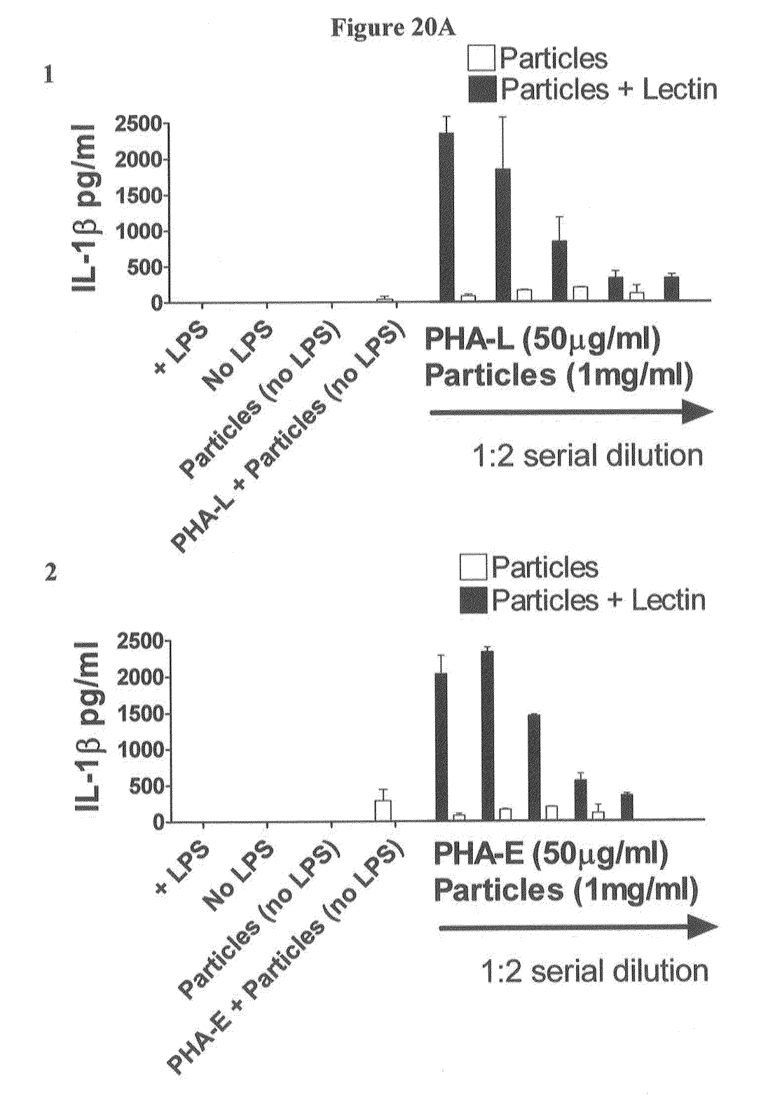

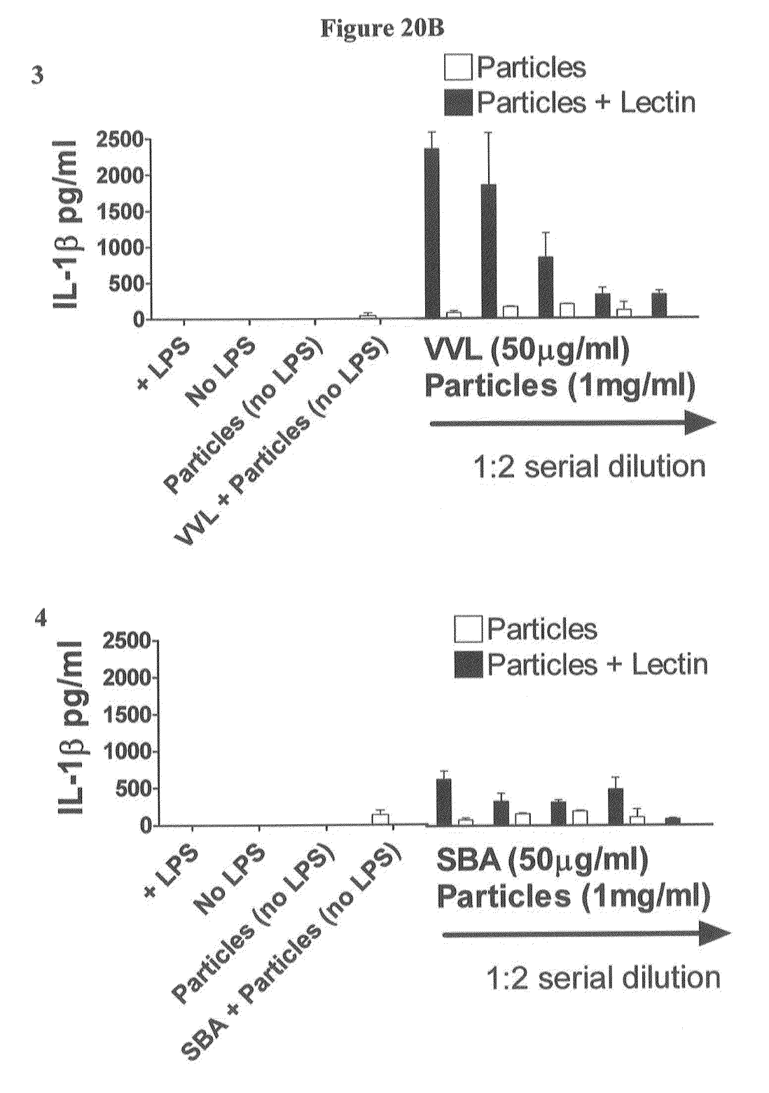

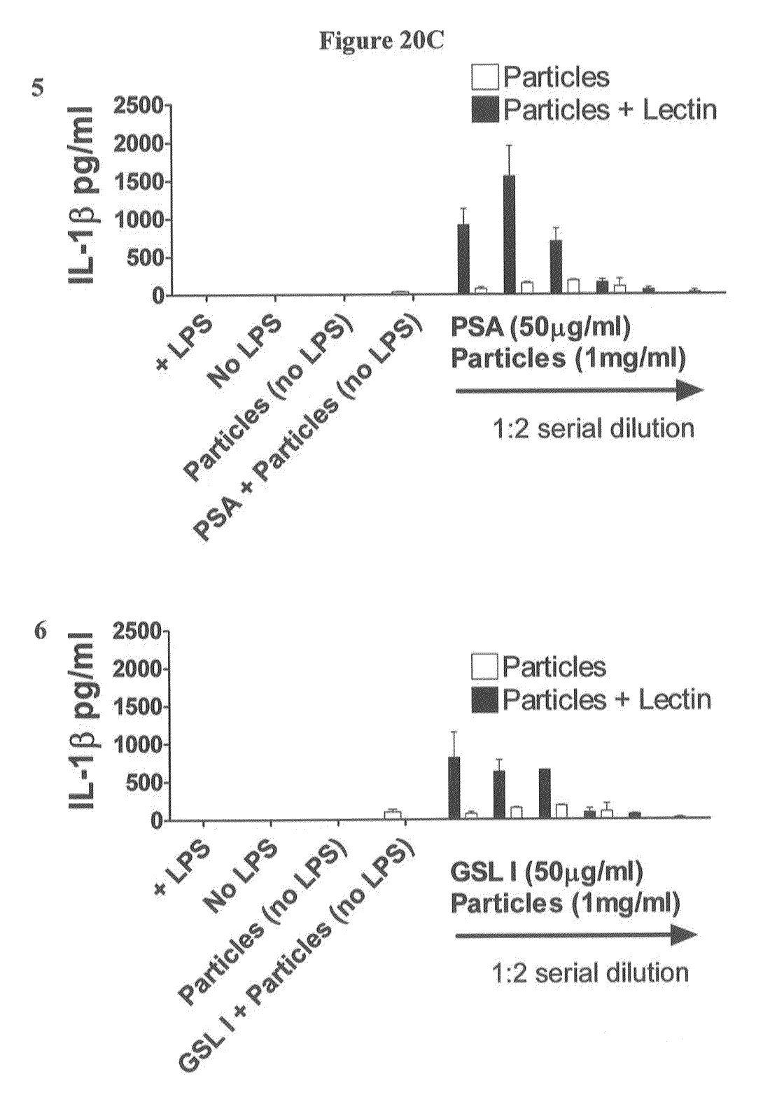

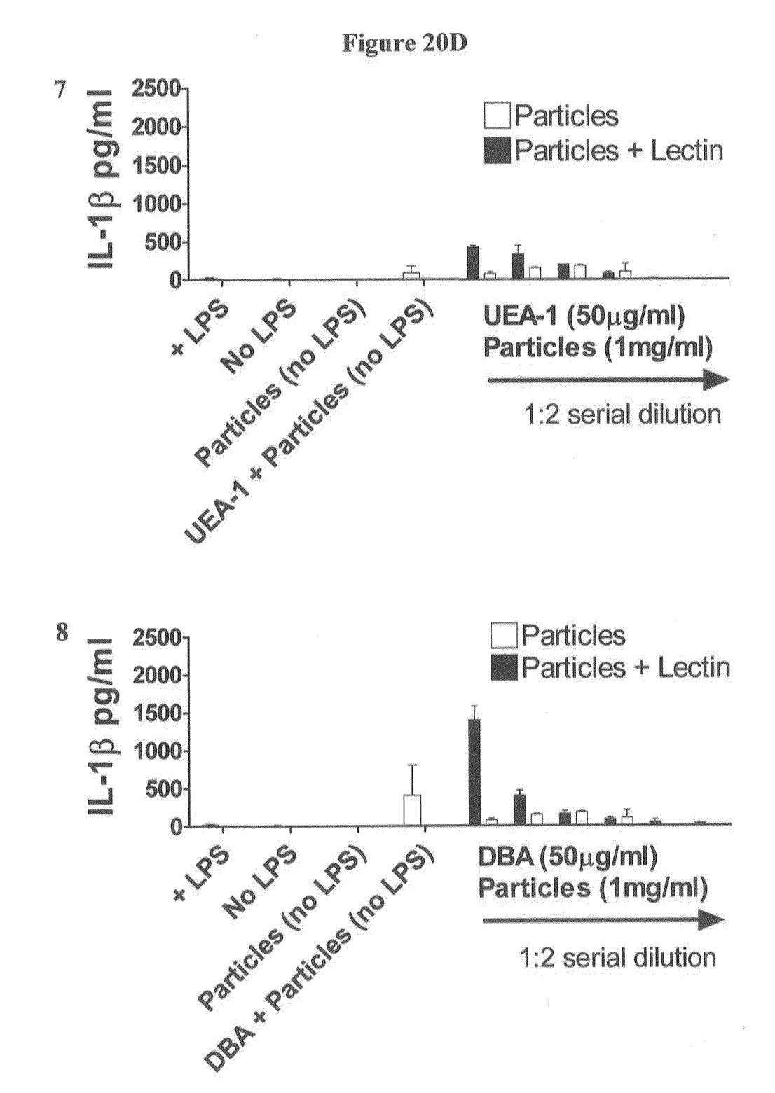

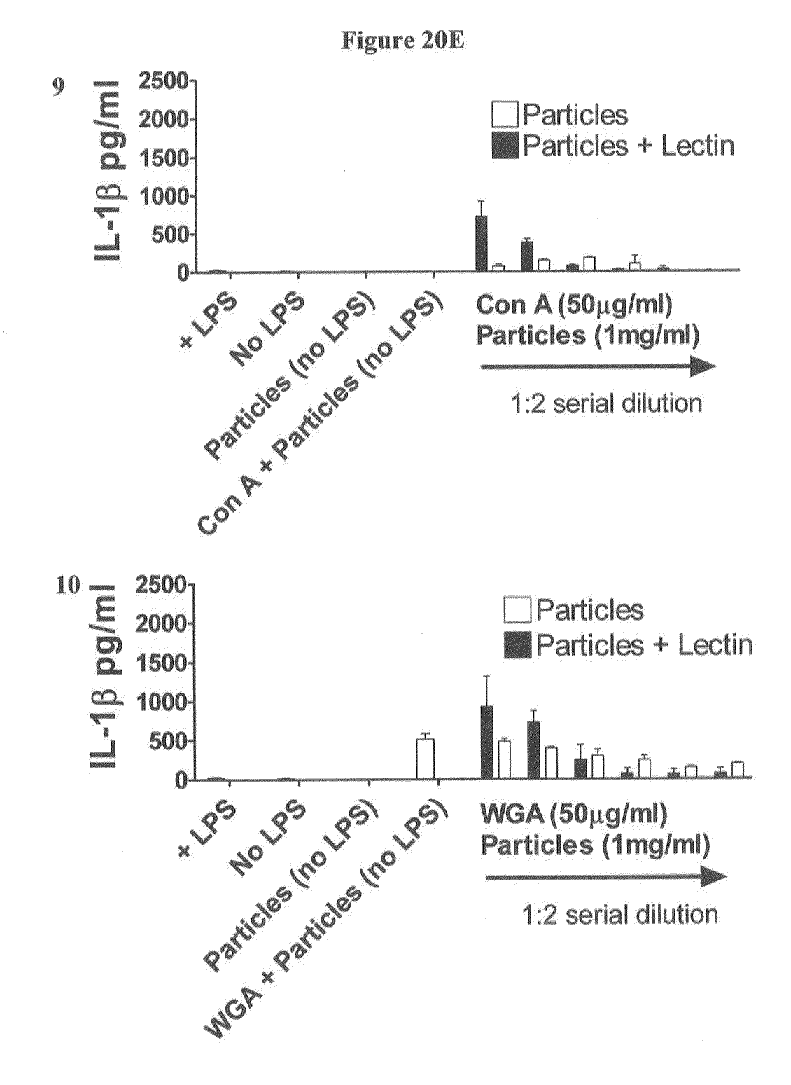

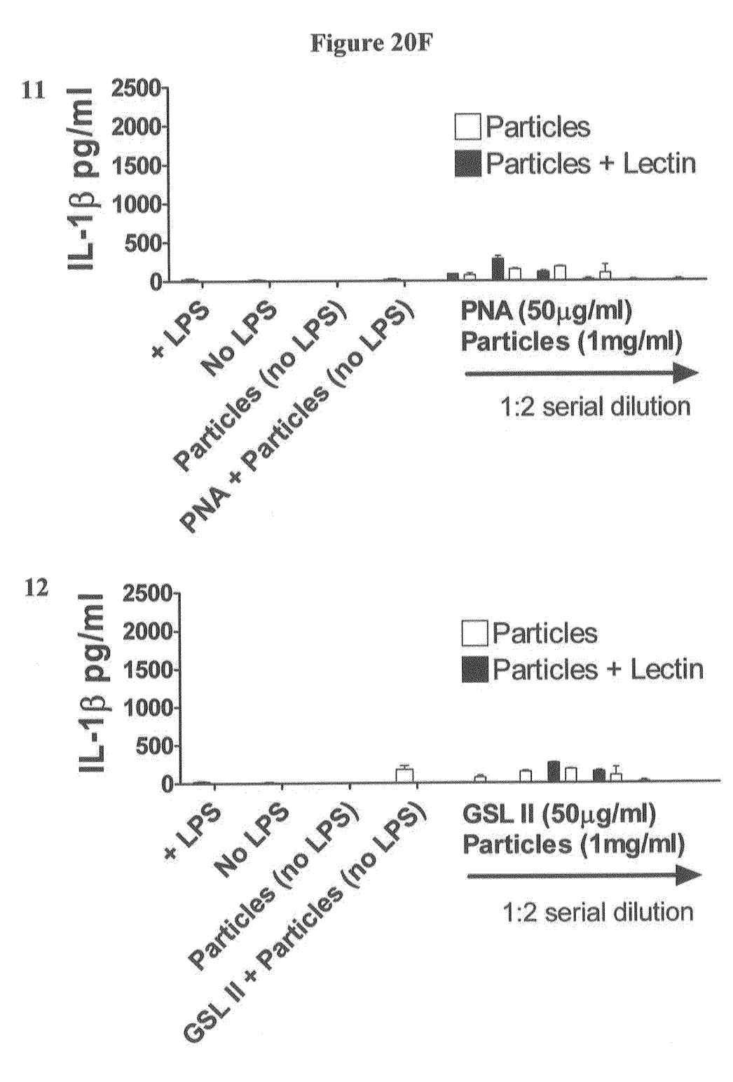

Murine C57BL/6 BMDCs (6.25×10⁵ cells/ml) were stimulated with LPS (1 ng/ml) for 6 hours or left unstimulated. After 6 hours, these cells were incubated with serially diluted SC-PS particles (31.254 ml to 1 mg/ml) or serially diluted SC-PS particles (31.25 μg/ml to 1 mg/ml) conjugated with biotinylated Con A (1.56 to 50 μg/ml), biotinylated DBA (1.56 to 50 μg/ml), biotinylated DSL (1.56 to 50 μg/ml), biotinylated GSL I (1.56 to 50 μg/ml), biotinylated GSL II (1.56 to 50 μg/ml), biotinylated Jac (1.56 to 50 μg/ml), biotinylated LEL (1.56 to 50 μg/ml), biotinylated PHA-E (1.56 to 50 μg/ml), biotinylated PHA-L (1.56 to 50 μg/ml), biotinylated PNA (1.56 to 50 μg/ml), biotinylated PSA (1.56 to 50 μg/ml), biotinylated SBA (1.56 to 50 μg/ml), biotinylated UEA-1 (1.56 to 50 μg/ml), biotinylated VVL (1.56 to 50 μg/ml) or biotinylated WGA (1.56 to 50 μg/ml). After 24 hour incubation, supernatants were assayed for IL-1α (FIGS. 19A-19H) and IL-1β (FIGS. 20A-20F) by ELISA.

As shown in FIGS. 19A-19H and 20A-20F, SC-PS particles conjugated with lectins increased the production of both IL-1α and IL-1β more efficiently than SC-PS particles alone. Each of the lectins tested increased cytokine production to some extent. Some of the lectins maintained increased cytokine production even at concentrations as low as 1.5625 μg/ml of lectin conjugated to 31.25 μg/ml of SC-PS particles.

Each of the lectins tested increased the production of IL-1α more efficiently than unconjugated SC-PS particles. PHA-L and PHA-E greatly increased IL-1α production and remained effective across four of the five serial dilutions (FIG. 19A; panels 1 and 2). DBA, Con A, WGA, PNA and UEA-1 increased IL-1α production across all five of the serial dilutions (FIGS. 19B-19D; panels 3, 4, 5, 6 and 7). Interestingly, DSL (FIG. 19H; panel 15) was the least efficient of the lectins tested even though DSL effectively targets BMDCs (see FIGS. 5A-5B and 7).

Each of the lectins tested increased the production of IL-1β more efficiently than unconjugated SC-PS particles, which only slightly increased IL-1β production above control levels. PHA-L, PHA-E and VVL greatly increased IL-1β production and remained effective across four serial dilutions (FIGS. 20A-20B; panels 1, 2 and 3). SBA increased IL-1β production to a lesser extent, but likewise remained effective across four serial dilutions (FIG. 20B; panel 4).

### Example 17

### The Enhancement of IL-1α Production by Dendritic Cells in Response to Lectin-Targeted Particles May not be Dependent on the NLRP3 Inflammasome

In order to determine whether the increase in IL-1α production achieved by lectin targeting of PS particles is dependent on the NLRP3 inflammasome, the following experiment was carried out.

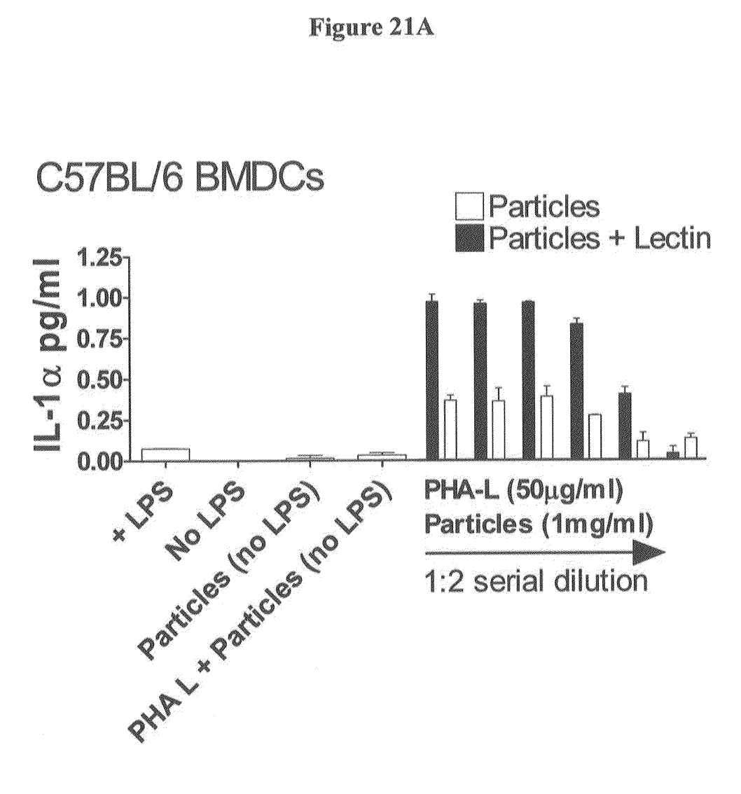

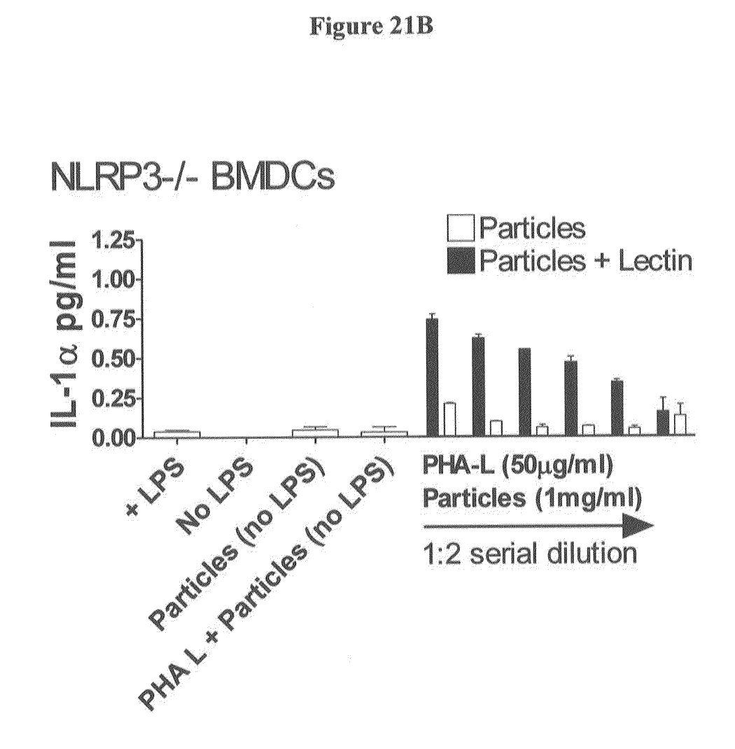

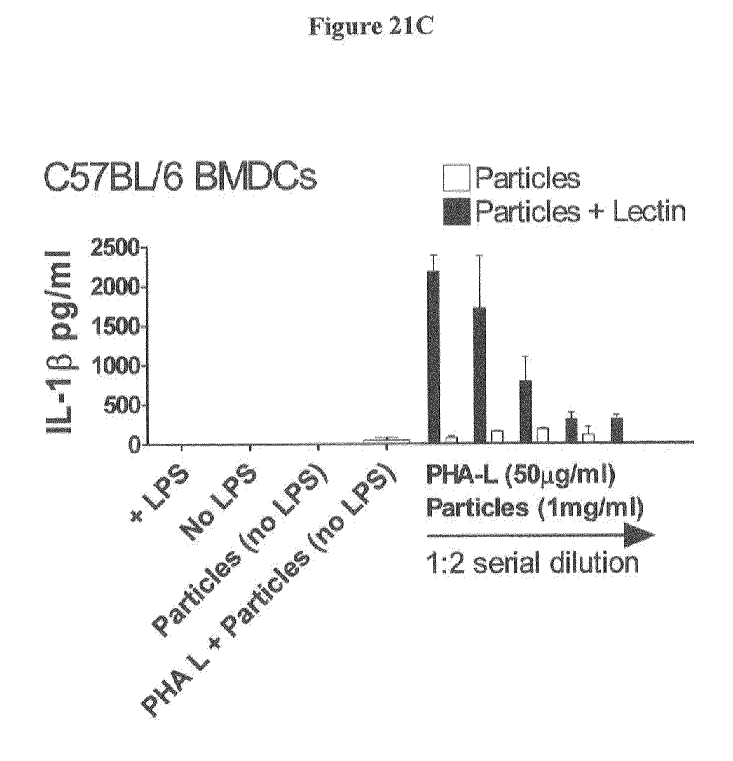

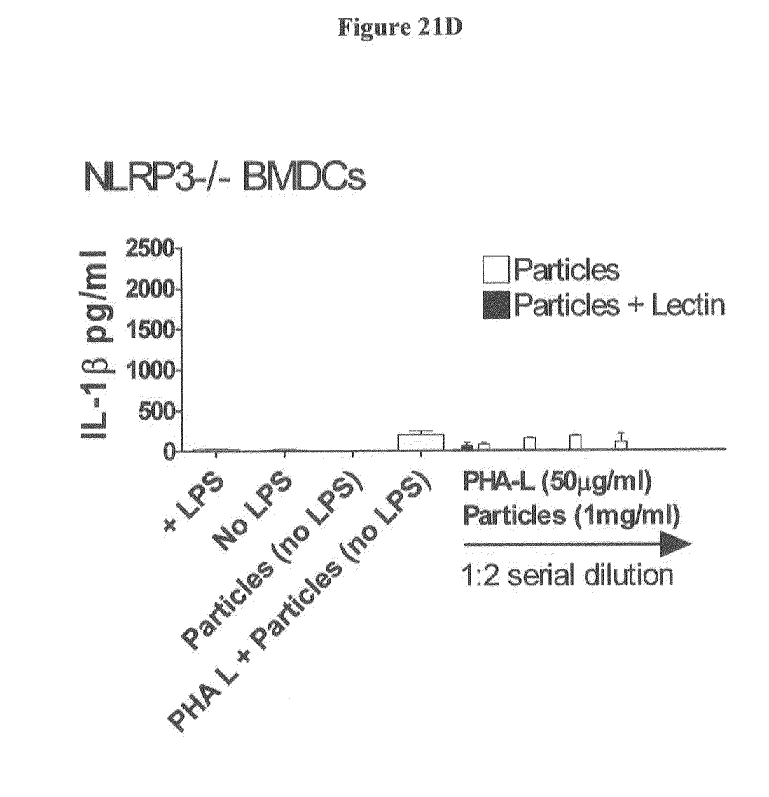

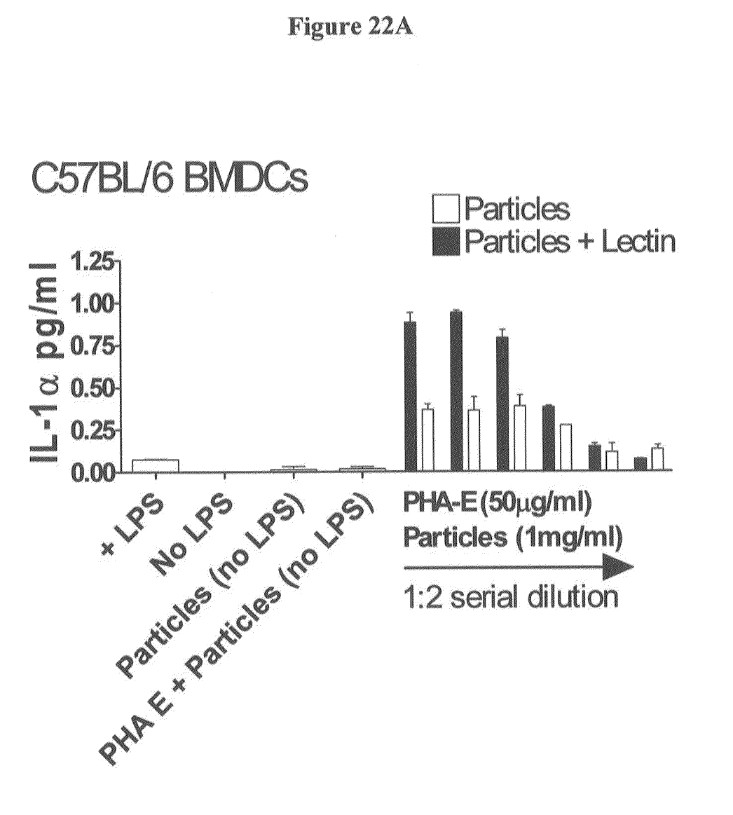

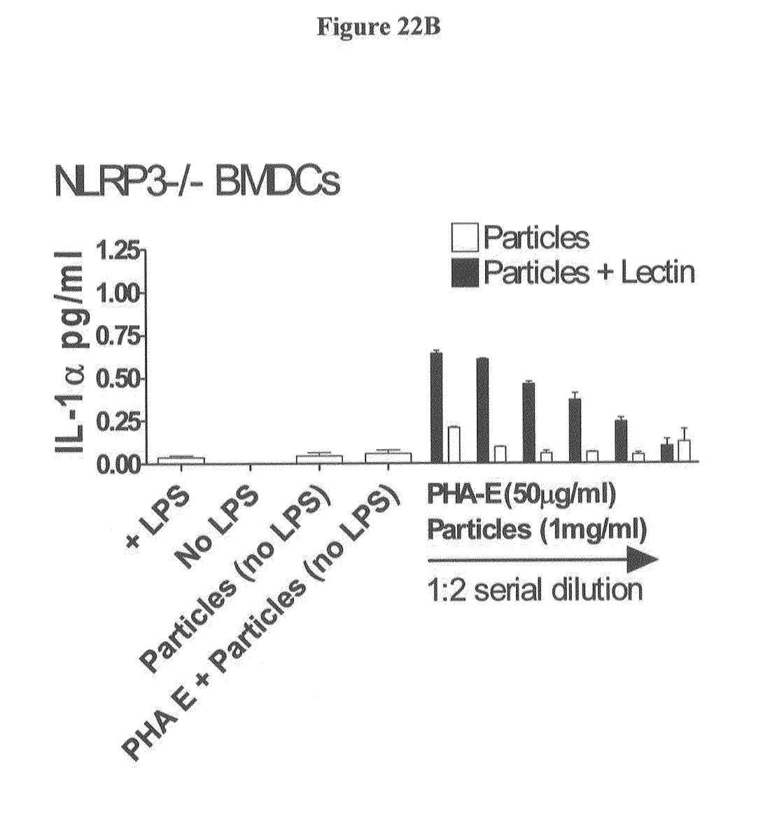

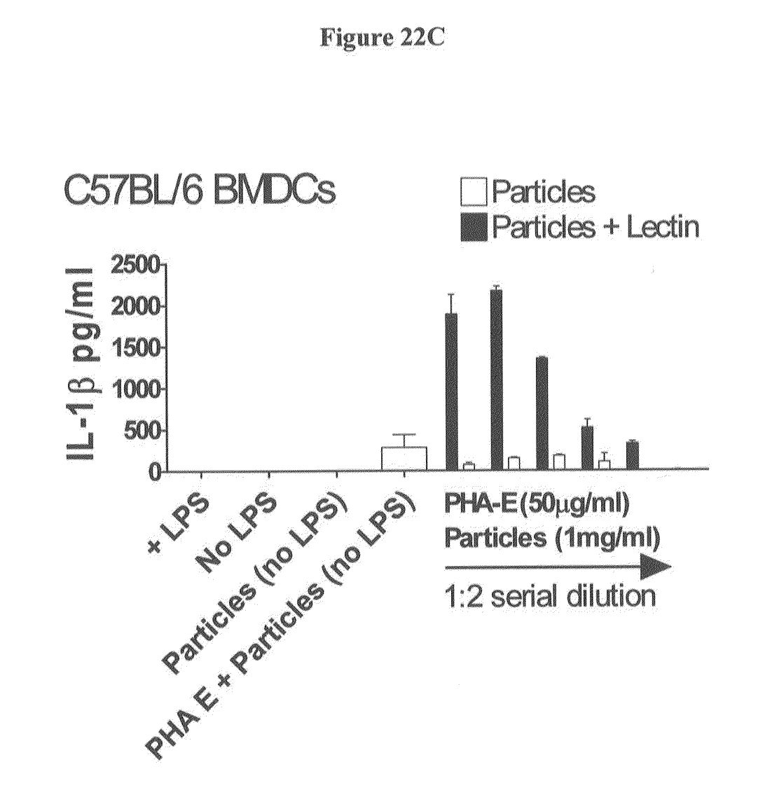

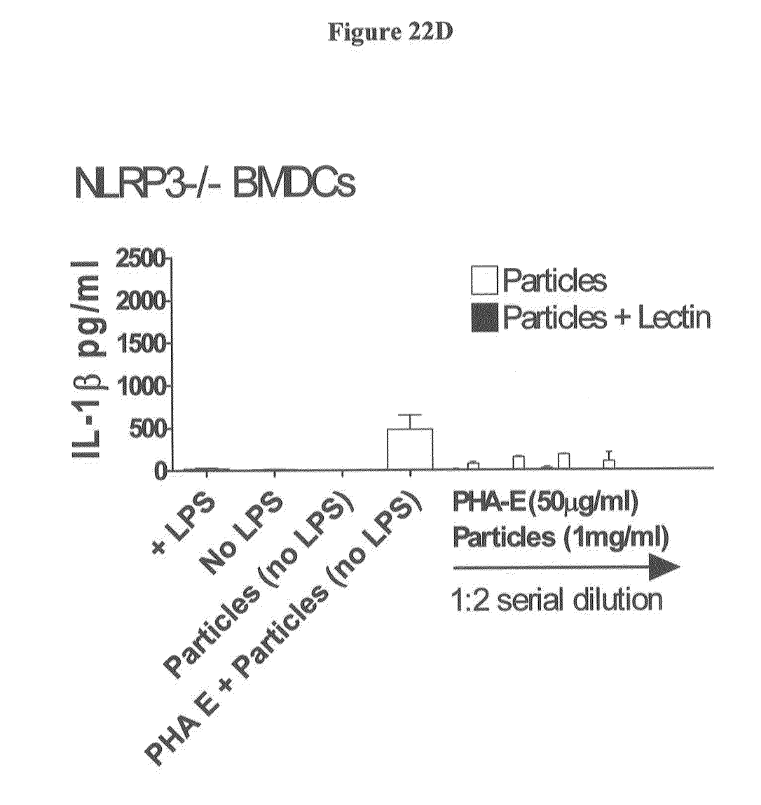

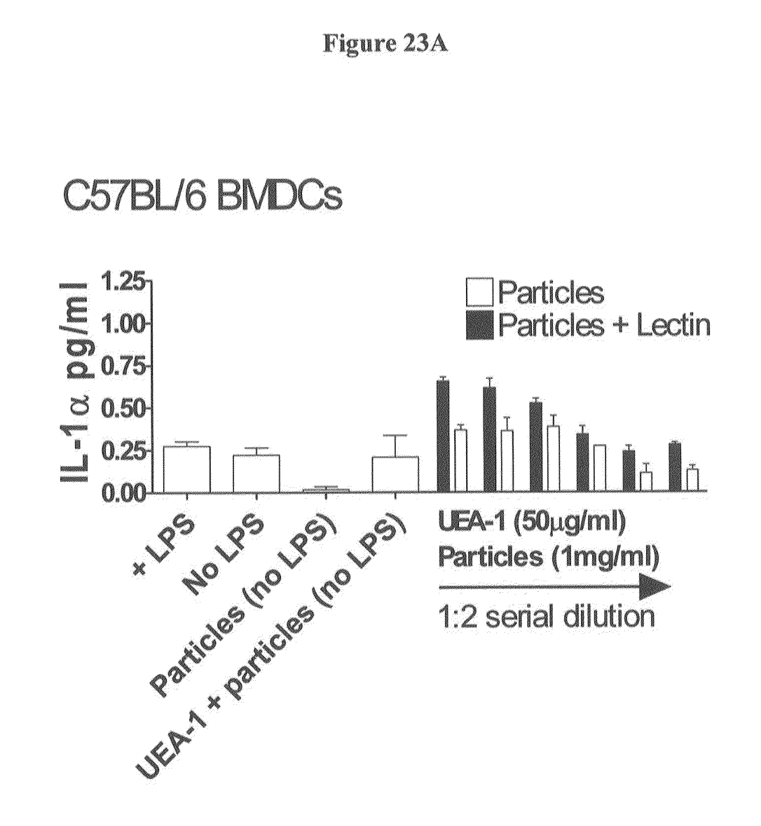

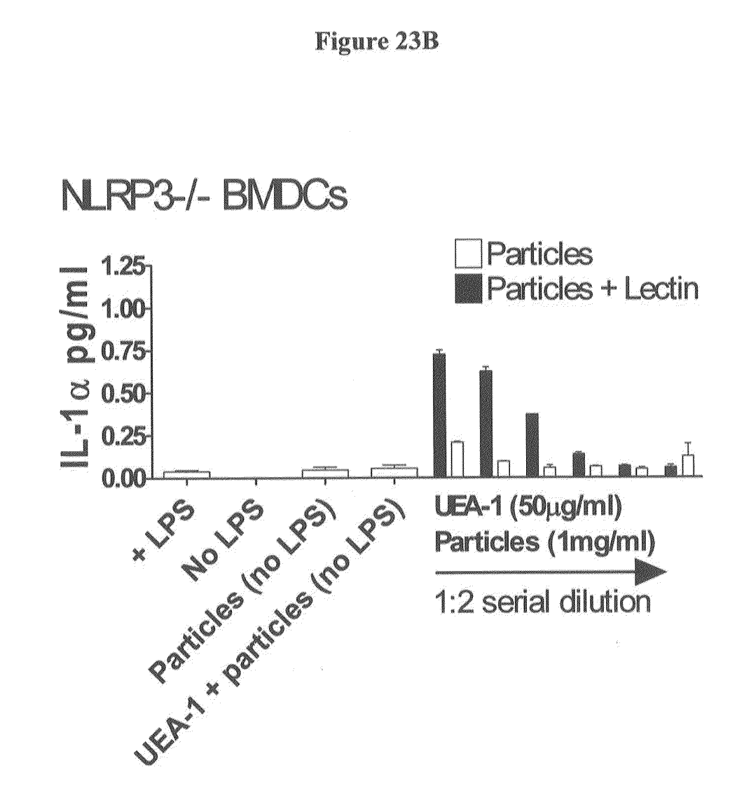

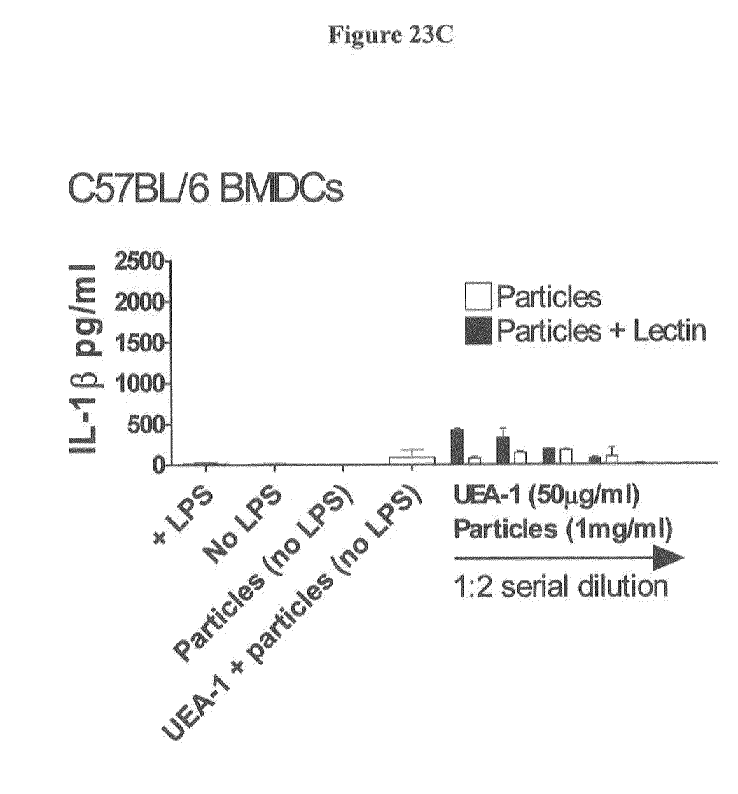

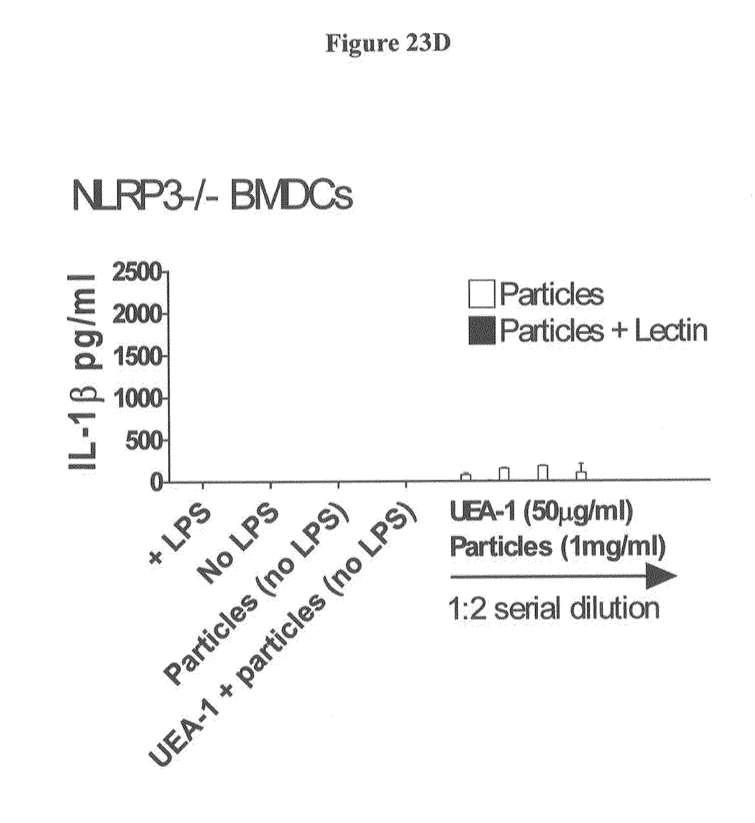

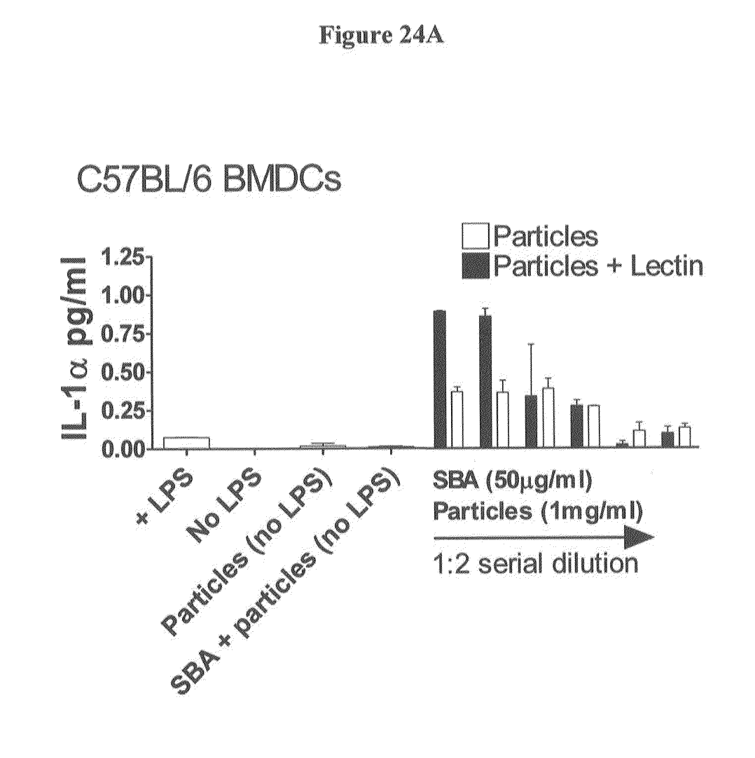

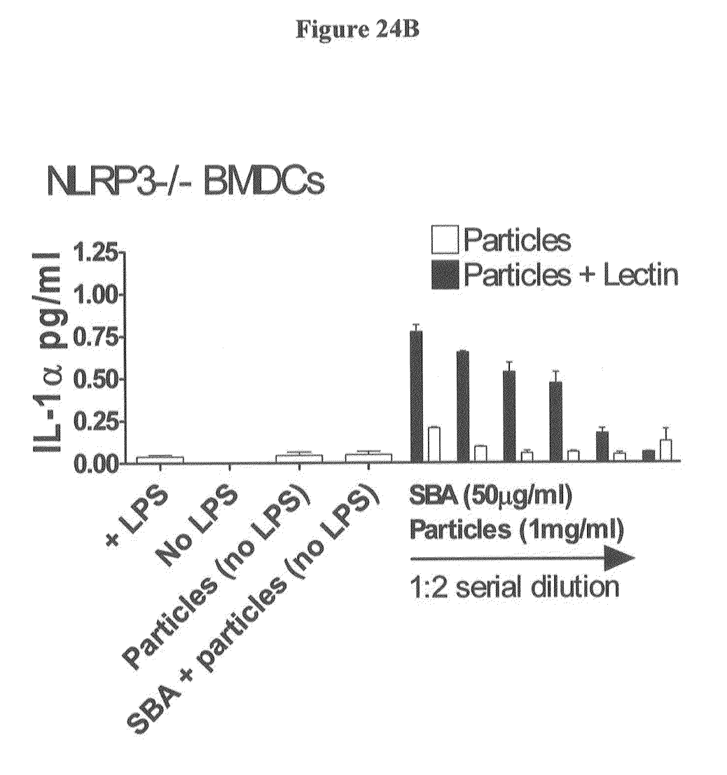

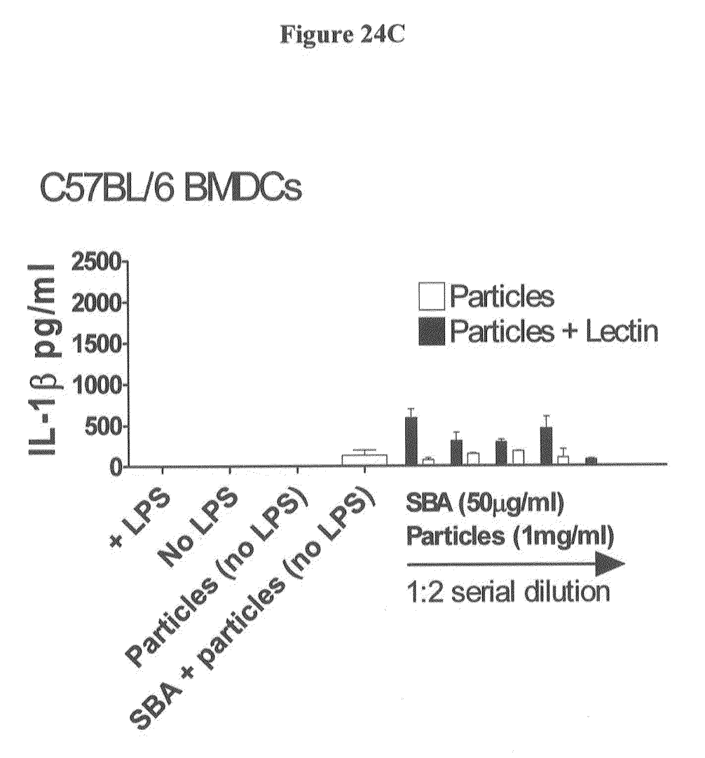

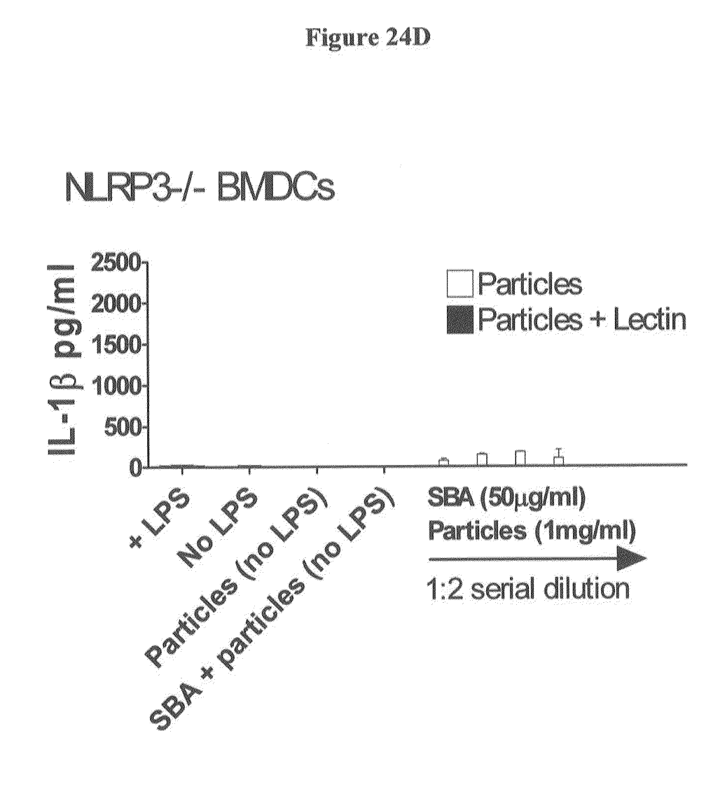

BMDCs (6.25×10⁵ cells/ml) from NLRP3^{-/-} and C57BL/6 mice were stimulated with LPS (1 ng/ml) for 6 hours or left unstimulated. After 6 hours, these cells were incubated with SC-PS particles (31.25 μg/ml to 1 mg/ml) or SC-PS particles (31.25 μg/ml to 1 mg/ml) conjugated with biotinylated PHA-E (1.56 to 50 μg/ml), biotinylated PHA-L (1.56 to 50 μg/ml), biotinylated SBA (1.56 to 50 μg/ml) or biotinylated UEA-1 (1.56 to 50 μg/ml). After 24 hour incubation, supernatants were assayed for IL-1α (FIGS. 21A, 21B, 22A, 22B, 23A, 23B, 24A and 24B) by ELISA.

As shown in FIGS. 21A, 21B, 22A, 22B, 23A, 23B, 24A and 24B, IL-1α production by BMDCs from NLRP3^{-/-} mice was reduced as compared to IL-1α production by BMDCs from C57BL/6 mice. However, IL-1α production was increased by stimulating dendritic cells from either wild-type C57BL/6 or NLRP3^{-/-} mice with targeted particles compared to untargeted particles, indicating that the lectin-mediated enhancement of IL-1α production may not be dependent on NLRP3.

### Example 18

### The Enhancement of IL-1β Production by Dendritic Cells in Response to Lectin-Targeted Particles is Dependent on the NLRP3 Inflammasome

In order to determine whether the increase in IL-1β production achieved by lectin targeting of PS particles is dependent on the NLRP3 inflammasome, the following experiment was carried out.

BMDCs (6.25×10⁵ cells/ml) from NLRP3^{-/-} and C57BL/6 mice were stimulated with LPS (1 ng/ml) for 6 hours or left unstimulated. After 6 hours, these cells were incubated with SC-PS particles (31.25 μg/ml to 1 mg/ml) or SC-PS particles (31.25 μg/ml to 1 mg/ml) conjugated with biotinylated PHA-E (1.56 to 50 μg/ml), biotinylated PHA-L (1.56 to 50 μg/ml), biotinylated SBA (1.56 to 50 μg/ml) or biotinylated UEA-1 (1.56 to 50 μg/ml). After 24 hour incubation, supernatants were assayed for IL-1β (FIGS. 21C, 21D, 22C, 22D, 23C, 23D, 24C and 24D) by ELISA.

As shown in FIGS. 21C, 21D, 22C, 22D, 23C, 23D, 24C and 24D, IL-1β production by BMDCs from NLRP3^{-/-} mice was minimal or absent as compared to IL-1β production by BMDCs from C57BL/6 mice. Small quantities of IL-1β were produced by BMDCs from NLRP3^{-/-} mice following incubation with SC-PS particles alone, but no appreciable IL-1β production occurred in cells treated with lectin-targeted particles, indicating that the lectin-mediated enhancement of IL-1β production is dependent on NLRP3.

### Example 19

### Western Blotting

Western Blots were used to determine the presence of active IL-1β in PS particle-stimulated BMDC supernatants.

Protein Extraction from Supernatants and Sample Preparation

C57BL/6 BMDCs were isolated and cultured as described above at a density of 6.25×10⁵ cells/ml in a 96 well U-bottomed plate in 200 μl complete RPMI 1640 medium per well. After 6 hours stimulation with either medium or a TLR agonist (LPS), cells were stimulated for a further 18 hours with either medium, SC-PS particles, SC-PS particles conjugated with UEA-1, SC-PS particles conjugated with UEA-1 mimetic, or UEA-1 alone and the supernatants were collected. 500 μl of each supernatant was added to a 1 ml Eppendorf tube and centrifuged at 14,000 rpm for 10 minutes at 4° C. to remove residual PS particles. Supernatants were transferred to fresh 1 ml Eppendorf tubes. 500 μl methanol and 100 μl chloroform was added to each supernatant, and the tubes were vortexed. These samples were centrifuged at 13,000 rpm for 3 minutes at 4° C. A white layer formed at the interface between the lower phase (chloroform) and the upper phase (methanol/H₂O). The upper phase was removed until the white layer was accessible. 500 μl methanol was added, and the tube vortexed. Samples were centrifuged again at 13,000 rpm for 3 minutes at 4° C. All the supernatant was then removed and the protein pellet at the bottom was allowed to air-dry until it changed from white to a yellow/brown. Once dry, the pellet was resuspended in 50 μl sample buffer (65 mM Tris pH 6.8, 2% SDS (w/v), 10% glycerol, 0.1% bromophenol blue, 50 mM DDT). Samples were then boiled in a 95-100° C. heating block for 5 minutes before being placed on ice.

SDS Polyacrylamide Gel Electrophoresis (SDS-PAGE)

The Resolving gel (Table 3) was prepared and poured between two glass plates. The gel was allowed to set before the addition of the Stacking gel (Table 3) and a comb inserted between the plates. Once the stacking gel was set, 1× running buffer (15 g Tris base, 72 g glycine and 5 g SDS in 1 L dH₂O, adjusted to pH 8.3) was added to the rig and the comb removed. 4 μl of a molecular weight ladder was added to the first lane and 10 μl of sample added to subsequent appropriate lanes. The gel was run at 90V until the samples had reached the separating gel and then the voltage was increased to 120V. The apparatus was stopped when the samples had reached the bottom of the gel.

TABLE 3
SDS-PAGE Gels

| | 4% Stacking Gel | | 15% Resolving Gel | |
|---|---|---|---|---|
| dH₂0 | 12.2 | ml | 10.03 | ml |
| 30% bis-acrylamide mix | 2.6 | ml | 8.33 | ml |
| 0.5M Tris pH 6.8 | 5 | ml | — | |
| 1.5M Tris pH 8.8 | — | | 6.25 | ml |
| 10% ammonium persulphate | 100 | μl | 150 | μl |
| 10% SDS | 200 | μl | 250 | μl |
| TEMED | 20 | μl | 12.5 | μl |

Transfer of Proteins to Nitrocellulose Membrane

Proteins from the gel were transferred to a nitrocellulose membrane using a semi-dry transfer system. The gel was carefully removed from between the two glass plates and kept moist in transfer buffer (0.19 g Tris base, 4.32 g glycine, 60 ml methanol, 0.15 g SDS in 240 ml dH₂O, adjusted to pH 8.3). The gel was placed on the nitrocellulose membrane between layers of moist filter paper. Any air bubbles were removed from the layers of the “transfer sandwich” by gently rolling over with a 10 ml pipette. The “transfer sandwich” was then placed in the transfer apparatus and a current of 300 mA applied for 1 hour.

Detection

After transfer of protein to nitrocellulose membrane, the membranes were blocked for non-specific binding in 10% milk blocking buffer for 1 hour at room temperature on a rocker. The blot was then washed in PBS-Tween (6×5 minutes). The blot was then incubated with the primary antibody (anti-IL-1β; R&D Systems, Inc., Minneapolis, Minn.) according to the manufacturer's specifications ( 1/500 dilution in 1×PBS with 3% BSA) for 2 hours at room temperature on a rocker. The blot was again washed in PBS-T (6×5 minutes). Secondary antibody (anti-rat IgG peroxidase conjugate; 1/2000 dilution in 1×PBS with 3% BSA) was added to the blot and incubated for 1 hour at room temperature on a rocker. A final wash with PBS-T was performed (6×5 minutes). Chemiluminescence substrate solutions A (50 μl luminol, 22 μl p-coumaric acid and 500 μl Tris pH 8.8 in 4.5 ml dH₂O) and B (500 μl Tris pH 8.8 and 3 μl H₂O₂ in 4.5 ml dH₂O) were mixed together and applied for 2 minutes and the blot developed.

### Example 20

### Targeting of Polystyrene Particles with UEA-1 or UEA-1 Mimetic Increases Particle-Induced Secretion of Processed IL-1β by Dendritic Cells

Having shown that both UEA-1 and UEA-1 mimetic induce increased IL-1β production when conjugated to PS particles compared to PS particles alone in LPS-primed cells, western blots were performed on supernatants from cells to ascertain if the IL-β that is secreted is pro or active IL-1β.

Murine C57BL/6 BMDCs (6.25×10⁵ cells/ml) were stimulated with LPS (1 ng/ml) for 6 hours or left unstimulated. After 6 hours, the cells were incubated with SC-PS particles alone, SC-PS particles conjugated with UEA-1, SC-PS particles conjugated with UEA-1 mimetic or UEA-1 alone. After incubation, protein was extracted from supernatants and analyzed by western blot for active IL-1β at 17 kDa.

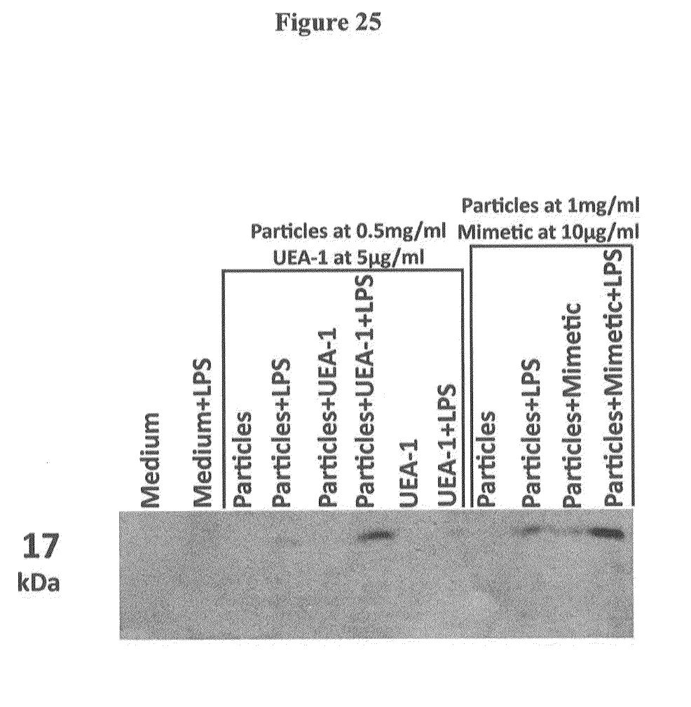

Stimulating dendritic cells with LPS and PS particles induced the production of active IL-1β (17 kDa). This active IL-1β (3 was increased by targeting the particles with either UEA-1 or UEA-1 mimetic (FIG. 25).

Thus, it appears that particles targeted to dendritic cells with UEA-1 or UEA-1 mimetic enhance the production of active IL-1β in vitro.

### Example 21

### Immunization Protocols for In Vivo Studies

Determining Adjuvant Activity of Particles

Five groups of 6-8 week old female BALB/c mice (five mice per group) were i.p, immunized on day 0 with a total volume of 200 μl of vaccine. All ovalbumin (OVA) used was endotoxin-free. The groups were:

Dulbecco's PBS

OVA only (50 μg/mouse)

OVA adsorbed onto PS particles (OVA-loaded PS particles)

UEA-1 adsorbed onto OVA-loaded PS particles

UEA-1 mimetic absorbed onto OVA-loaded PS particles

On day 34, blood was collected from the tail vein of each mouse and used to measure serum antibody titres. The following day, mice were i.p. immunized with an identical series of booster vaccinations as on day 0. Mice were sacrificed on day 42 by cervical dislocation, and cells were harvested.

Spleens were removed from the mice, and single cell suspensions prepared as described above. Peritoneal lavages were also performed. Cells were plated onto 96 well U-bottomed plates at cell densities of 2×10⁶ cells/ml for splenocytes and 1×10⁶ cells/ml for peritoneal lavage cells in 200 μl of complete RPMI 1640 medium per well.

Determining Whether UEA-1 Targeting is Mediated by NLRP3

Four groups of female C57BL/6 (WT) and NLRP3^{-/-} mice (five mice per group) were intranasally immunized on days 0, 14 and 28 with a total volume of 20 μl of vaccine. All ovalbumin (OVA) used was endotoxin-free. The groups were:

Dulbecco's PBS

OVA only (10 μg)

OVA attached to SC-PS particles

OVA attached to SC-PS particles loaded with UEA-1 mimetic (10 μg)

On day 35, the mice were sacrificed by cervical dislocation, blood was collected and used to measure serum antibody titres and cells were isolated from both the spleen and the mediastinal lymph nodes.

Determining Whether the UEA-1 Targeting Effect is Observed Following Intranasal Immunization with a Staphylococcus aureus Antigen

Four groups of female BALB/c mice (five mice per group) were intranasally immunized on days 0, 14 and 28 with a total volume of 20 μl of vaccine. The groups were:

Dulbecco's PBS

Clumping factor A (ClfA) only (10 μg)

ClfA attached to SC-PS particles

Clf A attached to SC-PS particles loaded with UEA-1 mimetic (10 μg)

On day 35, the mice were sacrificed by cervical dislocation, blood was collected and used to measure serum antibody titres and cells were isolated from both the spleen and the mediastinal lymph nodes.

Determining Whether Immune Responses can be Selectively Activated by Targeting Antigens with Different Plant Lectins

Seven groups of female BALB/c mice (five mice per group) were i.p. immunized on day 0 with a total volume of 200 μl of vaccine. The groups were:

Dulbecco's PBS

ClfA only (1 μg)

ClfA (1 μg) attached to SC-PS particles (100 μg)

Clf A attached to SC-PS particles loaded with UEA-1 (10 μg)

Clf A attached to SC-PS particles loaded with UEA-1 mimetic (10 μg)

Clf A attached to SC-PS particles loaded with PHA-L (10 μg)

Clf A attached to SC-PS particles loaded with SBA (10 μg)

On day 14, the mice were sacrificed by cervical dislocation, blood was collected and used to measure serum antibody titres and cells were isolated from both the spleen and the peritoneal cavity (peritoneal lavage).

Measuring Antigen-Specific Cytokine Responses

Cells were stimulated in vitro with PBS, endotoxin-free OVA (50 μg/ml, 100 μg/ml, 500 μg/ml), phorbol myristate acetate (PMA, 25 ng/ml) combined with anti-CD3 (1 μg/ml) or anti-CD3 alone (0.5 μg/ml). Cells were incubated with antigen for 3 days. Supernatants were then removed and IL-5, IL-10, IL-17 and IFN-γ cytokine concentrations were determined by ELISA.

Antigen-Specific Antibody Quantification ELISA

After immunization, tail bleed serum samples were collected from the mice and their antibody titres measured by ELISA. The following antigen-specific antibody titres were measured by immunoassay: IgG, IgG1 and IgG2a.

Standard Antigen-Specific Antibody Cytokine ELISA Protocol

Antigen-specific IgG and IgG subtypes were measured by coating 96-well medium binding plates with 50 μl/well of OVA antigen (50 μg/ml) in sodium carbonate buffer (4.2 g NaHCO₃ and 1.78 g Na₂CO₃ in 500 ml dH₂O, adjusted to pH 9.5). Plates were incubated for 2 hours at 37° C. Plates were then washed with PBS-T (×3) and tapped dry. Plates were blocked with 200 μl/well of 10% milk (5 g skimmed milk powder in 50 ml 1×PBS) for 2 hours at room temperature. Plates were again washed in PBS-T (×3) and tapped dry. Serum samples were diluted 1:100 in 1×PBS and added to the plate and serially diluted (1:2) across and plates incubated overnight at 4° C. PBS-T washes were again performed (×3) and tapped dry. Bound antibody was detected by adding 50 μl/well of anti-IgG ( 1/5,000 in 1×PBS; Sigma-Aldrich, St. Louis, Mo.), anti-IgG1 ( 1/4,000 in 1×PBS; BD Pharmingen, San Diego, Calif.) or anti-IgG2a ( 1/4,000 in 1×PBS; BD Pharmingen, San Diego, Calif.) detection antibody. Plates were incubated for 1 hour at 37° C. in the dark. After incubation plates were again washed in PBS-T (×3) and tapped dry. Extravidin-peroxidase (1:750 in 1×PBS) was added to the plates at 50 μl/well for 30 minutes in the dark. A final wash was performed with PBS-T (×3) and tapped dry. 50 μl of substrate solution was added to each well. After the colour reaction had occurred, the reaction was stopped with 25 μl/well of 1 M H₂SO₄, and the optical density values were obtained using a Multiskan® FC (Thermo Fisher Scientific, USA) microplate photometer.

Data Analysis

Data was analyzed using Prism® software (GraphPad Software, Inc., La Jolla, Calif.). Cytokine concentrations were compared by one-way ANOVA. Where significant differences were found, the Tukey-Kramer multiple comparisons test was used to identify differences between individual groups. Differences were considered significant when p<0.05. Error bars represent the standard error of the mean (SEM).

### Example 22

### Targeting Antigen-Loaded Particles with UEA-1 or Mimetic Did not Significantly Increase Antigen-Specific IgG Antibody Responses In Vivo

In order to determine whether immunisation with antigen-loaded PS particles targeted with UEA-1 or UEA-1 mimetic enhances the antigen-specific humoral response to that antigen, the following in vivo study was conducted.

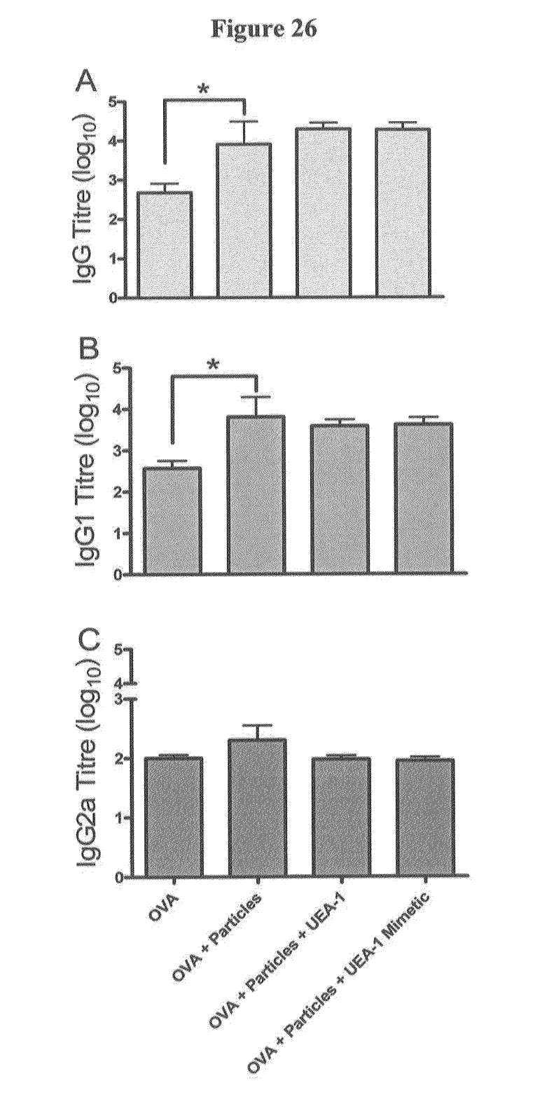

Five groups of BALB/c mice were i.p. immunized once (0 days) with OVA, OVA-loaded PS particles, UEA-1 adsorbed onto OVA-loaded PS particles or UEA-1 mimetic adsorbed onto OVA-loaded PS particles. Anti-OVA total IgG (FIG. 26A), IgG1 (FIG. 26B) and IgG2a (FIG. 26C) serum antibody titres were determined by ELISA on tail bleed serum samples recovered 34 days after initial immunization. Results are mean (±SE) endpoint titres for 5 mice per experimental group.

Total IgG

Mice immunized with OVA-loaded PS particles, had significantly (p<0.05) increased IgG titres compared to mice immunized with OVA alone (FIG. 26A). OVA-loaded PS particles targeted with UEA-1 or UEA-1 mimetic did not significantly enhance serum IgG antibody titres as compared to OVA-loaded PS particles alone (FIG. 26A).

IgG1

Mice immunized with OVA-loaded PS particles, had significantly (p<0.05) increased IgG1 titres compared to mice immunized with OVA alone (FIG. 26B). OVA-loaded PS particles targeted with UEA-1 or UEA-1 mimetic did not significantly enhance serum IgG1 antibody titres as compared to OVA-loaded PS particles alone (FIG. 26B).

IgG2a

Immunization with OVA-loaded PS particles did not significantly increase IgG2a titres compared to mice immunized with OVA alone (FIG. 26C). Nor did OVA-loaded PS particles targeted with UEA-1 or UEA-1 mimetic significantly enhance serum IgG2a antibody titres as compared to PS particles alone (FIG. 26C).

It thus appears that immunization with OVA-loaded PS particles induces a significant increase in total IgG and IgG1 antibody titres, but not in total IgG2a antibody titres in serum (as compared to immunization with OVA alone). However, targeting OVA-loaded PS particles with either UEA-1 or UEA-1 mimetic does not appear to induce any further increase in either IgG, IgG1 or IgG2a serum antibody titres when used to immunize mice (as compared to OVA-loaded PS particles alone).

### Example 23

### UEA-1 Targeting of Antigen-Loaded Particles Enhances Antigen-Specific Cytokine Responses in Murine Spleens

In order to determine whether immunisation with antigen-loaded particles targeted with UEA-1 or UEA-1 mimetic enhances antigen-specific cytokine responses in the spleen, the following study was conducted.

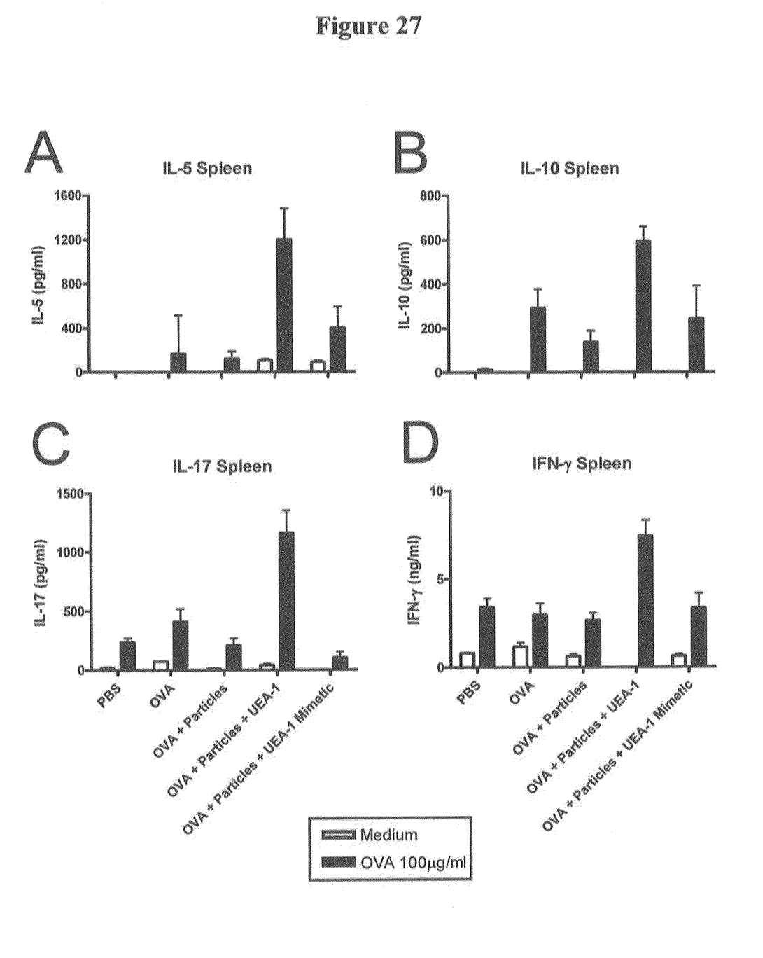

Five groups of BALB/c mice were immunized i.p. (0 days) with OVA alone, OVA-loaded PS particles, OVA-loaded PS particles adsorbed with UEA-1 or OVA-loaded PS particles adsorbed with UEA-1 mimetic, boosted on day 35 with identical vaccines and sacrificed on day 42, at which point their spleens were removed. Antigen-specific IL-5 (FIG. 27A), IL-10 (FIG. 27B) IL-17 (FIG. 27C) and IFN-γ (FIG. 27D) were determined by ELISA on the supernatants from splenocytes (1×10⁶ cells/ml) from the 5 groups of immunized mice stimulated with OVA (100 μg/ml). Results are mean (±SE) responses from five mice per experimental group tested individually in triplicate.

Immunization with OVA-loaded PS particles targeted with UEA-1 induced strong enhancement of antigen-specific IL-5, IL-10, IL-17 and IFN-γ by stimulated splenocytes compared to immunization with OVA alone or with untargeted particles. When the UEA-1 mimetic was used to target OVA-loaded PS particles, splenocytes from these mice did not respond as strongly to OVA stimulation in vitro.

The OVA-specific IL-5, IL-10, IL-17 and IFN-γ cytokine responses in the spleens of mice immunized with OVA-loaded PS particles were increased when UEA-1 was used to target the particles as compared to untargeted particles.

### Example 24

### UEA-1 Targeting of Antigen-Loaded Particles Enhances Antigen-Specific Cytokine Responses by Murine Peritoneal Cells

In order to determine whether immunisation with antigen-loaded particles targeted with UEA-1 or UEA-1 mimetic enhances antigen-specific cytokine responses close to the site of injection, the following analyses were conducted on peritoneal cells.

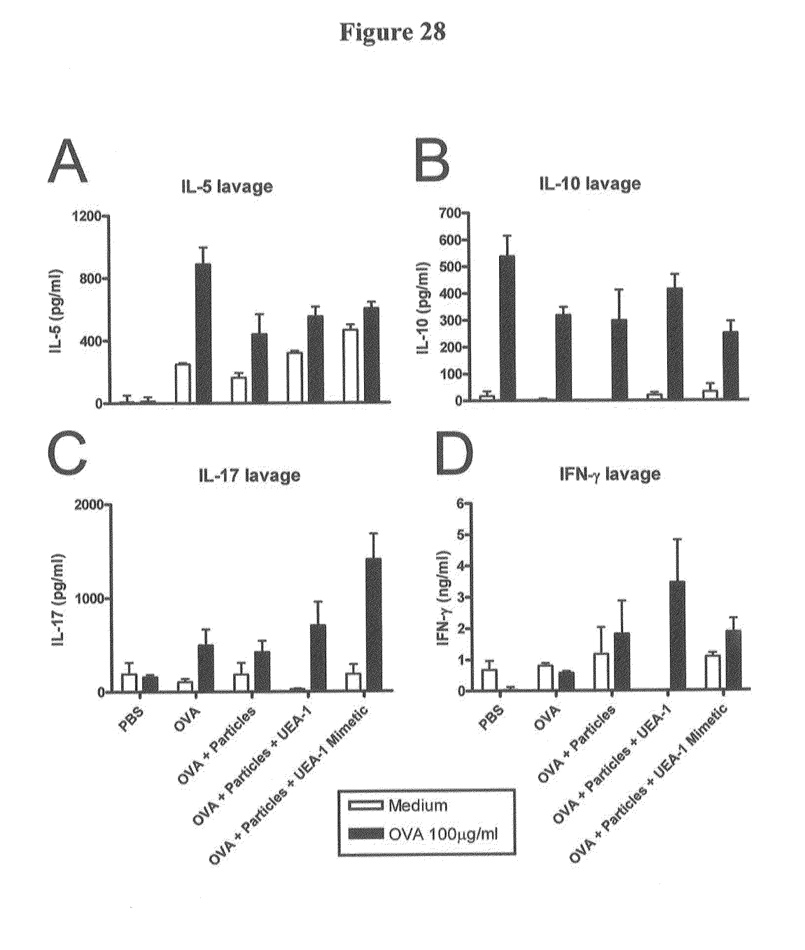

Five groups of BALB/c mice were i.p. immunized once (0 days) with OVA alone, OVA-loaded PS particles, OVA-loaded PS particles adsorbed with UEA-1 or OVA-loaded PS particles adsorbed with UEA-1 mimetic, boosted (day 35) with identical vaccines and sacrificed (day 42), at which point peritoneal cells were obtained by lavage. Antigen-specific IL-5 (FIG. 28A), IL-10 (FIG. 28B), IL-17 (FIG. 28C) and IFN-γ (FIG. 28D) were determined by ELISA on the supernatants from peritoneal cells (1×10⁶ cells/ml) from the 5 groups of immunized mice stimulated with OVA (100 μg/ml). Results are mean (±SE) responses from five mice per experimental group tested individually in triplicate.

Peritoneal cells from mice immunized with OVA alone had stronger antigen-specific IL-5 and IL-17 responses than cells from mice immunized with OVA-loaded PS particles, however cells from mice immunized with the particles alone produced more IFN-γ. Immunization with UEA-1-targeted OVA-loaded PS particles resulted in increased IL-17 and IFN-γ production by peritoneal cells stimulated with OVA compared to mice immunized with particles alone. Antigen-specific IL-17 was strongly produced by peritoneal cells from mice immunized with OVA-loaded PS particles targeted with UEA-1 mimetic. Neither UEA-1 nor UEA-1 mimetic induced an increase in the amount of antigen-specific IL-5 secreted by peritoneal cells from mice immunized with OVA-loaded PS particles. All PMA plus anti-CD3 controls responded with strong cytokine production.

It thus appears that targeting OVA-loaded PS particles with UEA-1 enhances IL-17 and IFN-γ responses in peritoneal cells from immunized mice. Immunisation with particles targeted with UEA-1 mimetic enhances the IL-17 response of peritoneal cells even more so than UEA-1. However, neither UEA-1 nor UEA-1 mimetic targeting of particles appears to enhance the IL-5 response of peritoneal cells of immunized mice.

### Example 25

### The Enhancement of IL-1α and IL-1β Production by Dendritic Cells in Response to UEA-1 Targeted Particles is Dependent on the NLRP3 Inflammasome

In order to determine whether the increase in IL-1α and IL-β production achieved by UEA-1 targeting of PS particles is dependent on the NLRP3 inflammasome, the following experiment was carried out.

BMDCs (6.25×10⁵ cells/ml) from NLRP3^{-/-} and C57BL/6 mice were stimulated with LPS (1 ng/ml) for 6 hours or left unstimulated. After 6 hours, these cells were incubated with PS particles (0.25 mg/ml to 1 mg/ml) or PS particles with UEA-1 mimetic adsorbed to their surface (0.25 mg/ml to 1 mg/ml). After 24 hour incubation, supernatants were assayed for IL-1α and IL-1β by ELISA.

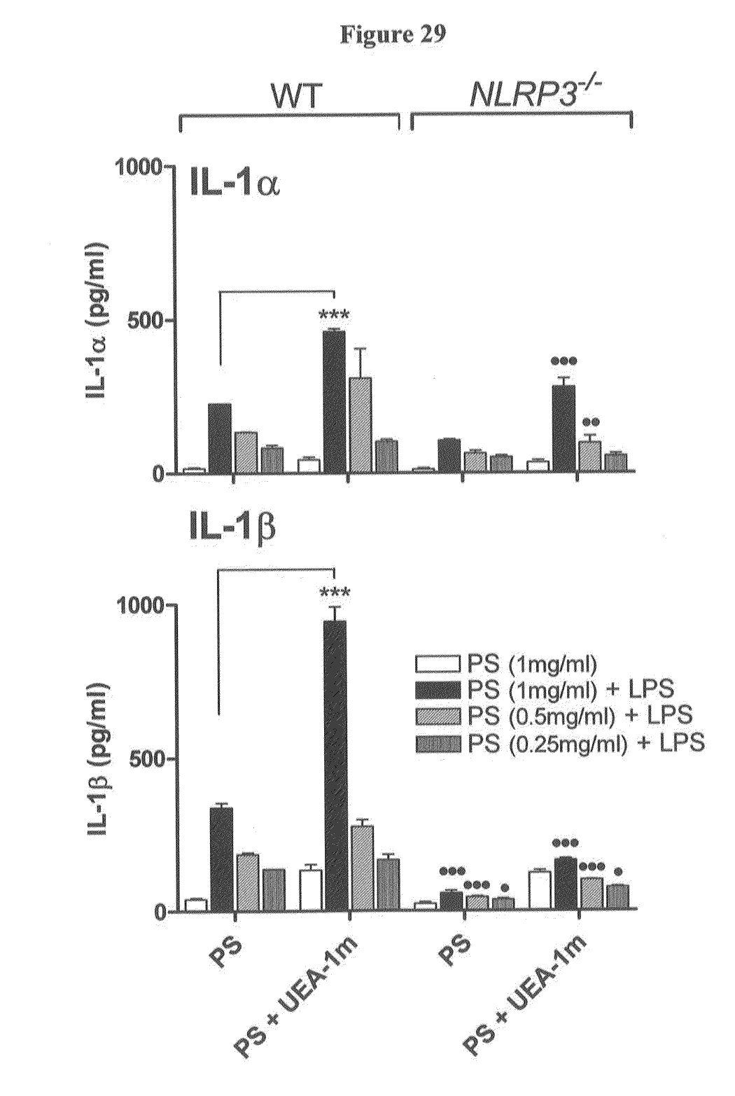

As shown in FIG. 29, PS targeted with UEA-1 mimetic induced higher IL-1α and IL-1β production than untargeted particles in BMDCs isolated from C57BL/6 mice, but the effect is reduced in BMDCs isolated from NLRP3^{-/-} mice.

### Example 26

### UEA-1 Targeting of Nasal Vaccines is Dependent on the NLRP3 Inflammasome

In order to determine whether the effects of UEA-1 targeting may be mediated by the NLRP3 inflammasome, the following study was conducted.

Four groups of C57BL/6 (WT) and NLRP3^{-/-} mice were intranasally immunized three times (0, 14 and 28 days) with PBS, OVA alone, OVA attached to SC-PS particles or OVA attached to SC-PS particles loaded with UEA-1 mimetic and then sacrificed (day 35), at which point cells were isolated from the spleen and the mediastinal lymph nodes.

Cells isolated from the mediastinal lymph nodes of C57BL/6 and NLRP3″ mice in the OVA+UEA-1 mimetic-loaded SC-PS particles group were restimulated on day 35 for 6 hours with control solution (complete RPMI) or OVA (500 μg/ml) in the presence of Brefeldin A (10 μg/ml), which blocked cytokine export from the cell. The cells were fixed and labelled with fluorescent anti-CD3, anti-CD4, anti-CD8, anti-IL-17 and anti-IFNγ antibodies and analyzed with a FACSCantoII™ flow cytometer (BD Biosciences, San Jose, Calif.), and FlowJo™ software (Treestar, Inc., Ashland, Oreg.) were used to analyze. Live CD3⁺CD8⁺ cells were gated upon, and the percentage of IFNγ-positive and IL-17-positive cells within these populations was determined.

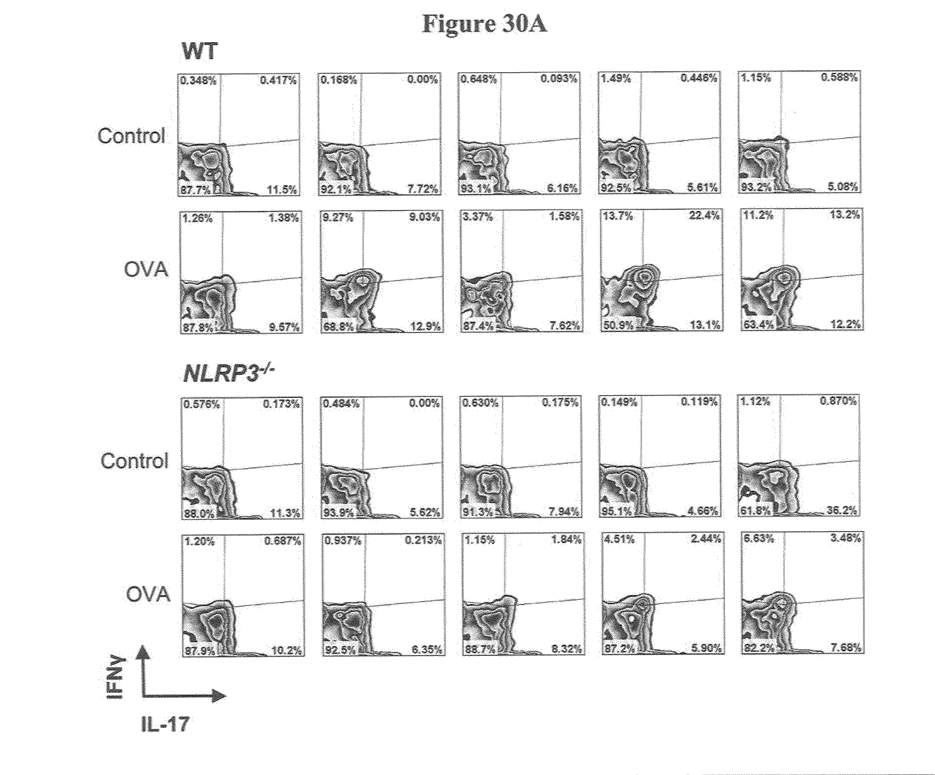

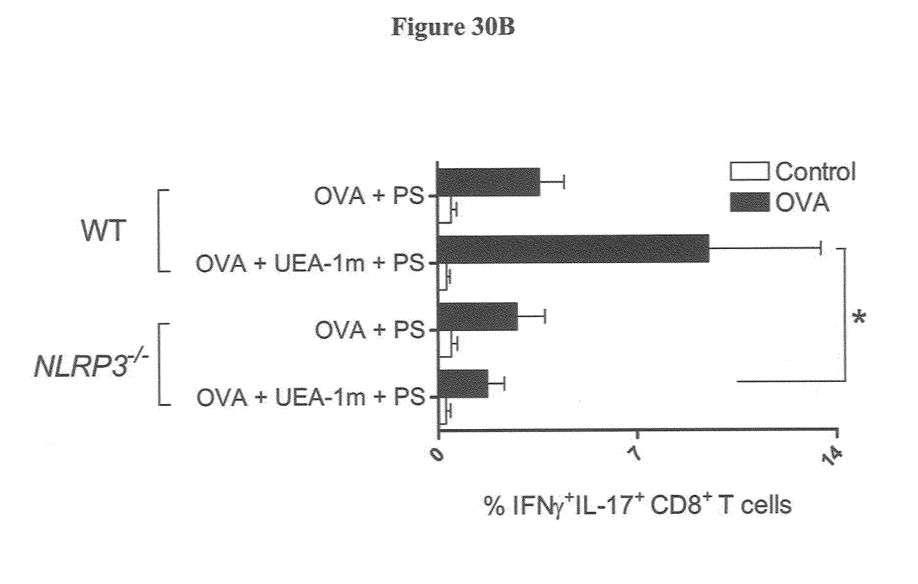

As shown in FIGS. 30A and 30B, intranasally immunizing mice with UEA-1 targeted particles induces an IL-17- and IFNγ-producing population of antigen-specific CD3⁺CD8⁺ T cells in the mediastinal lymph nodes of both C57BL/6 and NLRP3^{-/-} mice, with a greater inducement seen in C57BL/6 mice.

### Example 27

### UEA-1 Targeting of Chitosan Enhances IL-1β Production in Dendritic Cells and is not Dependent on the NLRP3 Inflammasome

In order to determine whether UEA-1 can effectively target chitosan and whether such targeting is dependent on the NLRP3 inflammasome, the following experiment was carried out.

BMDCs (6.25×10⁵ cells/ml) from NLRP3^{-/-} and C57BL/6 mice were stimulated with LPS (1 ng/ml) for 6 hours or left unstimulated. After 6 hours, these cells were incubated with serially diluted chitosan (2 μg/ml) without or without UEA-1 mimetic (50 μg/ml). After 24 hour incubation, supernatants were assayed for IL-1α and IL-1β by ELISA.

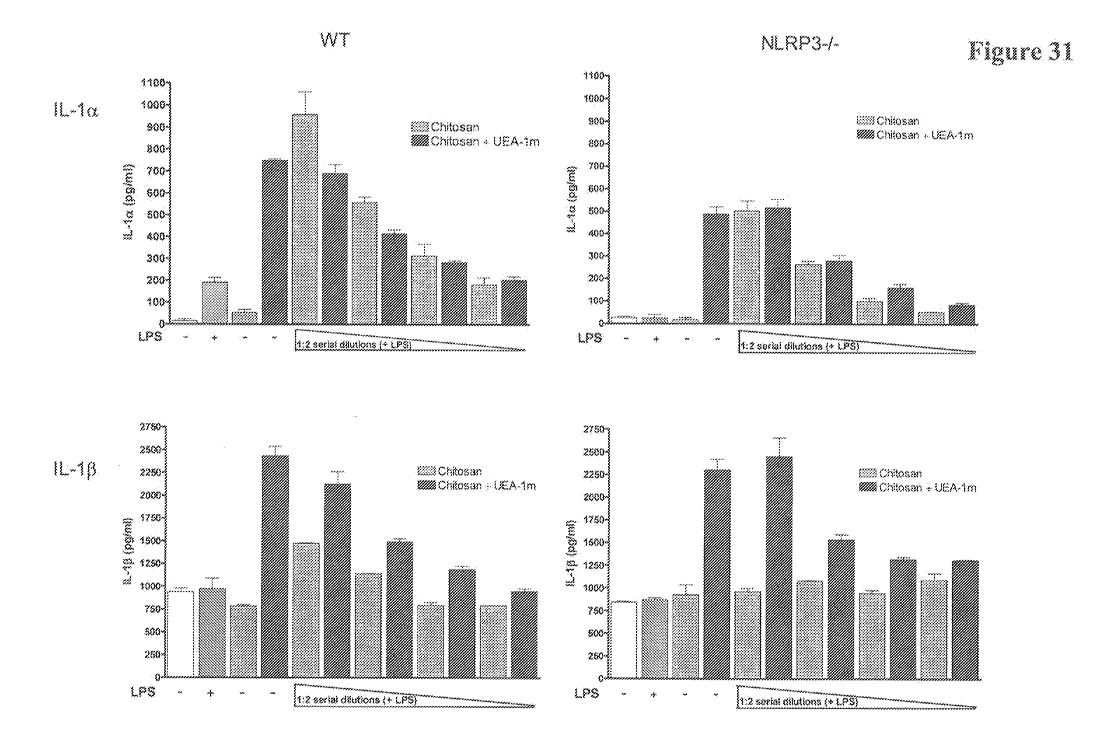

As shown in FIG. 31, UEA-1 targeted chitosan induced higher IL-1β production than untargeted chitosan in BMDCs isolated from C57BL/6 mice, but failed to induce higher IL-1α production. The targeting effect of UEA-1 appeared to be independent of the NLRP3 inflammasome.

### Example 28

### UEA-1 Targeting of Nasal Vaccines is Observed

### Following Intranasal Immunization with a Staphylococcus aureus Antigen

In order to determine whether the effects of UEA-1 targeting is observed following intranasal immunization with a Staphylococcus aureus antigen, the following study was conducted.

Four groups of BALB/c mice were intranasally immunized three times (0, 14 and 28 days) with PBS, ClfA alone, ClfA attached to SC-PS particles or ClfA attached to SC-PS particles loaded with UEA-1 mimetic and then sacrificed (day 35), at which point cells were isolated from the spleen and the mediastinal lymph nodes.

Splenocytes were stimulated with ClfA (0.2 μg/ml) for 72 hours or left unstimulated. After 72 hours, supernatants were assayed for Il-4, IL-10, IL-17 and IFNγ by ELISA.

Cells isolated from the mediastinal lymph nodes were restimulated on day 35 for 6 hours with control solution (complete RPMI) or ClfA (10 μg/ml) in the presence of Brefeldin A (10 μg/ml), which blocked cytokine export from the cell. The cells were fixed and labelled with fluorescent anti-CD3, anti-CD4, anti-CD8, anti-IL-17 and anti-IFNγ antibodies and analyzed with a FACSCantoII™ flow cytometer (BD Biosciences, San Jose, Calif.), and FlowJo™ software (Treestar, Inc., Ashland, Oreg.) were used to analyze. Live CD3⁺CD4⁺ or CD3⁺CD8⁺ cells were gated upon, and the percentage of IFNγ-positive and IL-17-positive cells within these populations was determined (FIG. 30a).

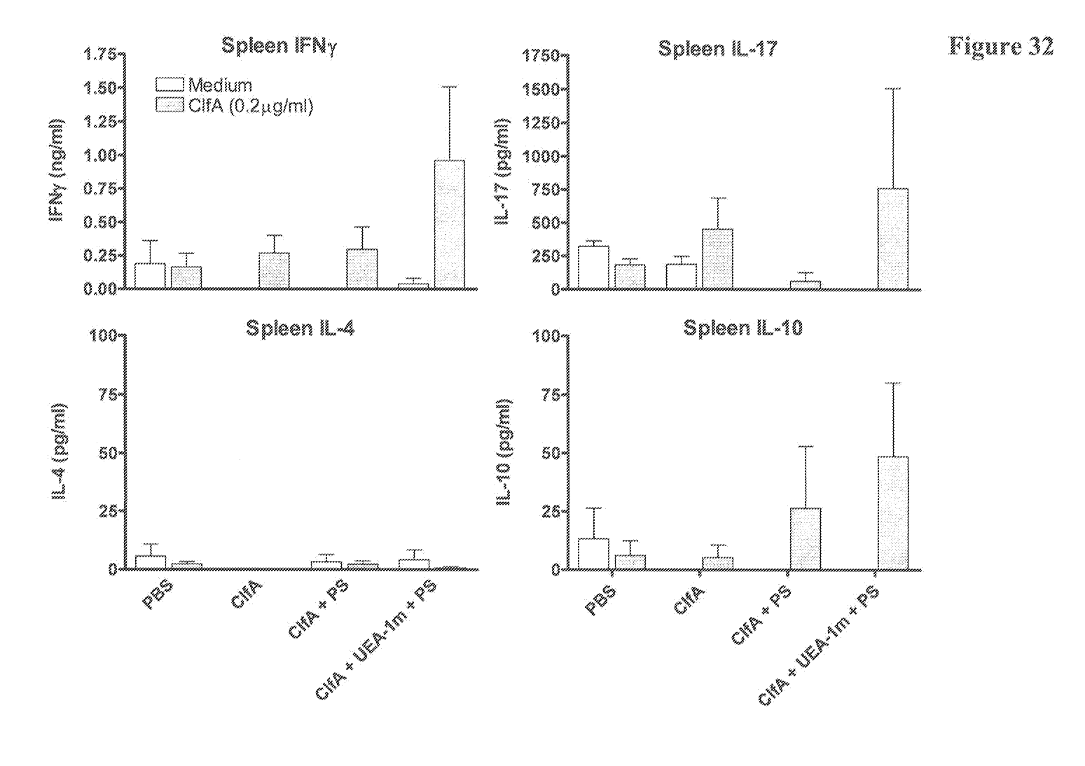

As shown in FIG. 32, intranasally immunizing mice with UEA-1 targeted particles coated with ClfA, a fibrinogen-binding surface protein of Staphylococcus aureus (Foster and Hook, TRENDS MICROBIOL. 6:484 (1998); Narita et al., INFECT. IMMUN. 78:4234 (2010)) increases the ex vivo production of IL-17- and IFNγ by splenocytes.

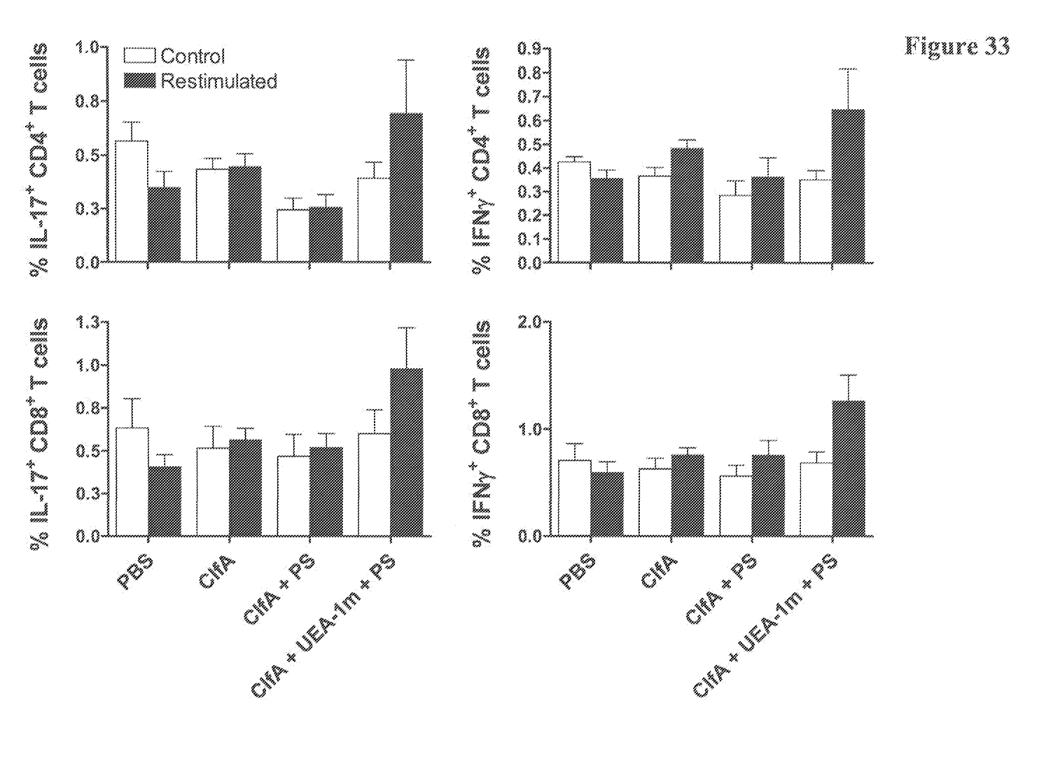

As shown in FIG. 33, intranasally immunizing mice with UEA-1 targeted particles coated with ClfA also induces IL-17- and IFNγ-producing populations of antigen-specific CD3⁺CD4⁺ and CD3⁺CD8⁺ T cells in the mediastinal lymph nodes of both BALB/c mice.

### Example 29

### Immune Response May be Selectively Activated by Targeting Antigens with Different Plant Lectins

In order to determine whether cellular and/or humoral immune responses can be selectively activated by targeting antigens with different plant lectins, the following study was conducted.

Seven groups of BALB/c mice were i.p. immunized once (0 days) with PBS, ClfA alone, ClfA attached to SC-PS particles, ClfA attached to SC-PS particles loaded with UEA-1, ClfA attached to SC-PS particles loaded with UEA-1 mimetic, ClfA attached to SC-PS particles loaded with PHA-L or ClfA attached to SC-PS particles loaded with SBA and then sacrificed (day 14), at which point cells were isolated from the spleen and the peritoneal cavity.

Splenocytes were stimulated with ClfA (10 μg/ml) for 72 hours or left unstimulated. Peritoneal exudate cells were stimulated with anti-CD3 (0.5 μg/ml) (BD Pharmingen, San Diego, Calif.) and PMA (25 ng/ml) (Sigma-Aldrich, St. Louis, Mo.) for 72 hours or left unstimulated. After 72 hours, supernatants were assayed for Il-4, IL-10, IL-17 and IFNγ by ELISA.

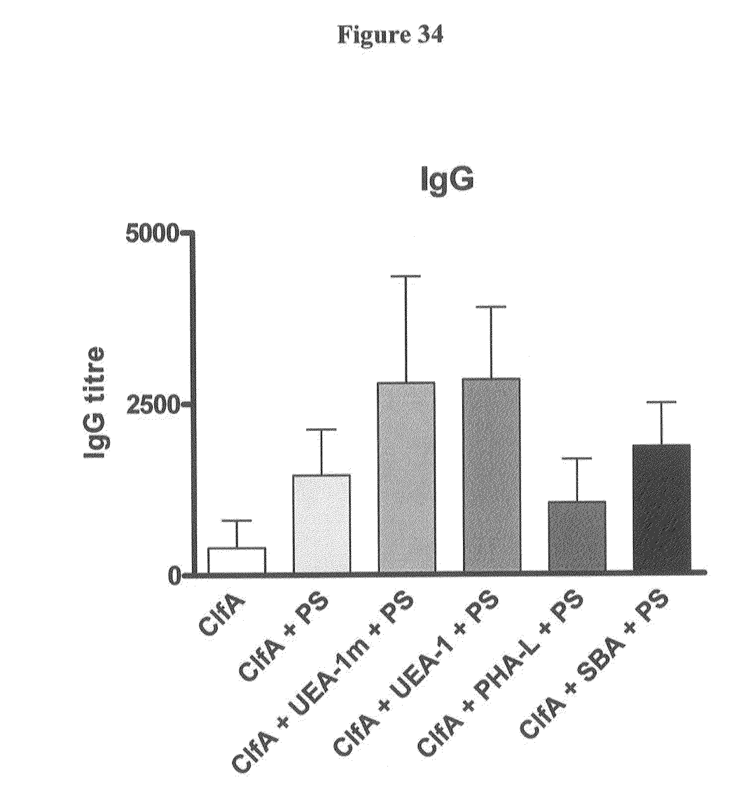

As shown in FIG. 34, attaching ClfA to SC-PS particles increased the production of antigen-specific antibodies, compared to ClfA alone. The production of antigen-specific antibodies was increased by co-attachment of UEA-1, UEA-1 mimetic or SBA to the SC-PS particles.

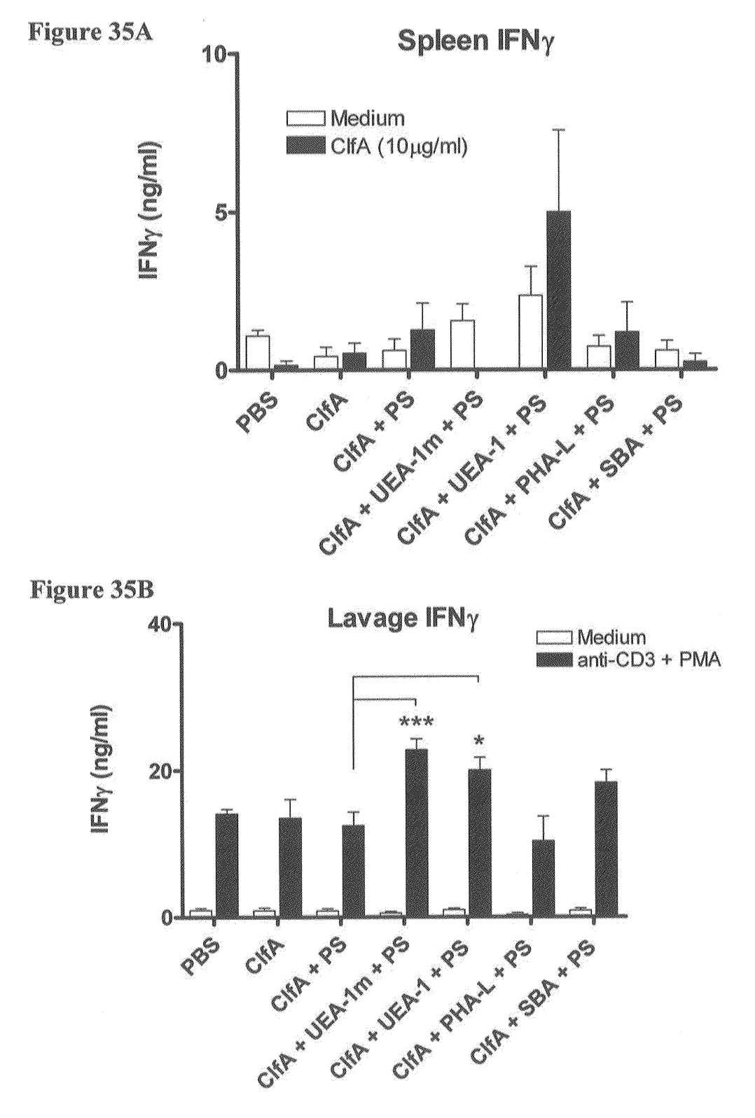

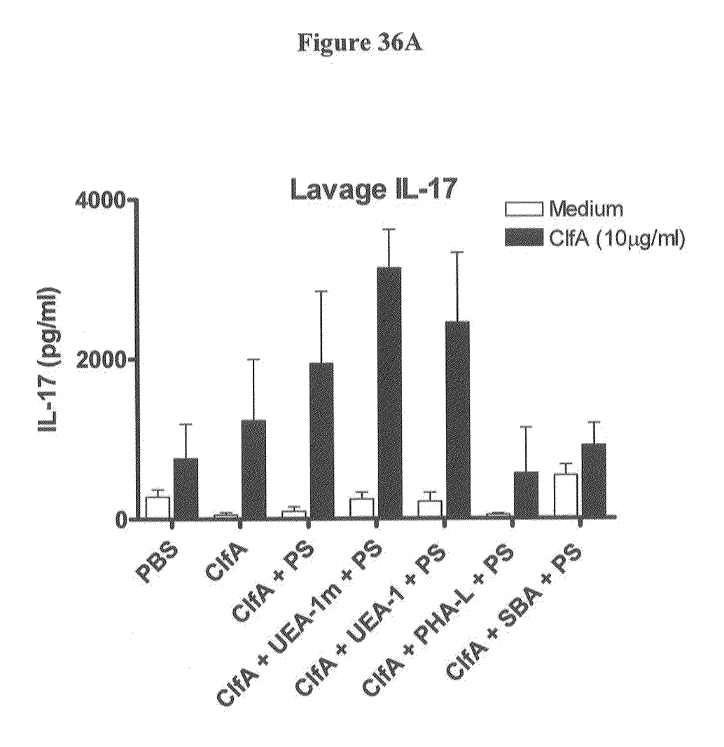

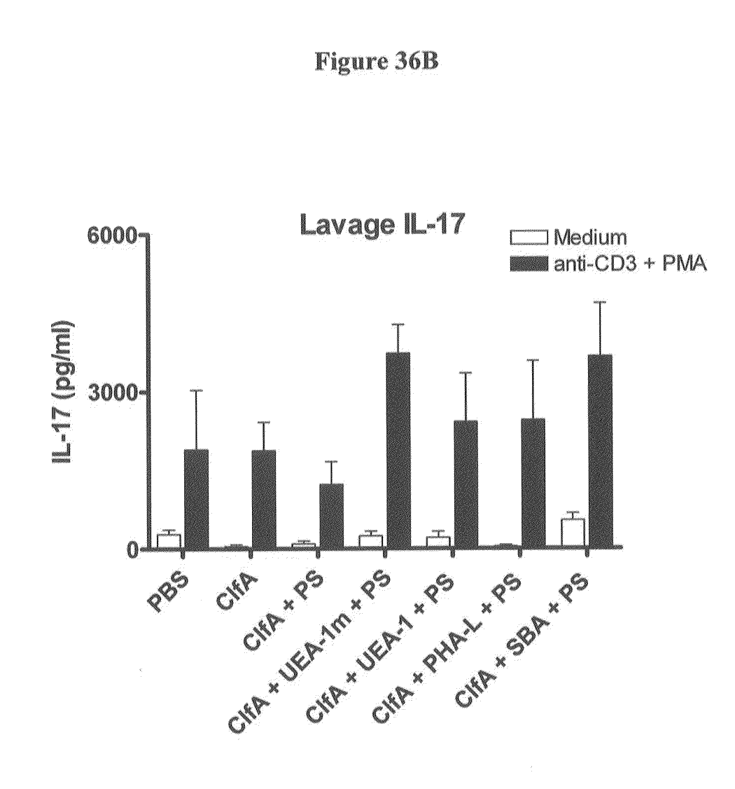

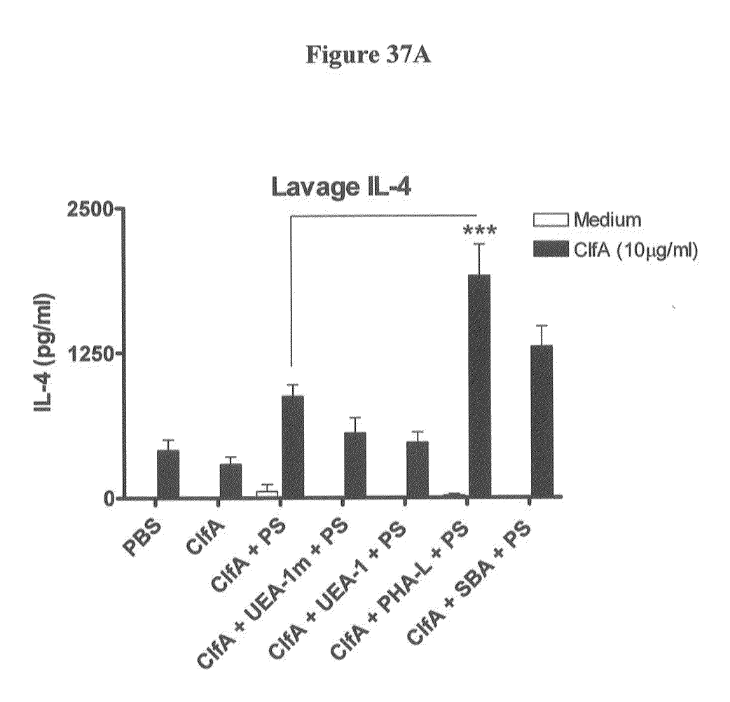

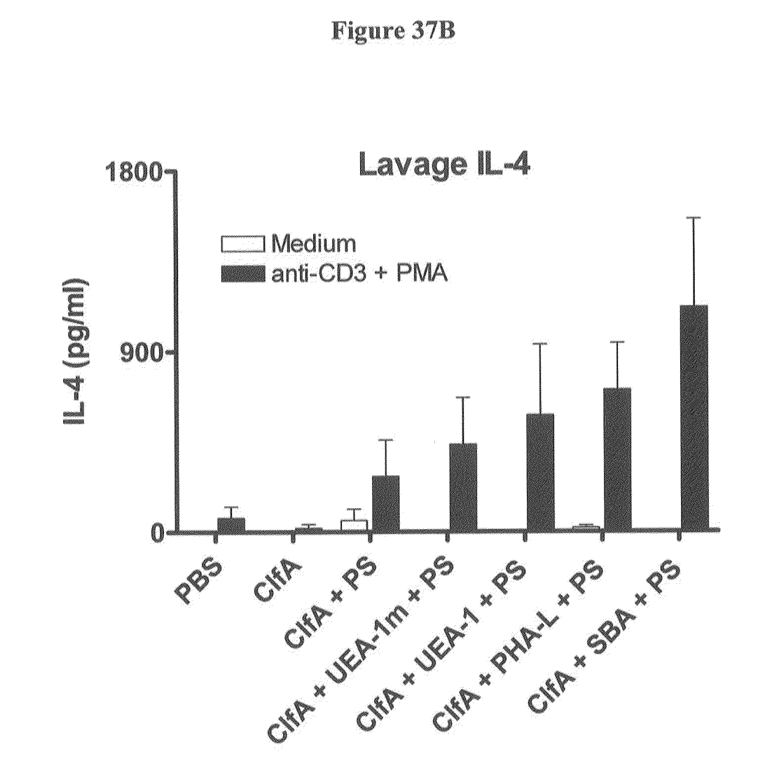

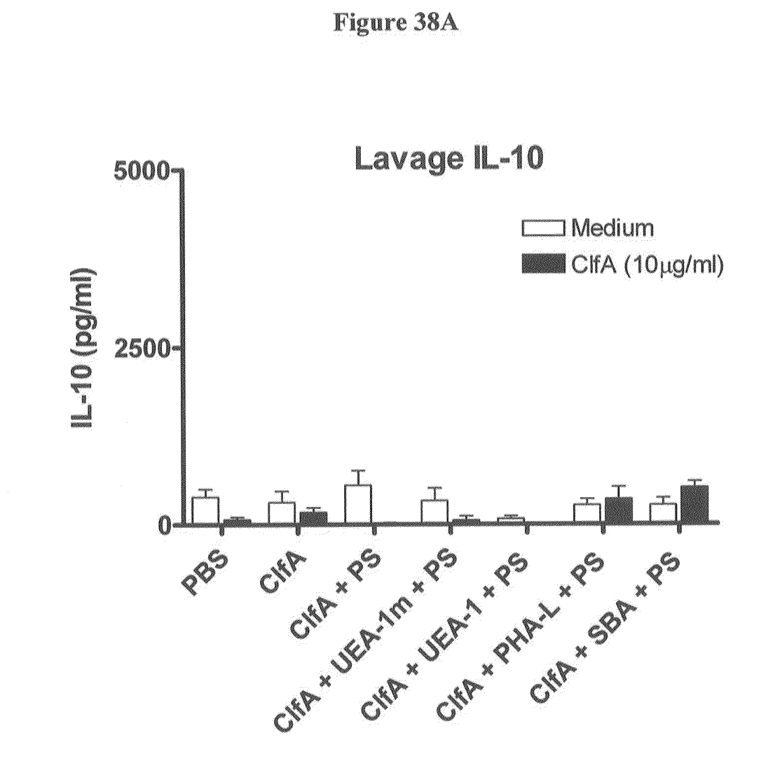

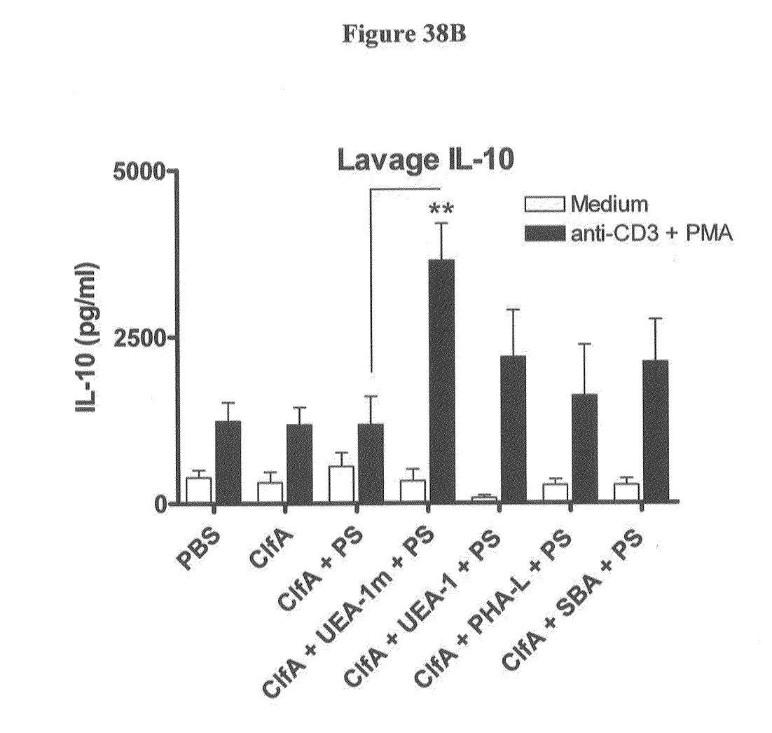

As shown in FIGS. 35A-38B, mice immunized with ClfA attached to SC-PS particles loaded with UEA-1 or UEA-1 mimetic displayed increased IFNγ and IL-17 production in cells isolated from the spleen and peritoneal cavity (FIGS. 35A-36B), whereas mice immunized with ClfA attached to SC-PS particles loaded with PHA-L or SBA displayed increased Il-4 and IL-10 production in cells isolated from the spleen and peritoneal cavity (FIGS. 37A-38B).

### DISCUSSION

M cells have been shown to take up orally administered microparticles and are thus considered a target for vaccination with antigen-loaded microparticles (which gives rise to a primarily humoral immune response). One obstacle to oral vaccination with microparticles is that the microparticles may pass through the digestive tract without coming into contact with M cells (by being excreted or becoming trapped, for example). One study also estimated that only 10% of microparticles would be taken up by M cells. To overcome this, microparticles have been targeted with lectins that can bind to glycoproteins of the M cell's surface. UEA-1 is a lectin from the gorse plant that, when attached to microparticles, was shown to target murine M cells and increase particle uptake. UEA-1 targeting to M cells has also been shown to increase oral vaccine efficacy in mice.

We have herein shown that targeting particles to leukocytes with plant lectins, such as Con A, DBA, DSL, GSL I, GSL II, Jac, LEL, PHA-L, PHA-E, PNA, SBA, UEA-1, VVL, and WGA, or mimetics thereof, leads to increased particle uptake and increased immune response. In particular, we have shown that targeting particles with lectins can dramatically increase both the number of cells taking up the particles and the number of particles taken up per leukocyte. Notably, our results demonstrate that the particles were taken into the cytoplasm, as opposed to merely sticking to the membrane, indicating that lectin-mediated targeting may act via α-L-fucose, leading to a receptor-mediated increase in particle uptake. Moreover, our results demonstrate that plant lectins and mimetics thereof can be used to target leukocytes following non-oral routes of administration (e.g., intraperitoneal administration and/or nasal administration).

The ability to increase uptake by dendritic cells may have beneficial applications in vaccination and immunotherapy. Dendritic cells have been recognised as valid targets for generating cellular immune responses against various antigens, including intra-cellular pathogens (such as HIV, malaria and TB), cancer and allergens. Lectin-mediated targeting thus presents an opportunity to modulate dendritic cells to elicit the desired response. As our results demonstrate, a variety of plant lectins may be used to target particles to dendritic cells. The increase in particle uptake per dendritic cell when targeted with UEA-1 was much greater after a two-hour incubation in vitro, as compared to a shorter one-hour incubation period. There appears to be no limit to the amount of particles that dendritic cells will take up, even to the point at which cell lysis occurs.

Compositions and methods of the present invention may also be used to elicit immune responses by targeting other leukocyte types. Our results demonstrate that lectin-mediated targeting also induces dramatic increases in the number of various splenocyte populations taking up particles and also increases the number of particles taken up per cell. For example, the cellular uptake of particles into splenic monocytes was greatly increased when the particles were conjugated to UEA-1 (FIG. 9A and FIG. 10). Monocytes have been shown to be among the first leukocyte populations to migrate to the site of injection of alum and MF59. Studies on the clinical adjuvants alum and MF59 have shown that both adjuvants can induce monocyte differentiation into dendritic cells, which were shown to be extremely potent APCs and T cell activators. Thus, targeting monocytes with antigen-loaded particles could induce the differentiation of active, antigen-presenting cells.

Our results also demonstrate that particles targeted with either plant lectins and mimetics thereof significantly enhance IL-1α and IL-β production by dendritic cells, as compared to untargeted particles. Moreover, we have confirmed that the IL-1β produced in response to lectin-targeted particles and lectin mimetic-targeted particles is active IL-1β. Although targeting with UEA-1 increased IL-1α and IL-1β production to a greater degree than did targeting with UEA-1 mimetic, these results indicate that both wild type lectins and lectin mimetics may be viable candidates for targeting antigens in vivo.

A comparison of several methods for attaching plant lectins to particles shows that more efficient enhancement of IL-1α and IL-1β may be achieved when biotinylated lectins are conjugated to SC-PS particles, as opposed to adsorbing the lectins to PS particles.

Particulate adjuvants such as alum are well established clinical adjuvants. Most vaccines rely on the induction of a humoral immune response, which is sustained by memory B cells. However, many diseases for which no vaccines are available require a cellular and not a humoral response for protection. HIV, malaria, tuberculosis and cancer are all malignancies that reside within cells. As these are intracellular, they are more difficult to detect than extracellular threats. These have also evolved mechanisms to evade immune detection, further complicating the mounting of an effective immune response. This has made developing vaccines against these very difficult. Central to the clearance of these threats is the cellular immune response. Targeting vaccines to dendritic cells and inducing a strong CD4⁺ T_{H}1 cell mediated response is key to resolving and mounting efficient protection from these threats.

Having shown that attachment of lectins to particles increases particle uptake in dendritic cells and that this induces increased cytokine production in vitro, we proceeded with an in vivo study to compare the ability of both UEA-1 and UEA-1 mimetic targeting to enhance antigen-specific responses elicited by intraperitoneal injection of antigen-laden particles.

Targeting of OVA-loaded particles with UEA-1 or UEA-1 mimetic did not induce any enhancement of antigen-specific IgG, IgG1 or IgG2a serum antibody titres in i.p. immunized mice.

Enhancement of IFN-γ production by peritoneal cells from mice immunized with a UEA-1-targeted formulation suggest that a T_{H}1 response is primed close to the site of injection in vivo. Splenocyte T_{H}17 responses are also increased following immunization with UEA-1 targeted particles compared to particles alone. Very high levels of antigen-specific IL-17 were produced by peritoneal cells from mice immunized with mimetic targeted formulations, indicating a T_{H}17 response in vivo. This suggests that targeting with UEA-1 or UEA-1 mimetic induces a much more effective cellular immune response to antigen than untargeted particles loaded with antigen. A T_{H}1 and T_{H}17 type response is required for the clearance of malaria and tuberculosis. Lectin-mediated targeting of particles containing antigens from these pathogens could provide a possible vaccination strategy against these diseases.

Splenocytes from mice immunized with PS particles loaded with antigen and targeted with UEA-1 elicited strong IL-5, IL-10, IL-17 and IFN-γ responses when stimulated with antigen in vitro.

Targeting antigen-loaded particles with plant lectins and mimetics thereof induces an enhancement of cellular responses in vivo. This could provide a means for vaccinations where dendritic cells control the fate of the immune response. Establishment of tolerance by immunotherapy relies on dendritic cells to induce regulatory T cells so as to induce tolerance to the allergen. Dendritic cell priming ex vivo has shown promise as a method for exposing dendritic cells to cancer antigens before being re-injected into the host to mount a cytotoxic T cell response against the threat. However it would be much more advantageous if vaccine delivery systems could target known cancer antigens to dendritic cells in vivo, thus priming the immune response from within. Dendritic cell activation is paramount for the induction of the correct T cell response, making them important targets for the development of new vaccines and new vaccination strategies such as sublingual vaccination seems to represent a new novel site of vaccine delivery.

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications are incorporated in there entireties into this application by reference herein in order to more fully describe the state of the art to which this invention pertains.

1-3. (canceled)

4. A method of targeting an antigen to leukocytes in a subject, comprising administering to the subject a conjugate comprising the antigen and a plant lectin or a mimetic thereof.

5-7. (canceled)

8. A method of enhancing an immune response to an antigen in a subject, comprising administering to the subject a conjugate comprising the antigen and a plant lectin or a mimetic thereof.

9. The method of claim 8, wherein the immune response comprises a cellular immune response.

10. (canceled)

11. The method of claim 4, wherein the method results in an increase in the number of leukocytes taking up the antigen and/or the amount of antigen taken up per leukocyte relative to a method comprising the administration of a composition that lacks a plant lectin or a mimetic thereof.

12-13. (canceled)

14. The method of claim 4, wherein the plant lectin or mimetic thereof is PHA-L, a PHA-L mimetic, PHA-E, a PHA-E mimetic, DBA, a DBA mimetic, Con A, a Con A mimetic, WGA, a WGA mimetic, PNA, a PNA mimetic, UEA-1, a UEA-1 mimetic, PSA, a PSA mimetic, LEL, an LEL mimetic, VVL, a VVL mimetic, Jac, a Jac mimetic, GSL II, a GSL II mimetic, GSL I, a GSL I mimetic, SBA, an SBA mimetic, DSL or a DSL mimetic.

15-20. (canceled)

21. The method of claim 4, wherein the leukocytes are selected from the group consisting of dendritic cells, monocytes, and granulocytes.

22. (canceled)

23. The method of claim 4, wherein the conjugate is administered to the subject by intraperitoneal injection or intranasal administration.

24. (canceled)

25. The method of claim 4, wherein the conjugate is administered in a composition comprising a particle.

26-31. (canceled)

32. The method of claim 25, wherein the particle comprises polystyrene, poly(lactic acid), poly(glycolic acid) or poly(lactic-co-glycolic acid).

33. (canceled)

34. The method of claim 25, wherein the antigen is embedded in or attached to the surface of the particle.

35-36. (canceled)

37. The method of claim 25, wherein the plant lectin or mimetic thereof is embedded in or attached to the surface of the particle.

38-39. (canceled)

40. The method of claim 25, wherein the particle is coated with streptavidin, and wherein one or both of the antigen and the plant lectin or mimetic thereof is biotinylated and attached to the surface of the particle via an interaction between a biotin attached thereto and the streptavidin coating on the particle.

41. A composition for intraperitoneal delivery of an antigen to leukocytes, comprising:
an antigen;
a plant lectin or a mimetic thereof; and
a pharmaceutically acceptable carrier,
wherein the antigen and the plant lectin or mimetic thereof form a conjugate.

42. (canceled)

43. The composition of claim 41, wherein the plant lectin or mimetic thereof is PHA-L, a PHA-L mimetic, PHA-E, a PHA-E mimetic, DBA, a DBA mimetic, Con A, a Con A mimetic, WGA, a WGA mimetic, PNA, a PNA mimetic, UEA-1, a UEA-1 mimetic, PSA, a PSA mimetic, LEL, an LEL mimetic, VVL, a VVL mimetic, Jac, a Jac mimetic, GSL H, a GSL H mimetic, GSL I, a GSL I mimetic, SBA, an SBA mimetic, DSL or a DSL mimetic.

44-51. (canceled)

52. The composition of claim 41, wherein the plant lectin or mimetic thereof targets dendritic cells, monocytes or granulocytes.

53. (canceled)

54. The composition of claim 41, wherein the composition further comprises a particle.

55-60. (canceled)

61. The composition of claim 54, wherein the particle comprises polystyrene, poly(glycolic acid), poly(lactic acid), or poly(lactic-co-glycolic acid).

62. (canceled)

63. The composition of claim 54, wherein the antigen is embedded in or attached to the surface of the particle.

64-65. (canceled)

66. The composition of claim 54, wherein the plant lectin or mimetic thereof is embedded in or attached to the surface of the particle.

67-68. (canceled)

69. The composition of claim 54, wherein the particle is coated with streptavidin, and wherein one or both of the antigen and the plant lectin or mimetic thereof is biotinylated and attached to the surface of the particle via an interaction between a biotin attached thereto and the streptavidin coating on the particle.
